(19) United States
(12) Patent Application Publication
(10) Pub. No.: US 2024/0218424 A1
(43) Pub. Date: Jul. 4, 2024

(54) METHODS FOR TETHERING RIBONUCLEIC ACIDS IN BIOLOGICAL SAMPLES

(71) Applicant: 10x Genomics, Inc., Pleasanton, CA (US)

(72) Inventors: Justin COSTA, Union City, CA (US); Meiliana TJANDRA, Dublin, CA (US)

(21) Appl. No.: 18/391,284

(22) Filed: Dec. 20, 2023

Related U.S. Application Data
(60) Provisional application No. 63/461,871, filed on Apr. 25, 2023.
(60) Provisional application No. 63/434,330, filed on Dec. 21, 2022.

(51) Int. Cl. C12Q 1/6806; C12Q 1/37; C12Q 1/48; C12Q 1/6844

(52) U.S. Cl. CPC C12Q 1/6806; C12Q 1/37; C12Q 1/485; C12Q 1/6844

(57) ABSTRACT

The present disclosure relates in some aspects to methods and compositions for immobilizing ribonucleic acid analytes in biological samples, and more specifically fragmented ribonucleic acids. Ribonucleic acid analytes may be tethered covalently or non-covalently to exogenous or endogenous molecules in a biological sample, for example, cross-linked directly to a polymerized three-dimensional matrix.

### CROSS-REFERENCE TO RELATED APPLICATIONS

This application claims priority to U.S. Provisional Patent Application No. 63/434,330 filed Dec. 21, 2022, and U.S. Provisional Patent Application No. 63/461,871 filed Apr. 25, 2023, entitled “METHODS FOR TETHERING RIBONUCLEIC ACIDS IN BIOLOGICAL SAMPLES,” which is incorporated by reference in its entirety for all purposes.

### FIELD

The present disclosure relates in some aspects to methods for analyzing biological samples, and more specifically, methods for analyzing fragmented RNA.

### BACKGROUND

Despite improvements in transcriptomic analysis, many nucleic acid analytes present in biological samples can be lost throughout sample preparation using standard protocols and reagents, e.g., permeabilization and de-crosslinking, to enable analysis and imaging, such as by fluorescence in situ hybridization. Although the extent of losses of these uncaptured nucleic acid analytes out of the RNA transcripts for a given sample remain unknown, the un-sequenced RNA analytes nonetheless represent substantial segment of the overall transcriptome that remains omitted by analysis and constitute a significant gap in our knowledge for characterizing disease states of tissue samples.

Existing treatments to capture such analytes typically rely upon hybridization of the target RNA with complementary nucleic acid probes and cross-linking to the probes. However, these methods may still suffer from loss of RNA analytes in sample preparation. In instances where limited sample treatment is performed in order to preserve RNA, such target analytes are preserved intact but may be blocked by proteins, ribosomes, etc., that are also present, and, thus, may require large quantities of probe materials to obtain signal as a result.

Thus, improved methods and techniques for sequencing ribonucleic acids, particularly fragmented ribonucleic acids, are needed. Provided herein are methods and compositions that address such and other needs.

### SUMMARY

The methods and kits of the present disclosure provide means to anchor (or immobilize) ribonucleic acids which otherwise might be removed or destroyed during sample preparation, particularly fragmented ribonucleic acids, to exogenous or endogenous molecules present in a biological sample prior to sample work-up, thereby enabling downstream analysis of the ribonucleic acid analytes.

In one aspect, provided herein is a method, comprising: (a) contacting a biological sample comprising a ribonucleic acid with a formylation reagent, wherein the ribonucleic acid comprises a 2′,3′-vicinal diol and the formylation reagent converts the 2′,3′-vicinal diol moiety into a 2′3′-dialdehyde moiety; and (b) contacting the biological sample with an attachment agent comprising at least one aldehyde-reactive group capable of reacting with at least one aldehyde of the 2′,3′-dialdehyde moiety of the ribonucleic acid to form a covalent bond and an attachment moiety capable of attaching covalently or non-covalently to an exogenous or endogenous molecule in the biological sample, thereby immobilizing the ribonucleic acid in the biological sample.

In any of the embodiments herein, the ribonucleic acid can be a fragmented ribonucleic acid. In any of the embodiments herein, the 2′,3′-vicinal diol can be a fragmented 3′ end of the ribonucleic acid. In any of the embodiments herein, the 2′,3′-vicinal diol can be generated from a fragmented ribonucleic acid having a 2′,3′cyclo-phosphate fragmentation at the 3′-terminal end or a 2′ hydroxyl and a 3′ phosphate fragmentation at the 3′-terminal end. In any of the embodiments herein, the 2′,3′-vicinal diol can be provided by contacting the fragmented ribonucleic acid with a 3′ phosphatase. For instance, in some embodiments the methods provided herein comprise contacting the fragmented ribonucleic acid with a 3′ phosphatase to provide the 2′,3′-vicinal diol. In any of the embodiments herein, the 3′ phosphatase can be T4 polynucleotide kinase. In any of the embodiments herein, the biological sample can be treated with degradation agent to induce fragmentation of ribonucleic acids. In some embodiments, the degradation agent can be an RNase or restriction enzyme. In any of the embodiments herein, the ribonucleic acid can comprise a 5′ cap and the 2′,3′-vicinal diol can be in the 5′ cap. In any of the embodiments herein, the 5′ cap can be a 7-methylguanosine cap.

In any of the embodiments herein, the formylation reagent can be an oxidant. In some embodiments, the oxidant can be sodium (meta)periodate. In any of the embodiments herein, the attachment moiety can be capable of reacting with the exogenous or endogenous molecule in the biological sample to form a covalent bond. In any of the embodiments herein, when attachment agent comprises one aldehyde-reactive group and one attachment moiety, and the attachment moiety can comprise a second reactive group capable of reacting with the exogenous or endogenous molecule in the biological sample to form a covalent bond.

In any of the embodiments herein, the attachment moiety can be or comprise an alkenyl, allyl or vinyl moiety, an amide moiety, an alcohol moiety, a polyol moiety, a furan moiety, a maleimide moiety, a norbornene moiety, a thiol moiety, a phenol moiety, a urethane moiety, a cyano moiety, an isocyanate moiety, an isothiocyanate moiety, an ether moiety, a dextran moiety, or an alginate moiety. In any of the embodiments herein, the attachment moiety can be or comprise a click functional group. In any of the embodiments herein, the step of contacting the biological sample and attachment agent further comprises contacting the biological sample with one or more reagents or under suitable conditions to facilitate the formation of a covalent bond between the attachment moiety of the attachment agent and the exogenous or endogenous molecule in the biological sample. In any of the embodiments herein, the attachment moiety can be capable of attaching non-covalently to the exogenous or endogenous molecule in the biological sample. In any of the embodiments herein, the attachment agent can be biotinylated.

In any of the embodiments herein, the attachment agent can be a compound of formula (I)

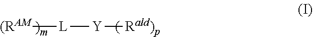

or a salt thereof, wherein each R^{ald} is independently an aldehyde-reactive group; Y is —CH₂CH₂— or —O—; L is a bond or a linker moiety; each R^{AM} is independently an attachment moiety; m is an integer from 1 to 4; and p is an integer from 1 to 4. In any of the embodiments herein, each R^{ald} can be independently an amine moiety, an amide moiety, an alcohol moiety, a thiol moiety, a cyano moiety, an ylide moiety, a hydrazide, a hydroxylamine, a hydrazine, a thiosemicarbazone, a hydrazine carboxylate, or an arylhydrazide, or any combination thereof. In any of the embodiments herein, each R^{AM} can be independently an acrylate moiety, methacrylate moiety, acrylamide moiety, methacrylamide moiety, biotinyl moiety, dextrin moiety, a click moiety, a thiol moiety, norbornenyl moiety, furanyl moiety, alkyl ester moiety, or maleimidyl moiety. In any of the embodiments herein, the attachment agent can be multifunctional and can comprise at least two aldehyde-reactive groups or at least two attachment moieties. In any of the embodiments herein, the attachment agent can comprise at least two aldehyde-reactive groups. In any of the embodiments herein, the attachment agent can comprise at least two attachment moieties.

In any of the embodiments herein, the attachment agent can be bifunctional, comprising one aldehyde-reactive group and one attachment moiety. In any of the embodiments herein, the attachment agent can be a compound of formula (I-a)

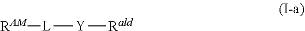

or a salt thereof, wherein R^{ald} is an aldehyde-reactive group; Y is —CH₂CH₂— or —O—; L is a bond or a linker moiety; and R^{AM} is independently an attachment moiety. In any of the embodiments herein, L can be the group

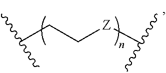

wherein Z is CH₂, O, S, or NH; and n is an integer from 0 to 50.

In any of the embodiments herein, the compound of formula (I-a) can be a compound of formula (III-a)

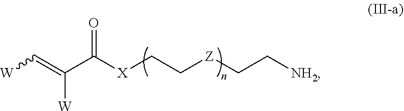

or a salt thereof, wherein each W is independently H or CH₃; X is NH or O; Z is CH₂, O, S, or NH; and n is an integer from 0 to 50. In any of the embodiments herein, the attachment agent can comprise N-(2-aminoethyl)methacrylamide, 2-aminoethyl methacrylate, or 2-aminoethyl (E)-but-2-enoate. In any one or more of the embodiments herein, the attachment agent can be N-(2-aminoethyl)methacrylamide. In any one or more of the embodiments herein, the attachment agent can be 2-aminoethyl methacrylate. In any one or more of the embodiments herein, the attachment agent can be 2-aminoethyl (E)-but-2-enoate.

In any of the embodiments herein, the compound of formula (I-a) can be a compound of formula (III-b)

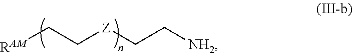

or a salt thereof, wherein R^{AM} is a biotinyl moiety, dextrin moiety, a click moiety, a thiol moiety, norbornenyl moiety, furanyl moiety, alkyl ester moiety, or maleimidyl moiety; Z is CH₂, O, S, or NH; and n is an integer from 0 to 50.

In any of the embodiments herein, the step of contacting the biological sample and attachment agent may further comprise contacting the biological sample with a reducing agent. In some embodiments, the reducing agent can be sodium borohydride. In any of the embodiments herein, the exogenous molecule can be a matrix-forming agent and the method can further comprise: (c) contacting the biological sample with a matrix-forming agent; and (d) forming a three-dimensional polymerized matrix from the matrix-forming agent, thereby embedding the biological sample in the three-dimensional polymerized matrix and anchoring the ribonucleic acid to the three-dimensional polymerized matrix.

In any of the embodiments herein, the method can further comprise clearing the biological sample embedded in the three-dimensional polymerized matrix. In any of the embodiments herein, the biological sample can be cleared with a detergent, a lipase, and/or a protease. In any of the embodiments herein, the biological sample is cleared with a detergent and a protease. In any of the embodiments herein, the detergent can comprise a non-ionic surfactant or anionic surfactant. In any one or more of the embodiments herein, the detergent can comprise SDS, tergitol, NP-40, saponin, polyethylene glycol p-(1,1,3,3-tetramethylbutyl)-phenyl ether, or polysorbate 20, or any combinations thereof. In any of the embodiments herein, the protease can comprise proteinase K, pepsin, or collagenase, trypsin, dispase, thermolysin, or alpha-chymotrypsin, or any combinations thereof. In any one or more of the embodiments herein, the protease can comprise Liberase™. In any of the embodiments herein, the lipase comprises a pancreatic, hepatic and/or lysosomal lipase, or any combinations thereof. In any one or more of the embodiments herein, the lipase can comprise sphingomyelinase or esterase, or a combination thereof. In any of the embodiments herein, the detergent comprises SDS and the protease comprises proteinase K. In any of the embodiments herein, the detergent and protease are provided in a buffer of at least pH 8.0. In any of the embodiments herein, the biological sample is treated with the detergent and the protease at at least 45° C. for no more than 4 minutes. In any of the embodiments herein, the biological sample is treated with the detergent and the protease at about 50° C. for about 3 minutes. In any of the embodiments herein, the biological sample is treated with 1% SDS and 200 μg/mL proteinase K provided in a PBS buffer of at least pH 8.5 at about 50° C. for about 3 minutes.

In some cases, the detergent comprises SDS and the protease comprises proteinase K. In some embodiments, the method comprises treating the biological sample embedded in the three-dimensional polymerized matrix with 50 to 500 μg/mL proteinase K, 100 to 400 μg/mL proteinase K, 150 to 300 μg/mL proteinase K, or 150 to 250 μg/mL proteinase K. In some embodiments, the method comprises treating the biological sample embedded in the three-dimensional polymerized matrix with about 200 μg/mL proteinase K. In some embodiments, the method comprises treating the biological sample embedded in the three-dimensional polymerized matrix with 0.5% to 2% SDS, 0.5% to 1% SDS, 1% to 2% SDS or about 0.8% to 1.2% SDS. In some embodiments, the method comprises treating the biological sample embedded in the three-dimensional polymerized matrix with 1% SDS. In some embodiments, the method may comprise treating the biological sample embedded in the three-dimensional polymerized matrix with a detergent and a protease in a reaction buffer at about pH 8 to pH 9. In some embodiments, the method may comprise treating the biological sample embedded in the three-dimensional polymerized matrix with a detergent and a protease in a reaction buffer at about pH 8.5.

In some embodiments, the method may comprise treating the biological sample embedded in the three-dimensional polymerized matrix with a detergent and a protease in a reaction buffer at about 45° C. to 60° C., at about 45° C. to 55° C., at about 45° C. to 50° C., at about 48° C. to 55° C., at about 48° C. to 52° C., or at about 50° C. to 52° C. In some embodiments, the method may comprise treating the biological sample embedded in the three-dimensional polymerized matrix with a detergent and a protease in a reaction buffer at about 50° C. for at least 2 minutes, at least 3 minutes, or at least 4 minutes. In some embodiments, the method may comprise treating the biological sample embedded in the three-dimensional polymerized matrix with a detergent and a protease in a reaction buffer at about 50° C. for no more than 5 minutes, no more than 4 minutes, or no more than 3 minutes. In some instances, the biological sample embedded in the three-dimensional polymerized matrix is treated with 1% SDS and 200 μg/ml PK in PBS pH 8.5 at 50° C. for about 3 minutes.

In any of the embodiments herein, the method can comprise contacting the biological sample with a probe or probe set that binds directly or indirectly to the ribonucleic acid. In any one or more of the embodiments herein, the probe or probe set can comprise a detectable probe. In any one or more of the embodiments herein, the probe or probe set can comprise a detectably labeled probe. In any one or more of the embodiments herein, the probe or probe set can comprise a fluorescently labeled probe. In any one or more of the embodiments herein, the probe or probe set can comprise a detectable sequence such as a barcode sequence. In any one or more of the embodiments herein, the probe or probe set can comprise a detectable sequence, and one or more detectably labeled probes (e.g., one or more fluorescently labeled probes) configured to directly or indirectly bind to the detectable sequence or a complement thereof can be used to detect the probe or probe set and the associated ribonucleic acid. In any one or more of the embodiments herein, signals associated with the one or more detectably labeled probes and/or absence of the signals at locations in the biological sample can be analyzed to detect the ribonucleic acid at the locations in the biological sample. In some embodiments, the biological sample is contacted with the probe or probe set after clearing the biological sample embedded in the three-dimensional polymerized matrix.

In any of the embodiments herein, the probe or probe set can be a circular or circularizable probe or probe set. In any of the embodiments herein, the method can comprise circularizing the circularizable probe or probe set using the ribonucleic acid or a product thereof as a template. In any of the embodiments herein, the method can comprise generating an RCA product using the circular or circularizable probe as a template. In any of the embodiments herein, the method can comprise imaging the biological sample to detect the probe or probe set or the RCA product. In any of the embodiments herein, imaging the biological sample can comprise detecting a signal associated with the probe or probe set or the RCA product. In some embodiments, the signal is from a fluorescently labeled probe that directly or indirectly binds to the probe or probe set or the RCA product. In some embodiments, the method comprises providing a fluorescently labeled probe that directly or indirectly binds to the probe or probe set or the RCA product, and imaging the biological sample can comprise detecting a signal from the fluorescently labeled probe that directly or indirectly binds to the probe or probe set or the RCA product. In any of the embodiments herein, the probe or probe set can comprise a barcode sequence. In any of the embodiments herein, the method can comprise detecting the barcode sequence or a complement thereof in the probe or probe set or in a product of the probe or probe set.

In another aspect, provided herein is a method of analyzing a fragmented ribonucleic acid in a biological sample, the method comprising: (a) contacting the biological sample comprising a fragmented ribonucleic acid with a 3′ phosphatase to provide a fragmented ribonucleic acid comprising a 2′,3′-vicinal diol; (b) contacting the biological sample with formylation reagent, wherein the formylation reagent converts the 2′,3′-vicinal diol moiety into 2′3′-dialdehyde moiety; (c) contacting the biological sample with an attachment agent comprising at least one aldehyde-reactive group capable of reacting with at least one aldehyde of the 2′,3′-dialdehyde moiety of the fragmented ribonucleic acid to form a covalent bond and a second reactive group capable of reacting with a matrix-forming agent to form a covalent bond; (d) contacting the biological sample with a matrix-forming agent; (e) forming a three-dimensional polymerized matrix from the matrix-forming agent, thereby embedding the biological sample in the three-dimensional polymerized matrix and anchoring the fragmented ribonucleic acid to the three-dimensional polymerized matrix; (f) clearing the biological sample; (g) contacting the biological sample with a probe or probe set that binds directly or indirectly to the ribonucleic acid; and (h) detecting the probe or a product thereof at a location in the matrix.

In any of the embodiments herein, the fragmented ribonucleic acid in step (a) can have a 2′,3′cyclo-phosphate fragmentation at the 3′-terminal end or a 2′ hydroxyl and a 3′ phosphate fragmentation at the 3′-terminal end, and the 3′ phosphatase can catalyze the formation of the 2′,3′-vicinal diol. In any of the embodiments herein, the 3′ phosphatase can be T4 polynucleotide kinase. In any of the embodiments herein, the fragmented ribonucleic acid of step (a) can be generated by treating the biological sample with a degradation agent to induce fragmentation of ribonucleic acids. In some embodiments, the degradation agent can be an RNase or restriction enzyme. In any of the embodiments herein, the fragmented ribonucleic acid can further comprise an additional vicinal diol moiety provided by a 5′ cap. In some embodiments, the 5′ cap may be a 7-methylguanosine cap. In any of the embodiments herein, the formylation reagent can be sodium (meta)periodate. In any of the embodiments herein, the attachment agent can be N-(2-aminoethyl)methacrylamide, 2-aminoethyl methacrylate, or 2-aminoethyl (E)-but-2-enoate. In any of the embodiments herein, the attachment agent can be N-(2-aminoethyl)methacrylamide. In any of the embodiments herein, the attachment agent can be 2-aminoethyl methacrylate. In any of the embodiments herein, the attachment agent can be 2-aminoethyl (E)-but-2-enoate. In any of the embodiments herein, the step of contacting the biological sample and attachment agent can further comprise contacting the biological sample with a reducing agent. In some embodiments, the reducing agent can be sodium borohydride.

In any of the embodiments herein, the biological sample can be cleared with a detergent, a lipase, and/or a protease. In any of the embodiments herein, the detergent can comprise a non-ionic surfactant or anionic surfactant. In some embodiments, the detergent comprises SDS, tergitol, NP-40, saponin, polyethylene glycol p-(1,1,3,3-tetramethylbutyl)-phenyl ether, or polysorbate 20, or any combinations thereof. In any of the embodiments herein, the protease can comprise proteinase K, pepsin, or collagenase, trypsin, dispase, thermolysin, or alpha-chymotrypsin, or any combinations thereof. In some embodiments, the protease comprises Liberase™. In any of the embodiments herein, the lipase can comprise a pancreatic, hepatic and/or lysosomal lipase, or any combinations thereof. In some embodiments, the lipase comprises sphingomyelinase or esterase, or a combination thereof.

In any of the embodiments herein, the probe or probe set can be a circular or circularizable probe or probe set capable of binding the fragmented ribonucleic acid. In any of the embodiments herein, the method can comprise generating a rolling circle amplification (RCA) product of the circular or circularizable probe or probe set; and detecting a signal associated with a fluorescently labeled probe that directly or indirectly binds to the rolling circle amplification product.

In yet another aspect, provided herein is a method of analyzing a biological sample, the method comprising: (a) contacting the biological sample comprising fragmented ribonucleic acid with T4 polynucleotide kinase, wherein the T4 polynucleotide kinase catalyzes formation of a 2′,3′-vicinal diol moiety on the fragmented ribonucleic acid; (b) contacting the biological sample with sodium (meta)periodate, and wherein the sodium (meta)periodate converts the 2′,3′-vicinal diol to a 2′3′-dialdehyde moiety; (c) contacting the biological sample with N-(2-aminoethyl)methacrylamide, 2-aminoethyl methacrylate, or 2-aminoethyl (E)-but-2-enoate and sodium borohydride, wherein the N-(2-aminoethyl)methacrylamide, 2-aminoethyl methacrylate, or 2-aminoethyl (E)-but-2-enoate reacts with at least one aldehyde of the 2′3′-dialdehyde moiety of the ribonucleic acid to form 3′-aminoethylene-methacrylamide, 3′-aminoethylene-methacrylate, or 3′-aminoethyl (E)-but-2-enoate; (d) contacting the biological sample with a matrix-forming agent; (e) forming a polymerized matrix from the matrix-forming agent, thereby embedding the biological sample in the three-dimensional polymerized matrix and anchoring the fragmented ribonucleic acid to the three-dimensional polymerized matrix; (f) clearing the biological sample; (g) contacting the biological sample with a circular or circularizable probe or probe set, wherein the circular or circularizable probe binds the ribonucleic acid; (h) generating a rolling circle amplification (RCA) product of the circular or circularizable probe or probe set; and (i) detecting a signal associated with a fluorescently labeled probe that directly or indirectly binds to the rolling circle amplification product. In any of the embodiments herein, the attachment agent can be -(2-aminoethyl)methacrylamide and the -(2-aminoethyl)methacrylamide reacts with at least one aldehyde of the 2′3′-dialdehyde moiety of the ribonucleic acid to form 3′-aminoethylene-methacrylamide. In any of the embodiments herein, the attachment agent can be 2-aminoethyl methacrylate and the 2-aminoethyl methacrylate reacts with at least one aldehyde of the 2′3′-dialdehyde moiety of the ribonucleic acid to form 3′-aminoethylene-methacrylate. In any of the embodiments herein, the attachment agent can be 2-aminoethyl (E)-but-2-enoate and the 2-aminoethyl (E)-but-2-enoate reacts with at least one aldehyde of the 2′3′-dialdehyde moiety of the ribonucleic acid to form 3′-aminoethyl (E)-but-2-enoate.

In any of the embodiments herein, the biological sample can comprise cells or cellular components. In any of the embodiments herein, the biological sample can be a tissue sample. In any of the embodiments herein, the tissue sample can be a tissue slice between about 1 μm and about 50 μm in thickness. In any of the embodiments herein, the biological sample can be fixed. In any of the embodiments herein, the biological sample can be a formalin-fixed, paraffin-embedded (FFPE) sample, a fresh tissue sample, or a frozen tissue sample. In any of the embodiments herein, the methods can comprise staining, permeabilizing, cross-linking, expanding, and/or de-cross-linking the biological sample embedded in the three-dimensional polymerized matrix. In any of the embodiments herein, the biological sample and fragmented ribonucleic acid can be treated with a ribonuclease inhibitor. In any of the embodiments herein, the step of contacting the biological sample with the 3′ phosphatase may be performed in the absence of added ammonium ions, phosphate ions, or metal chelators (e.g., the contacting can be performed in the absence of ammonium ions, phosphate ions, or metal chelators that are exogenous to the biological sample). In any of the embodiments herein, the step of contacting the biological sample with the 3′ phosphatase may be performed without sodium chloride or potassium chloride buffer having a concentration of greater than 50 mM. In any of the embodiments herein, the step of contacting the biological sample with the 3′ phosphatase may be performed with a buffer having a sodium chloride or potassium chloride concentration of less than 50 mM. In any of the embodiments herein, the step of contacting the biological sample with the 3′ phosphatase may be performed with a buffer having ions and/or ion concentrations that do not interfere with the 3′ phosphatase activity of the phosphatase, such as T4 polynucleotide kinase (PNK) which is a bifunctional 5′-kinase/3′-phosphatase. In any of the embodiments herein, the matrix-forming agent can comprise acrylamide, bisacrylamide, cellulose, alginate, polyamide, agarose, dextran, or polyethylene glycol. In any of the embodiments herein, the matrix can comprise polyacrylamide, cellulose, alginate, polyamide, cross-linked agarose, cross-linked dextran or cross-linked polyethylene glycol. In any of the embodiments herein, the three-dimensional polymerized matrix can be formed by subjecting the matrix-forming agent to polymerization (or to further polymerization, in the case of matrix-forming agents that are polymers such as polyethylene glycol). In any of the embodiments herein, the polymerization can be initiated by adding a polymerization-inducing catalyst, UV light or functional cross-linkers. In any of the embodiments herein, the fragmented ribonucleic acid can be fragmented mRNA.

In another aspect, provided herein is a kit for analyzing fragmented nucleic acids in a biological sample, comprising: (a) a 3′ phosphatase, optionally wherein the 3′ phosphatase is T4 polynucleotide kinase; (b) a formylation reagent; (c) an attachment agent comprising at least one aldehyde-reactive group capable of reacting with an aldehyde moiety and at least one attachment moiety capable of attaching covalently or non-covalently to an exogenous or endogenous molecule in the biological sample; (d) an exogenous molecule capable of attaching covalently or non-covalently to the attachment moiety; (e) a clearing agent; and (f) instructions for use. In any one or more of the embodiments herein, the formylation reagent can comprise an oxidant. In any one or more of the embodiments herein, the oxidant can comprise a sodium metaperiodate. In any one or more of the embodiments herein, the kit can further comprise one or more reagents for reacting the attachment agent with the aldehyde. In any one or more of the embodiments herein, the exogenous molecule can comprise a matrix-forming agent for embedding the biological sample in a three-dimensional polymerized matrix. In any one or more of the embodiments herein, the clearing agent can comprise a detergent, a lipase, and/or a protease.

### BRIEF DESCRIPTION OF THE DRAWINGS

The drawings illustrate certain features and advantages of this disclosure. These embodiments are not intended to limit the scope of the appended claims in any manner.

FIGS. 1A-1C depict generalized schemes for immobilizing fragmented ribonucleic acids to an endogenous or exogenous molecule in a biological sample.

FIGS. 2A-2B illustrate two exemplary mechanisms for ribonucleic acid fragmentation in in vitro biological samples.

FIG. 3 depicts an exemplary 5′ methylguanosine cap containing two vicinal diols.

FIGS. 4A-4B depict exemplary schemes according to the present disclosure for modifying ribonucleic acids with exemplary attachment agents.

FIG. 5 depicts an exemplary scheme for according to the present disclosure for modifying a ribonucleic acid with an exemplary attachment agent.

FIG. 6 depicts median transcripts per cell in formalin-fixed paraffin-embedded (FFPE) human tissue microarray (TMA) that were embedded in hydrogel (H) or not embedded in hydrogel (NH). TMA samples included brain, lung, skin, colon, pancreas, liver and kidney samples.

FIG. 7 depicts the fraction of transcripts decoded and assigned to a cell in formalin-fixed paraffin-embedded (FFPE) human tissue microarray (TMA) that were embedded in hydrogel (H) or not embedded in hydrogel (NH). TMA samples included brain, lung, skin, colon, pancreas, liver and kidney samples.

FIG. 8 depicts total decoded transcripts in formalin-fixed paraffin-embedded (FFPE) human tissue microarray (TMA) that were embedded in hydrogel (H) or not embedded in hydrogel (NH). TMA samples included brain, lung, skin, colon, pancreas, liver and kidney samples.

FIG. 9 depicts gene localization in a human skin sample that was embedded in hydrogel (H) or not embedded in hydrogel (NH).

FIG. 10 depicts gene localization in a human pancreas sample that was embedded in hydrogel (H) or not embedded in hydrogel (NH).

FIG. 11 depicts gene localization in a human liver sample that was embedded in hydrogel (H) or not embedded in hydrogel (NH).

FIG. 12 shows an image depicting differences in RCP density in a human liver sample that was embedded in hydrogel (H) or not embedded in hydrogel (NH). Top panel: RCPs in liver sample (white dots). Bottom panel: nuclei in liver sample.

FIG. 13 depicts RCPs in formalin-fixed paraffin-embedded (FFPE) mouse brain (mBrain) tissue samples treated with various conditions.

FIG. 14 depicts the total number of RCPs in formalin-fixed paraffin-embedded (FFPE) mouse brain (mBrain) tissue samples treated with various conditions at various wavelengths including green, yellow, orange, and red.

FIG. 15 depicts spatial resolution of RCPs in formalin-fixed paraffin-embedded (FFPE) mouse brain (mBrain) tissue samples treated with various conditions at various wavelengths including green, yellow, orange, and red. Spatial resolution is depicted as full width at half maximum (FWHM

FIG. 16 depicts local signal-to-background ratio in formalin-fixed paraffin-embedded (FFPE) mouse brain (mBrain) tissue samples treated with various conditions at various wavelengths including green, yellow, orange, and red.

### DETAILED DESCRIPTION

All publications, comprising patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Overview

Provided herein are methods and kits for analyzing ribonucleic acids (RNA), particularly highly degraded or fragmented RNA, in a biological sample (e.g., tissue). The methods and kits provided herein can be applied to various applications such as in situ methods. In situ analysis of the identity and spatial localization of RNA requires positional stability of the RNA. However, the preparation of many samples for in situ analysis undergo several harsh processing steps (e.g., formalin-fixed, paraffin-embedded (FFPE) tissues). These steps include baking and deparaffinization, decrosslinking, and permeabilization. The vast majority of RNA (e.g., mRNA) can be lost during and after the decrosslinking step.

Hydrogel-based approaches to emerging in situ technologies offer certain advantages including reduced background autofluorescence and improved diffusional parameters, as well as enhanced tissue adhesion to the hydrogel and temporally spaced orthogonal chemistries that can be leveraged to couple tissue to the hydrogel. However, most hydrogel-based approaches have notable limitations. For example, pre-embedding a biological sample with hydrogel monomers prior to digestion of cellular components (e.g., proteins and lipids) and amplification is inherently more time consuming and limited in value for samples containing degraded or fragmented RNA. The methods and kits disclosed herein are intended to overcome many of these shortcomings through use of a brief, enzymatic reaction that converts all 3′ ends of fragmented RNA molecules into vicinal diols. The vicinal diols on the converted fragments, and/or present on any non-fragmented RNA molecule(s) as well, can be further modified to convert the diols into aldehydes. At this point, reactive groups can be covalently attached to the 3′ end of fragmented RNA for incorporation into the hydrogel. Because the 3′ end of the majority of fragmented RNA can be converted into vicinal diols and unfragmented RNA naturally contains vicinal diols at the 3′ end, the present disclosure provides a substrate that can be broadly leveraged for hydrogel-based in situ applications. The methods involving enzymatic and chemical reactions provided herein can enable pre-embedding approaches to samples that include degraded RNA, preserving their spatial orientation and opening access to thousands of RNA fragments/cell that have been unable to study until now. In some aspects, the tethering methods provided herein are agnostic to the size of RNA fragments (any 3′ end can be tethered) and can be used with compromised biological samples. For example, RNA fragments of about 10 to 100 nucleotides, about 10 to 80 nucleotides, about 10 to 60 nucleotides, about 10 to 40 nucleotides, about 10 to 20 nucleotides, about 20 to 100 nucleotides, about 20 to 80 nucleotides, about 20 to 60 nucleotides, about 20 to 40 nucleotides, or about 20 to 30 nucleotides are analyzed using the methods provided herein. In some embodiments, RNA fragments of about 20 to 50 nucleotides are analyzed using the methods provided herein. In some embodiments, 3′ end tethering of fragmented RNA can increase sensitivity and reduce the need for using multiple probes per analyte.

### II. Methods for Analyzing RNA

Provided herein are methods for analyzing RNA in a biological sample (e.g., a tissue sample). The methods provided herein include a series of enzymatic and non-enzymatic reactions that can be utilized to immobilize or tether any fragmented ribonucleic acids to an endogenous molecule in the biological sample or an exogenous molecule delivered to the biological sample, such as a matrix-forming agent.

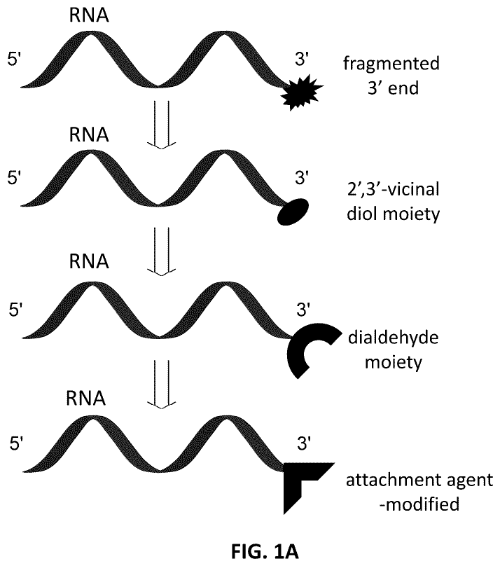

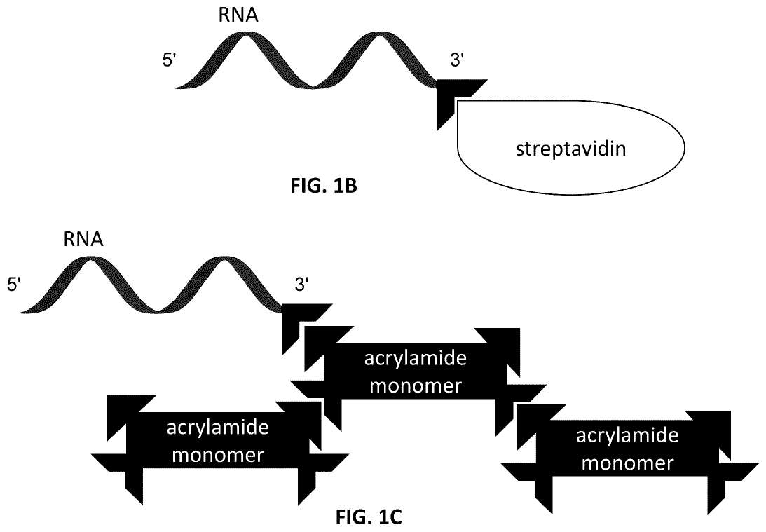

More specifically, the methods and kits as provided herein enable conversion of fragmented 3′ ends of RNA to functional groups capable of being immobilized in the biological sample. With reference to FIG. 1A, an exemplary scheme according to the methods of the present disclosure is shown. In FIG. 1A, a ribonucleic acid having a fragmented terminal 3′ end is converted to 2′,3′ vicinal diol followed by formylation to provide an aldehyde moiety. The aldehyde moiety is further modified chemically with an attachment agent that allows the RNA to bond covalently or bind non-covalently to a molecule in the biological sample. As shown in FIGS. 1B and 1C, the attachment agent may be selected for bonding to an endogenous molecule (e.g., streptavidin) or an exogenous molecule (e.g., matrix-forming agents, such as acrylamide monomers). Examples of matrix-forming agents include but are not limited to acrylamide, bisacrylamide, cellulose, alginate, polyamide, agarose, dextran, and polyethylene glycol.

In one aspect, provided herein is a method, comprising: (a) contacting a biological sample comprising a ribonucleic acid with a formylation reagent, wherein the ribonucleic acid comprises a 2′,3′-vicinal diol and the formylation reagent converts the 2′,3′-vicinal diol moiety into a 2′3′-dialdehyde moiety; and (b) contacting the biological sample with an attachment agent comprising at least one aldehyde reactive group (e.g., a first reactive group) capable of reacting with at least one aldehyde of the 2′,3′-dialdehyde moiety of the ribonucleic acid to form a covalent bond and at least one attachment moiety capable of attaching covalently or non-covalently to an exogenous or endogenous molecule in the biological sample, thereby immobilizing the ribonucleic acid in the biological sample.

In some embodiments, the exogenous or endogenous molecule in the biological sample is present in the biological sample throughout the method of the present disclosure. Alternatively, in some embodiments the method of the present disclosure comprises introducing the exogenous or endogenous molecule into the biological sample. Thus, the method of the present disclosure is not limited to methods in which the exogenous or endogenous molecule is present in the biological sample prior to contacting the biological sample with a formylation reagent, or prior to contacting the biological sample with the attachment agent. When the method of the present disclosure comprises introducing the exogenous or endogenous molecule into the biological sample, the exogenous or endogenous molecule is typically introduced into the biological sample at any stage prior to immobilizing the ribonucleic acid in the biological sample.

In some embodiments of the method of the present disclosure, the aldehyde reactive group typically reacts with at least one aldehyde of the 2′,3′-dialdehyde moiety of the ribonucleic acid to form a covalent bond, and the attachment moiety typically attaches covalently or non-covalently to an exogenous or endogenous molecule.

In another aspect, provided herein is a method of analyzing a fragmented ribonucleic acid in a biological sample, the method comprising: (a) contacting the biological sample comprising a fragmented ribonucleic acid with a 3′ phosphatase to provide a fragmented ribonucleic acid comprising a 2′,3′-vicinal diol; (b) contacting the biological sample with formylation reagent, wherein the formylation reagent converts the 2′,3′-vicinal diol moiety into 2′3′-dialdehyde moiety; (c) contacting the biological sample with an attachment agent comprising at least one aldehyde reactive group (e.g., a first reactive group) capable of reacting with at least one aldehyde of the 2′,3′-dialdehyde moiety of the fragmented ribonucleic acid to form a covalent bond and an attachment moiety (e.g., a second reactive group) capable of reacting with a matrix-forming agent to form a covalent bond; (d) contacting the biological sample with a matrix-forming agent; (e) forming a three-dimensional polymerized matrix from the matrix-forming agent, thereby embedding the biological sample in the three-dimensional polymerized matrix and anchoring the fragmented ribonucleic acid to the three-dimensional polymerized matrix; (f) clearing the biological sample; (g) contacting the biological sample with a probe or probe set that binds directly or indirectly to the ribonucleic acid; and (h) detecting the probe or a product thereof at a location in the matrix.

In some embodiments of the method of analyzing a fragmented ribonucleic acid in a biological sample of the present disclosure, the aldehyde reactive group typically reacts with at least one aldehyde of the 2′,3′-dialdehyde moiety to form a covalent bond, and the attachment moiety typically reacts with the matrix-forming agent to form a covalent bond.

In yet another aspect, provided herein is a method of analyzing a biological sample, the method comprising: (a) contacting the biological sample comprising fragmented ribonucleic acid with T4 polynucleotide kinase, wherein the T4 polynucleotide kinase catalyzes formation of a 2′,3′-vicinal diol moiety on the fragmented ribonucleic acid; (b) contacting the biological sample with sodium (meta)periodate, and wherein the sodium (meta)periodate converts the 2′,3′-vicinal diol to a 2′3′-dialdehyde moiety; (c) contacting the biological sample with N-(2-aminoethyl)methacrylamide, 2-aminoethyl methacrylate, or 2-aminoethyl (E)-but-2-enoate and sodium borohydride, wherein the N-(2-aminoethyl)methacrylamide, 2-aminoethyl methacrylate, or 2-aminoethyl (E)-but-2-enoate reacts with at least one aldehyde of the 2′3′-dialdehyde moiety of the ribonucleic acid to form 3′-aminoethylene-methacrylamide, 3′-aminoethylene-methacrylate, or 3′-aminoethyl (E)-but-2-enoate; (d) contacting the biological sample with a matrix-forming agent; (e) forming a polymerized matrix from the matrix-forming agent, thereby embedding the biological sample in the three-dimensional polymerized matrix and anchoring the fragmented ribonucleic acid to the three-dimensional polymerized matrix; (f) clearing the biological sample; (g) contacting the biological sample with a circular or circularizable probe or probe set, wherein the circular or circularizable probe binds the ribonucleic acid; (h) generating a rolling circle amplification (RCA) product of the circular or circularizable probe or probe set; and (i) detecting a signal associated with a fluorescently labeled probe that directly or indirectly binds to the rolling circle amplification product.

In some embodiments of the method of analyzing a biological sample of the present disclosure, the 3′-aminoethylene-methacrylamide, 3′-aminoethylene-methacrylate, or 3′-aminoethyl (E)-but-2-enoate typically reacts with the matrix-forming agent to form a covalent bond.

In a further aspect, provided herein is a method of analyzing a biological sample comprising a fragmented ribonucleic acid (RNA), the method comprising: (a) contacting the biological sample comprising the fragmented RNA with T4 polynucleotide kinase, wherein the T4 polynucleotide kinase catalyzes formation of a 2′,3′-vicinal diol moiety on the fragmented RNA; (b) contacting the biological sample with a deglycosylation agent; (c) contacting the biological sample with a matrix-forming agent, wherein the matrix-forming agent comprises a boronic acid moiety capable of covalently reacting with at least one 2′,3′ vicinal diol of the RNA, and wherein the biological sample and the matrix-forming agent are contacted under conditions suitable to form a covalent bond between the boronic acid moiety and the 2′,3;-vicinal diol of the RNA; (d) forming a three-dimensional polymerized matrix from the matrix-forming agent, thereby embedding the biological sample in the three-dimensional polymerized matrix and anchoring the fragmented RNA to the three-dimensional polymerized matrix; (e) contacting the biological sample with a detergent and a protease; (f) contacting the biological sample with a probe or probe set that binds directly or indirectly to the RNA; and (g) detecting the probe or a product thereof at a location in the matrix. In some instances, the detergent and protease are provided in a buffer of at least pH 8.5. In some instances, the detergent and protease are contacted with the sample for no more than 4 minutes at at least 50° C.

In some embodiments, the T4 polynucleotide kinase polishes the 3′ RNA ends into vicinal diols, NaIO₄ oxidizes the diols into aldehydes, 2-AEM uses the amino group to react with the aldehydes, adding 2 methacrylamide groups to all RNA fragments (including e.g., mRNA, 1ncRNA, miRNA). In some cases, aniline can be added as a catalyst and may help imine formation. In some cases, the resulting imine linkage to the RNA can be readily hydrolyzed. In some embodiments, the sample is treated including reduction with NaBH₄ to make the processing irreversible. In some embodiments, the NaBH₄ is suspended in water and treated with the sample. In some embodiments, the NaBH₄ is suspended in ethanol and treated with the sample.

### A. Ribonucleic Acid(s)

In some embodiments, the RNA analyzed by a method provided herein comprises various types of coding and non-coding RNA. Examples of the different types of RNA analytes include messenger RNA (mRNA), including a nascent RNA, a pre-mRNA, a primary-transcript RNA, and a processed RNA, such as a capped mRNA (e.g., with a 5′ 7-methyl guanosine cap), a polyadenylated mRNA (poly-A tail at the 3′ end), and a spliced mRNA in which one or more introns have been removed. Also included in the analytes disclosed herein are non-capped mRNA, a non-polyadenylated mRNA, and a non-spliced mRNA. The RNA analyte can be a transcript of another nucleic acid molecule (e.g., DNA or RNA such as viral RNA) present in a tissue sample. Examples of a non-coding RNAs (ncRNA) that is not translated into a protein include transfer RNAs (tRNAs) and ribosomal RNAs (rRNAs), as well as small non-coding RNAs such as microRNA (miRNA), small interfering RNA (siRNA), Piwi-interacting RNA (piRNA), small nucleolar RNA (snoRNA), small nuclear RNA (snRNA), extracellular RNA (exRNA), small Cajal body-specific RNAs (scaRNAs), and the long ncRNAs such as Xist and HOTAIR. The RNA can be small (e.g., less than 200 nucleic acid bases in length) or large (e.g., RNA greater than 200 nucleic acid bases in length). Examples of small RNAs include 5.8S ribosomal RNA (rRNA), 5S rRNA, tRNA, miRNA, siRNA, snoRNAs, piRNA, tRNA-derived small RNA (tsRNA), and small rDNA-derived RNA (srRNA). The RNA can be double-stranded RNA or single-stranded RNA. The RNA can be circular RNA. The RNA can be a bacterial rRNA (e.g., 16s rRNA or 23s rRNA).

As detailed above, the methods of the present disclosure are especially suitable for immobilization of fragmented RNA. In some embodiments, any one of the RNA analytes disclosed herein can be fragmented. In some embodiments, the RNA analyzed by the methods provided herein comprises a fragmented RNA. In some embodiments, the RNA is a fragmented RNA. In some embodiments, the fragmented RNA is fragmented mRNA. In some embodiments, the fragmented RNA comprises a 2′,3′-vicinal diol. In some embodiments, the 2′,3′-vicinal diol is at the fragmented 3′ end of the RNA. In some embodiments, the 2′,3′-vicinal diol is a fragmented 3′ end of the RNA.

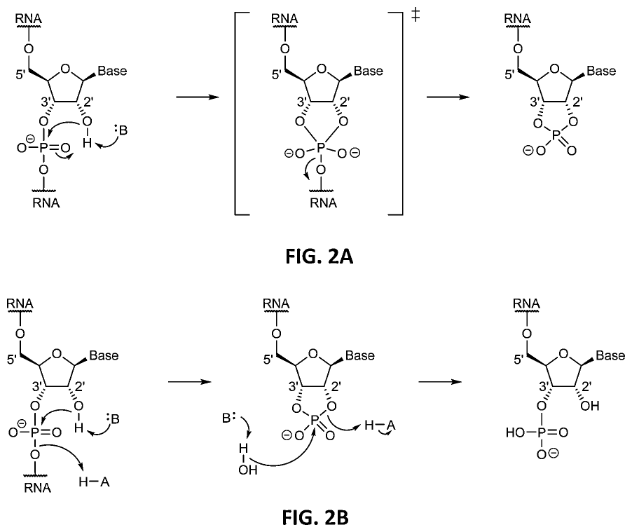

In FIGS. 2A and 2B, exemplary mechanisms for ribonucleic acid fragmentation in situ are shown. With reference to FIG. 2A, RNA fragmentation can result in formation of 2′,3′-cyclo-phosphate on the fragmented 3′ end of the ribonucleic acid. With reference to FIG. 2B, a separate fragmentation mechanism results in an RNA fragment having a 2′ hydroxyl group and 3′ phosphate group on the terminal 3′ end of the RNA. The two mechanisms illustrated in FIGS. 2A and 2B are two of the most common fragmentation patterns observed for RNA in biological samples. As provided herein, the methods of the present disclosure utilize enzymatic reactions, driven by 3′-phosphatases, to convert these most commonly occurring RNA fragments into a 2′,3′-vicinal diol.

In some embodiments, the 2′,3′-vicinal diol is generated from a fragmented RNA having a 2′,3′cyclo-phosphate fragmentation at the 3′-terminal end or a 2′ hydroxyl and a 3′ phosphate fragmentation at the 3′-terminal end. In some embodiments, the 2′,3′-vicinal diol is generated from a fragmented RNA having a 2′,3′cyclo-phosphate fragmentation at the 3′-terminal end. In some embodiments, the 2′,3′-vicinal diol is generated from a fragmented RNA having a 2′ hydroxyl and a 3′ phosphate fragmentation at the 3′-terminal end. In some embodiments, the 2′,3′-vicinal diol is provided by contacting a fragmented RNA with a 3′ phosphatase. In some embodiments, the methods provided herein comprise contacting a fragmented RNA, wherein the fragmented RNA comprises a 2′,3′cyclo-phosphate fragmentation at the 3′-terminal end or a 2′ hydroxyl and a 3′ phosphate fragmentation at the 3′-terminal end, with a 3′-phosphatase to generate a fragmented RNA comprising a 2′,3′-vicinal diol.

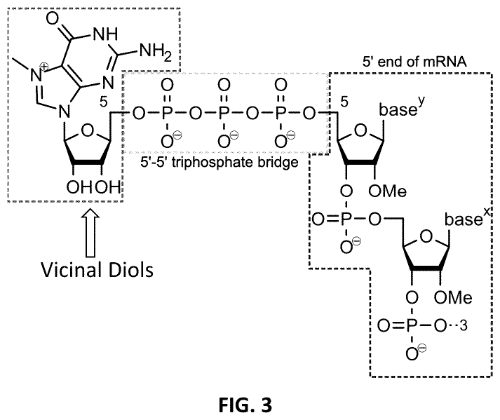

The methods of the present disclosure may also be extended to direct immobilization of any RNA analytes, whether fragmented or not, wherein the RNA analyte possesses a vicinal diol moiety, for example, on a 5′ cap or an intact 3′ terminal end. An exemplary 5′ cap, 7-methylguanosine, is shown in FIG. 3. In some embodiments, the RNA comprising a 2′,3′-vicinal diol comprises a 5′ cap and the 2′,3′-vicinal diol is in the 5′ cap. In some embodiments, the 5′ cap is a 7-methylguanosine cap.

In some embodiments, the fragmented ribonucleic acid is generated by treating the biological sample containing the ribonucleic acid with a degradation agent to induce fragmentation of ribonucleic acids. The term “degradation agent” refers to a chemical reagent or enzyme that induces fragmentation of RNA. Examples of degradation agents include a nuclease, like a ribonuclease, or a restriction enzyme. In certain embodiments, the degradation agent is an RNase or restriction enzyme.

Ribonucleases, or RNases, are nucleases that catalyze degradation of RNA. Exemplary RNases may include but are not limited to RNase A, RNase B, RNase C, RNase E, RNase H, RNase HI, RNase HII, RNase II, RNase III, RNase F1, RNase L, RNase M, RNase Ms, RNase N, RNase P, RNase PhyM, RNase R, RNase Sa, RNase St, RNase T1, RNase T2, RNase U2, RNase IV, RNase V, RNase E, RNase E, polynucleotide phosphorylase (PNPase), RNase PH, RNase, RNase BN, RNase D, RNase T, RNase 1, oligoribonuclease, exoribonuclease I, and exoribonuclease II.

A restriction enzyme, restriction endonuclease, REase, ENase or restrictase is an enzyme that cleaves RNA or DNA into fragments at or near specific recognition sites within molecules known as restriction sites. Exemplary restriction enzymes may include but are not limited to EcoRI, EcoRII, BamHI, HindIII, TaqI, NotI, HinFI, Sau3AI, PvuII, SmaI, HaeIII, HgaI, AluI, EcoRV, EcoP15I, KpnI, Pmel, PstI, SacI, SalI, ScaI, SpeI, SphI, StuI, and XbaI.

It should be recognized, however, that in some embodiments, wherein the intentional fragmentation has already been achieved via contact with a degradation agent and/or wherein no further fragmentation is desired, such as for FFPE tissue samples, the methods of the present disclosure may encompass the use of ribonuclease inhibitors. In some embodiments, the RNA is not fragmented RNA.

The methods as described herein may further comprise contacting or treating the biological sample and/or fragmented RNA with RNase inhibitors to prevent any undesired fragmentation. In some embodiments, the method further comprises the biological sample and fragmented ribonucleic acid are treated with a ribonuclease inhibitor. If the biological sample is contacted with a degradation agent to induce fragmentation, the method may further comprise treating the biological sample and fragmented ribonucleic acid with a ribonuclease inhibitor after being contacted with the degradation agent. In some embodiments, the biological sample and fragmented ribonucleic acid are treated with a ribonuclease inhibitor after the biological sample has been contacted with a degradation agent. In some embodiments, the biological sample is treated with one or more RNase inhibitors. In some embodiments, the one or more RNase inhibitors are the same. In some embodiments, the one or more RNase inhibitors are different.

Exemplary ribonuclease inhibitors may include but are not limited to an anti-RNase antibodies, recombinant enzymes, or non-enzymatic inhibitors.

In some embodiments, the anti-RNase antibody is capable of binding to RNase A, RNase B, RNase C, RNase E, RNase H, RNase HI, RNase HII, RNase II, RNase III, RNase F1, RNase L, RNase M, RNase Ms, RNase N, RNase P, RNase PhyM, RNase R, RNase Sa, RNase St, RNase T1, RNase T2, RNase U2, RNase IV, RNase V, RNase E, RNase E, polynucleotide phosphorylase (PNPase), RNase PH, RNase, RNase BN, RNase D, RNase T, RNase 1, oligoribonuclease, exoribonuclease I, or exoribonuclease II. In some embodiments, the anti-RNase antibody comprises the Roche Protector RNase inhibitor (Millipore Sigma, Cat. #C756R82), In some embodiments, the anti-RNase antibody is Roche Protector RNase inhibitor.

In some embodiments, the recombinant enzyme is capable of degrading RNase A, RNase B, RNase C, RNase E, RNase H, RNase HI, RNase HII, RNase II, RNase III, RNase F1, RNase L, RNase M, RNase Ms, RNase N, RNase P, RNase PhyM, RNase R, RNase Sa, RNase St, RNase T1, RNase T2, RNase U2, RNase IV, RNase V, RNase E, RNase E, polynucleotide phosphorylase (PNPase), RNase PH, RNase, RNase BN, RNase D, RNase T, RNase 1, oligoribonuclease, exoribonuclease I, or exoribonuclease II. In some embodiments, the recombinant enzyme comprises Invitrogen's SUPERase⋅In™ RNase Inhibitor, RNaseOUT™ Recombinant Ribonuclease Inhibitor, RNAsecure™ RNase Inactivation Reagent, or Ambion™ RNase Inhibitor.

In some embodiments, the non-enzymatic ribonuclease inhibitor comprises a ribonucleotide-derived RNase inhibitor or a nonnucleotide RNase inhibitor. In some embodiments, the RNase inhibitor is a vanadium salt. In some embodiments, the ribonucleotide-derived RNase inhibitor comprises one or more ribonucleotide vanadyl complexes (RVC), dinucleotide derivatives of adenosine 5′-pyrophosphate, such as 5′-diphosphoadenosine 3′-phosphate (ppA-3′-p) or 5′-diphosphoadenosine 2′-phosphate (ppA-2′-p), diadenosine derivatives, 3′-N-alkylamino-3′-deoxy-ara-uridines, or ribonucleotide zinc complexes, such as 3′-N-oxyurea-3′-deoxythymidine 5′-phosphate zinc complex. In some embodiments, the nonnucleotide RNase inhibitor comprises 8-amino-5-(4′-hydroxybiphenyl-4-ylazo)naphthalene-2-sulfonate or a catechin, such as epi-gallocatechin-3-gallate.

### B. Phosphatase(s)

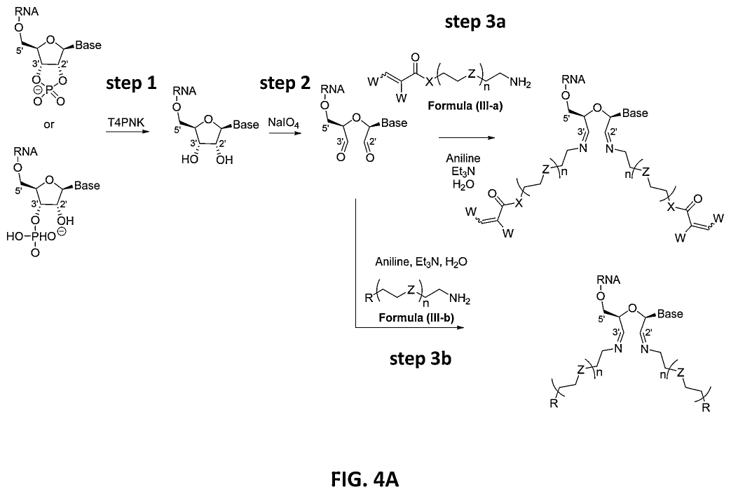

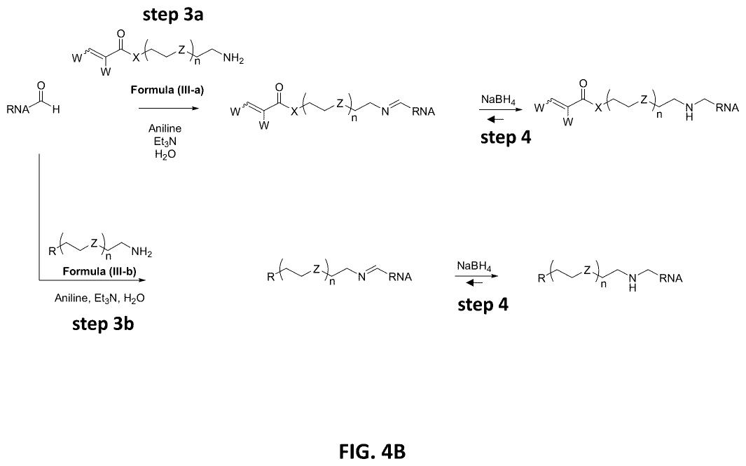

As detailed above, the methods of the present disclosure employ enzymatic conversion of the two most commonly occurring RNA fragmentation patterns into a 2′,3′-vicinal diol moiety, which may be subsequently modified to enable downstream chemistries and immobilization in the biological sample. With reference to FIG. 4A, step 1, exemplary fragmented ribonucleic acids comprising a 2′,3′cyclo-phosphate fragmentation at the 3′-terminal end or a 2′ hydroxyl and a 3′ phosphate fragmentation at the 3′-terminal end may be treated with a 3′ phosphatase, such as T4 polynucleotide kinase, thereby resulting in the formation of a 2′3′-vicinal diol moiety at the 3′-terminal ribose ring. In some embodiments, the 2′,3′-vicinal diol at the 3′ end of the fragmented RNA is generated by a 3′ phosphatase.

In some embodiments, the 2′,3′-vicinal diol is provided by contacting the fragmented ribonucleic acid with a 3′ phosphatase. In some embodiments, the fragmented ribonucleic acid has a 2′,3′cyclo-phosphate fragmentation at the 3′-terminal end or a 2′ hydroxyl and a 3′ phosphate fragmentation at the 3′-terminal end, wherein the 3′ phosphatase catalyzes the formation of the 2′,3′-vicinal diol.

It should be recognized that the 3′ phosphatase as referred to herein may also be referred to as a 3′ nucleotidase or analogous enzyme in the class of hydrolases capable of catalyzing the reaction to convert a ribonucleotide comprising a 3′ terminal phosphate group to the corresponding ribonucleotide and cleaved phosphate. An exemplary listing of 3′ phosphatases is provided in the SIB Swiss Institute of Bioinformatics Expasy enzyme nomenclature database (entry: EC 3.1.3.6). In some embodiments, the 3′ phosphatase comprises a T4 polynucleotide kinase. In some embodiments, the 3′ phosphatase is T4 polynucleotide kinase.

In some embodiments wherein the method comprises contacting a fragmented ribonucleic acid comprising a 2′,3′cyclo-phosphate fragmentation at the 3′-terminal end or a 2′ hydroxyl and a 3′ phosphate fragmentation at the 3′-terminal end with a 3′ phosphatase or analogous enzyme, the step of contacting the biological sample or ribonucleic acid with the 3′ phosphatase is performed under conditions suitable for the 3′ phosphatase activity to occur. For example, in some embodiments, the presence of certain buffers and/or reagents utilized for this step may be limited to those that are compatible with 3′ phosphatase activity or are limited with respect to the concentrations at which they are included to permit 3′ phosphatase activity to occur. In some embodiments, the step of contacting the biological sample with the 3′ phosphatase is not performed in the presence of ammonium ions, phosphate ions, or metal chelators. In some embodiments, the step of contacting the biological sample with the 3′ phosphatase is not performed in the presence of sodium chloride or potassium chloride buffer having a concentration of greater than 50 mM. In any of the embodiments herein, the step of contacting the biological sample with the 3′ phosphatase may be performed with a buffer having a sodium chloride or potassium chloride concentration of less than 50 mM. In any of the embodiments herein, the step of contacting the biological sample with the 3′ phosphatase may be performed with a buffer having ions and/or ion concentrations that do not interfere with the 3′ phosphatase activity of the phosphatase, such as T4 polynucleotide kinase (PNK) which is a bifunctional 5′-kinase/3′-phosphatase.

### C. Formylation Reagent(s)

In some embodiments, the methods of the present disclosure comprise contacting a biological sample comprising a ribonucleic acid with a formylation reagent, wherein the ribonucleic acid comprises a 2′,3′-vicinal diol and the formylation reagent reacts with the 2′,3′-vicinal diol moiety to provide a ribonucleic acid comprising at least one aldehyde moiety, optionally wherein the ribonucleic acid comprises a dialdehyde moiety.

The term “formylation” generally refers to the addition of a formyl or aldehyde group to a chemical or biological entity. As utilized herein, the term “formylation reagent” refers to any suitable chemical and/or biological reagent capable of performing formylation on a ribonucleic acid and, more specifically in the context of the present disclosure, the formylation reagents as provided herein are capable of converting the 2′,3′-vicinal diol on the terminal 3′ end ribose ring of a ribonucleic acid into a ribonucleic acid comprising at least one aldehyde. In some embodiments, the formylation reagent reacts with the 2′,3′-vicinal diol to form at least one aldehyde moiety. In certain embodiments, the formylation reagent is an oxidant, the formylation reagent reacts with the 2′,3′-vicinal diol to form two aldehyde moieties. In step 2 of FIG. 4A, the conversion of a 2′,3′-vicinal diol to the corresponding dialdehyde is illustrated.

As illustrated in FIG. 4A, formylation of the 3′ terminal end of the ribonucleic acid involves opening of the ribose ring at the bond between the carbon atoms labeled as the 2′ and 3′-positions according to standard nomenclature. As provided herein, formylation of the ribonucleic acid entails a ring opening of the ribose ring and the standard nomenclature conventions for carbon numbering in the ribose ring are not strictly applicable to the formylated ribonucleic acid. However, as provided herein, the dialdehyde formed by the formylation agent may still also be referred to as a 2′,3′-dialdehyde moiety, in reference to the positions of the alcohol groups on ribose ring prior to formylation and ring opening. Similarly, the individual aldehydes derived from the alcohol moiety on each carbon atom previously denoted as the 2′ and 3′ ring positions may be referred to as the 2′ aldehyde and 3′ aldehyde, respectively.

In some embodiments, the formylation reagent is a chemical reagent. In some embodiments wherein the formylation reagent is a chemical reagent, the formylation reagent is an oxidant. In some embodiments, the oxidant is sodium (meta)periodate.

In still other embodiments, the step of contacting the biological sample with a formylation reagent to provide a ribonucleic acid having at least one aldehyde may further comprise contacting the biological sample with a ring-opening reagent (e.g., ring opening catalyst) prior to or concomitantly with the formylation reagent.

In other embodiments, the formylation reagent is a biological reagent.

### D. Attachment Agent(s)

As detailed herein, the methods of the present disclosure encompass the preparation of terminal aldehyde moieties from vicinal diols on ribonucleic acids for immobilization of the ribonucleic acids in the biological sample. The methods provided herein achieve immobilization of the ribonucleic acids through the use of an attachment agent that mediates the interaction between the ribonucleic acid and the biological sample. In some embodiments, the methods as provided herein comprise contacting the biological sample with one or more attachment agent. In some embodiments wherein the method comprises contacting the biological sample with two or more attachment agents, the attachment agents may be the same or different.

In some embodiments, the attachment agent is a bi- or multifunctional molecule comprising at least two different functional groups. In some embodiments, the attachment agent is a bifunctional molecule. In some embodiments wherein the attachment agent is a bifunctional molecule, the attachment agent comprises at least two functional groups, the first of which is capable of covalently bonding to the ribonucleic acid and the second of which is capable of covalently or non-covalently bonding to an exogenous or endogenous molecule present in the biological sample. In some variations, the functional group is capable of covalently bonding to the ribonucleic acid by covalently bonding to an aldehyde on the ribonucleic acid, and such a functional group is referred to as an “aldehyde-reactive group”. In some embodiments, the two functional groups of the bifunctional molecule comprise an aldehyde reactive group capable of reacting with at least one aldehyde of the 2′,3′-dialdehyde moiety of the ribonucleic acid to form a covalent and an attachment moiety. In some embodiments, when the attachment agent is a bifunctional molecule, the aldehyde reactive group comprises or is a “first reactive group”. In some embodiments, the two functional groups of the bifunctional molecule comprise or is a first reactive group and an attachment moiety. In some embodiments wherein the attachment agent is a bifunctional molecule, the attachment agent comprises a first reactive group capable of reacting with at least one aldehyde of the 2′,3′-dialdehyde moiety of the ribonucleic acid to form a covalent bond and an attachment moiety capable of attaching covalently or non-covalently to an exogenous or endogenous molecule in the biological sample. In some embodiments wherein the attachment agent is a bifunctional molecule and the attachment moiety is capable of attaching covalently to an exogenous or endogenous molecule in the biological sample, the attachment moiety may be used interchangeably with “second reactive group”. In some embodiments, the second reactive group is capable of attaching covalently to an exogenous or endogenous molecule in the biological sample.

In other embodiments, the attachment agent is a multifunctional molecule. In some embodiments wherein the attachment agent is a multifunctional molecule, the attachment agent comprises at least three (e.g., at least any of 3, 4, 5, 6, 7, or 8) functional groups, wherein at least two functional groups are capable of covalently bonding to the ribonucleic acid and at least one functional group is capable of covalently or non-covalently bonding to an exogenous or endogenous molecule present in the biological sample, or wherein at least two functional groups are capable of covalently or non-covalently bonding to an exogenous or endogenous molecule present in the biological sample and at least one functional group is capable of covalently bonding to the ribonucleic acid. In some embodiments wherein the attachment agent is a multifunctional molecule, the attachment agent may comprise two or more functional groups of the same category, e.g., capable of covalently bonding to the ribonucleic acid or capable of covalently or non-covalently bonding to an exogenous or endogenous molecule present in the biological sample. For example, in some embodiments wherein the attachment agent is a multifunctional molecule, the attachment agent may comprise at least two functional groups capable of covalently bonding to the ribonucleic acid. In other embodiments wherein the attachment agent is a multifunctional molecule, the attachment agent may comprise at least two functional groups capable of covalently or non-covalently bonding to an exogenous or endogenous molecule present in the biological sample. In certain embodiments wherein the attachment agent is a multifunctional molecule, the attachment agent may comprise at least two functional groups capable of covalently bonding to the ribonucleic acid and at least two functional groups capable of covalently or non-covalently bonding to an exogenous or endogenous molecule present in the biological sample. As described herein, the two categories of functional groups of the multifunctional molecule comprise at least an aldehyde reactive group and an attachment moiety.

It should be recognized that reference to the attachment agent, whether bi- or multifunctional, as defined herein refers to the attachment agent prior to binding with the ribonucleic acid and the exogenous or endogenous molecule present in the biological sample, unless otherwise noted.

Whether bifunctional or multifunctional, the attachment agents as provided herein each possess at least one functional group capable of covalently bonding to the ribonucleic acid and at least one functional group capable of covalently or non-covalently bonding to an exogenous or endogenous molecule present in the biological sample. It should be recognized that the multifunctional attachment agent will have at least one additional functional group capable of covalently bonding to the ribonucleic acid or capable of covalently or non-covalently bonding to an exogenous or endogenous molecule present in the biological sample.

i. Aldehyde-Reactive Group or First Reactive Group

As provided herein, the functional group(s) capable of covalently bonding to the ribonucleic acid may can be any reactive group that reacts with and covalently bonds to an aldehyde. In some variations, such functional group(s) capable of covalently bonding to the ribonucleic acid by covalently bonding to an aldehyde on the ribonucleic acid are referred to as “aldehyde-reactive group(s)”. It should be recognized that, in instances wherein the attachment agent is a bifunctional molecule or wherein the attachment agent comprises a single aldehyde-reactive group, the aldehyde-reactive group of the bifunctional attachment agent may be referred to as a “first reactive group”. In some embodiments, the attachment agent comprises an aldehyde-reactive group (or first reactive group in a bifunctional attachment agent) capable of reacting with at least one aldehyde of the 2′,3′-dialdehyde moiety of the ribonucleic acid to form a covalent bond.

In some embodiments, the aldehyde-reactive group or first reactive group comprises or is a nucleophilic group capable of reacting with at least one aldehyde of the 2′,3′-dialdehyde moiety of the ribonucleic acid. In some embodiments, the reactive group comprises or is an amine moiety, an amide moiety, an alcohol moiety, a thiol moiety, a cyano moiety, an ylide moiety, a hydrazide, a hydroxylamine, a hydrazine, a thiosemicarbazone, a hydrazine carboxylate, or an arylhydrazide, or any combination thereof. In some embodiments, the aldehyde-reactive group or first reactive group of the attachment agent is or comprises an amine moiety, an amide moiety, an alcohol moiety, a thiol moiety, a cyano moiety, or an ylide moiety.

In some embodiments, the aldehyde-reactive group or first reactive group of the attachment agent is or comprises an amine moiety (e.g., —NHR or —NR₂). The reaction of an amine moiety with an aldehyde moiety of the ribonucleic acid may form an imine or an enamine. As illustrated in FIG. 4A, step 3a, the dialdehydes of the ribonucleic acid are contacted with an attachment agent of Formula III-a or Formula III-b, comprising an amine moiety (e.g., 2-aminoethyl methacrylate or N-(2-aminoethyl)methacrylamide), in the presence of aniline and triethylamine, to form an imine. In FIG. 4B, step 3a, an aldehyde moiety of a ribonucleic acid moiety is similarly reacted with an attachment agent of Formula III-a or Formula III-b, comprising an amine moiety (e.g., 2-aminoethyl (E)-but-2-enoate), in the presence of aniline and triethylamine, to form an imine. In some embodiments, the aldehyde-reactive group or first reactive group is a 2-aminoalkylene moiety. In some embodiments, the aldehyde-reactive group or first reactive group is a 2-aminoethylene moiety.

In some embodiments, the aldehyde-reactive group or first reactive group of the attachment agent is or comprises an amide moiety (e.g., —C(O)—NRR or RC(O)—NR—, wherein R is a general chemical group, such as hydrogen or C₁-C₆alkyl, etc.). The reaction of an amide moiety with an aldehyde moiety of the ribonucleic acid may form an imine. In some embodiments, the attachment agent is acrylamide. In some embodiments, the attachment agent is methyl acrylamide.

In some embodiments, the aldehyde-reactive group or first reactive group of the attachment agent is or comprises an alcohol moiety (e.g., —OH). The reaction of an alcohol moiety with an aldehyde moiety of the ribonucleic acid may form a hemiacetal or acetal.

In some embodiments, the aldehyde-reactive group or first reactive group of the attachment agent is or comprises a thiol moiety (e.g., —SH). The reaction of a thiol moiety with an aldehyde moiety of the ribonucleic acid may form a thioacetal.

In some embodiments, the aldehyde-reactive group or first reactive group of the attachment agent is or comprises a cyano moiety (e.g., —CN). The reaction of a cyano moiety with an aldehyde moiety of the ribonucleic acid may form a cyanohydrin.

In some embodiments, the aldehyde-reactive group or first reactive group of the attachment agent is or comprises an ylide moiety (e.g., —P═CHR). The reaction of an ylide moiety with an aldehyde moiety of the ribonucleic acid may form an alkene.

In some embodiments, the step of contacting the biological sample and attachment agent further comprises contacting the biological sample with one or more reagents or under suitable conditions to facilitate the formation of a covalent bond between the aldehyde(s) of the ribonucleic acid and the aldehyde-reactive group(s) (or first reactive group) of the attachment agent. For example, in some embodiments, the methods provided herein may comprise contacting the attachment agent and the biological sample with aniline and/or triethylamine to facilitate formation of a covalent bond between the aldehyde of the ribonucleic acid and the aldehyde-reactive group of the attachment agent. In some embodiments, the methods comprise contacting the attachment agent and the biological sample with aniline. In some embodiments, the methods comprise contacting the attachment agent and the biological sample with triethylamine. In some embodiments, the methods comprise contacting the attachment agent and the biological sample with aniline and triethylamine. In some embodiments, the methods do not comprise contacting the attachment agent and the biological sample with aniline and/or triethylamine to facilitate formation of a covalent bond between the aldehyde of the ribonucleic acid and the aldehyde-reactive group of the attachment agent. For example, in some embodiments, the formation of a covalent bond between aldehyde of the ribonucleic acid and the aldehyde-reactive group of the attachment agent is facilitated by shifting the equilibrium of the imine formation reaction between the ribonucleic acid and the attachment agent towards the imine. For example, with reference again to FIG. 4A, step 3, and FIG. 4B, step 3, an imine is formed by the reaction of the aldehyde of the ribonucleic acid with the amine moiety of Formula (III-a) or Formula (III-b), comprising an amine moiety (e.g., N-(2-aminoethyl)methacrylamide or 2-aminoethyl methacrylate in FIG. 4A and 2-aminoethyl (E)-but-2-enoate in FIG. 4B). However, as shown in step 4, the imine may equilibrate with the corresponding free aldehyde and amine (as shown in FIG. 4B). Accordingly, in some embodiments, the methods provided herein further comprise shifting equilibrium of the reaction towards the imine and converting the imine to a more stable reaction product, e.g., secondary amine.

For example, with reference again to FIG. 4B, step 4, the imine is further reduced, e.g., by sodium borohydride (NaBH₄) to generate the in some embodiments wherein the attachment agent comprises an amine group, such as in N-(2-aminoethyl)methacrylamide, 2-aminoethyl methacrylate, or 2-aminoethyl (E)-but-2-enoate, or an amide, such as methylacrylamide, and the method may further comprise contacting the biological sample with a reducing agent, optionally wherein the reducing agent is sodium borohydride. In some embodiments, the step of contacting the biological sample and attachment agent further comprises contacting the biological sample with a reducing agent, optionally wherein the reducing agent is sodium borohydride. In some embodiments, the attachment agent is (2-aminoethyl)methacrylamide and the (2-aminoethyl)methacrylamide reacts with at least one aldehyde of the 2′3′-dialdehyde moiety of the ribonucleic acid to form 3′-aminoethyl methacrylamide. In other embodiments, the attachment agent is 2-aminoethyl methacrylate and the 2-aminoethyl methacrylate reacts with at least one aldehyde of the 2′3′-dialdehyde moiety of the ribonucleic acid to form 3′-aminoethyl methacrylate. In yet other embodiments, the attachment agent is 2-aminoethyl (E)-but-2-enoate, and the 2-aminoethyl (E)-but-2-enoate reacts with at least one aldehyde of the 2′3′-dialdehyde moiety of the ribonucleic acid to form 3′-aminoethyl (E)-but-2-enoate.

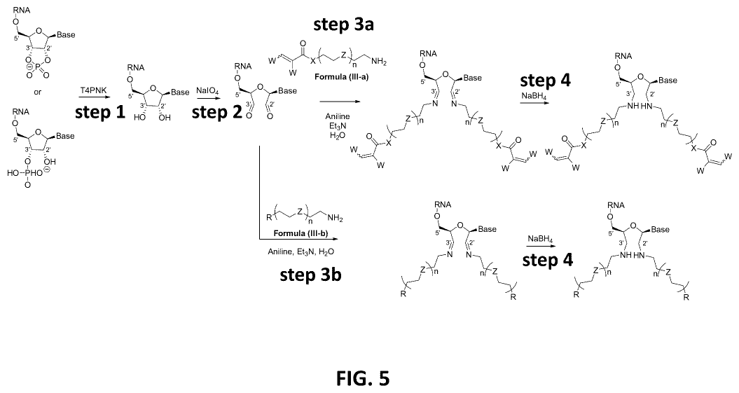

FIG. 5 depicts the combined steps of the reaction schemes shown in FIGS. 4A-4B, beginning with initial treatment of fragmented RNA with a suitable phosphatase to provide 2′,3′-vicinal diols in step 1, conversion of the diols to aldehyde groups in step 2, reaction with attachment agents of Formula III-a in step 3a and Formula III-b in step 3b, and conversion of the resulting imine to a stable amino moiety in step 4.

ii. Attachment Moiety or Second Reactive Group

In some embodiments, the attachment moiety can be any functional group that interacts with an exogenous or endogenous molecule in a biological sample and, in some embodiments, can comprise or be a group capable of reacting with, covalently binding, or non-covalently binding to a complementary reactive group on the exogenous or endogenous molecule.

In some embodiments, the attachment moiety is capable of attaching covalently or non-covalently to an exogenous or endogenous molecule in the biological sample.

In some embodiments, the attachment moiety is capable of attaching covalently to an exogenous or endogenous molecule in the biological sample. In some embodiments, the attachment moiety is capable of attaching covalently to an exogenous molecule in the biological sample. In some embodiments, the attachment moiety is capable of attaching covalently to an endogenous molecule in the biological sample. In some embodiments, the attachment agent is a bifunctional molecule comprising an aldehyde reactive group and an attachment moiety. In such instances in which the attachment agent is a bifunctional molecule the attachment moiety is capable of attaching covalently to an endogenous molecule in the biological sample, it may be referred to herein as a “second reactive group”. In some embodiments, the attachment agent is a bifunctional molecule and the attachment moiety comprises or is a second reactive group capable of reacting with an exogenous or endogenous molecule in the biological sample to form a covalent bond. In some embodiments, the attachment agent is a bifunctional molecule and the attachment moiety comprises or is a second reactive group capable of reacting with an exogenous molecule in the biological sample to form a covalent bond. In some embodiments, the attachment agent is a bifunctional molecule and the attachment moiety comprises or is a second reactive group capable of reacting with an endogenous molecule in the biological sample to form a covalent bond.

In some embodiments, the attachment moiety(ies) or second reactive group comprises or is an electrophilic group that is capable of interacting with a reactive nucleophilic group present on exogenous or endogenous molecule in the biological sample to provide a covalent bond between the attachment moiety and exogenous or endogenous molecule in the biological sample. In some embodiments, the nucleophilic groups on the exogenous or endogenous molecule in the biological sample having that capability include but are not limited to, sulfhydryl, hydroxyl and amino functional groups. In some embodiments, the attachment moiety(ies) or second reactive group comprises or is a maleimide, haloacetamide, or NHS ester.

In some embodiments, the attachment moiety(ies) or second reactive group comprises or is a nucleophilic group that is capable of interacting with a reactive electrophilic group present on exogenous or endogenous molecule in the biological sample to provide a covalent bond between the attachment moiety and exogenous or endogenous molecule in the biological sample. In some embodiments, the attachment moiety(ies) or second reactive group comprises or is a thiol, phenol, amino, hydrazide, hydroxylamine, hydrazine, thiosemicarbazone, hydrazine carboxylate, or arylhydrazide.

In some embodiments, the attachment moiety(ies) or second reactive group comprises or is a click functional group. Suitable click functional groups may include functional groups compatible with a nucleophilic addition reaction, a cyclopropane-tetrazine reaction, a strain-promoted azide-alkyne cycloaddition (SPAAC) reaction, an alkyne hydrothiolation reaction, an alkene hydrothiolation reaction, a strain-promoted alkyne-nitrone cycloaddition (SPANC) reaction, an inverse electron-demand Diels-Alder (IED-DA) reaction, a cyanobenzothiazole condensation reaction, an aldehyde/ketone condensation reaction, and Cu(I)-catalyzed azide-alkyne cycloaddition (CuAAC) reaction. In some embodiments, the attachment moiety(ies) or second reactive group can comprise or be any functional group involved in click reactions. In some embodiments, such click reactions may involve (i) azido and cyclooctynyl; (ii) azido and alkynyl; (iii) tetrazine and dienophile; (iv) thiol and alkynyl; (v) cyano and amino thiol; (vi) nitrone and cyclooctynyl; or (vii) cyclooctynyl and nitrone. It should be recognized that in instances in which the attachment moiety comprises or is a click functional group, the exogenous or endogenous molecule to which it is capable of forming a covalent bond comprises the complementary click functional group to that of the attachment moiety. For example, in some embodiments, the attachment moiety comprises or is an azide moiety and the exogenous or endogenous molecule in the biological sample comprises a complementary alkyne moiety, or vice versa.

In some embodiments, the attachment moiety(ies) or second reactive group comprises or is a group capable of reacting with a matrix-forming agent. In some embodiments, the second reactive group is a reactive group capable of reacting with a matrix-forming agent. As detailed herein, matrix-forming agents may include but are not limited to acrylamide, bis-acrylamide, polyacrylamide and derivatives thereof, poly(ethylene glycol) and derivatives thereof (e.g. PEG-acrylate (PEG-DA), PEG-RGD), gelatin-methacryloyl (GelMA), methacrylated hyaluronic acid (MeHA), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly(hydroxyethyl acrylate), and poly(hydroxyethyl methacrylate), collagen, hyaluronic acid, chitosan, dextran, agarose, gelatin, alginate, protein polymers, methylcellulose, and the like, and combinations thereof. In some embodiments, the attachment moiety(ies) or second reactive group comprises or is an alkenyl, allyl or vinyl moiety, an amide moiety, an alcohol moiety, a polyol moiety, a furan moiety, a maleimide moiety, a norbornene moiety, a thiol moiety, a phenol moiety, a urethane moiety, a cyano moiety, an isocyanate moiety, an isothiocyanate moiety, an ether moiety, a dextran moiety, or an alginate moiety. In some embodiments, the attachment moiety(ies) or second reactive group comprises or is an alkenyl, allyl or vinyl moiety (e.g., —C═C— or HC═C— or HC═C—CH₂—), such as in N-(2-aminoethyl)methacrylamide, 2-aminoethyl methacrylate, 2-aminoethyl (E)-but-2-enoate, 2-aminoethyl methacrylate or methylacrylamide, or norbornene. Such alkenyl, allyl or vinyl moieties may be suitable for reaction with matrix-forming agents.

In some embodiments, the attachment moiety(ies) or second reactive group comprises or is an acrylate moiety, methacrylate moiety, acrylamide moiety, methacrylamide moiety, biotinyl moiety, dextrin moiety, a click moiety, a thiol moiety, norbornenyl moiety, furanyl moiety, alkyl ester moiety, or maleimidyl moiety. In certain embodiments, the attachment moiety(ies) or second reactive group comprises or is a biotinyl moiety, dextrin moiety, a click moiety, a thiol moiety, norbornenyl moiety, furanyl moiety, alkyl ester moiety, or maleimidyl moiety.

In some embodiments, the formation of a covalent or non-covalent bond between the attachment moiety and the exogenous or endogenous molecule in the biological sample is mediated by an external reagent or stimulus. For example, in some embodiments, the formation of a covalent or non-covalent bond between the attachment moiety and the exogenous or endogenous molecule is initiated or induced by an enzyme, a catalyst, chemical reagents (e.g., acid, base, reducing agent, oxidant, etc.), heat, and/or light. In some embodiments, a covalent bond is formed between the attachment moiety and the exogenous or endogenous molecule in the biological sample. In some embodiments, the step of contacting the biological sample and attachment agent further comprises contacting the biological sample with one or more reagents or under suitable conditions to facilitate the formation of a covalent bond between the attachment moiety(ies) or second reactive group of the attachment agent and the exogenous or endogenous molecule in the biological sample. For example, in some embodiments wherein the attachment moiety comprises an alkene or a click functional group, the method may further comprise adding reagents to activate the alkene or click functional group, such as a radical initiator or a copper catalyst, respectively. In other embodiments wherein the attachment moiety(ies) or second reactive group comprises an alkenyl, allyl or vinyl moiety, the method may further comprise exposing the biological sample and attachment agent to (ultraviolet) light or heat to facilitate formation of a covalent bond. In some embodiments wherein the attachment moiety(ies) or second reactive group comprises or is a norbornene moiety, furan moiety, maleimide moiety, or other alkenyl, allyl or vinyl moiety, the method may further comprise exposing the sample to light or heat. In yet other embodiments, the method may further comprise adding an enzyme to facilitate formation of a covalent bond. For example, in some embodiments wherein the attachment moiety(ies) or second reactive group comprises or is a phenol moiety, the method may further comprise adding horseradish peroxidase (HRP).

In some embodiments, the attachment moiety is capable of attaching non-covalently to an exogenous or endogenous molecule in the biological sample. In some embodiments, the attachment moiety is capable of attaching non-covalently to an exogenous molecule in the biological sample. In some embodiments, the attachment moiety is capable of attaching non-covalently to an endogenous molecule in the biological sample. In some embodiments, the attachment moiety comprises or is a group capable of binding to an exogenous or endogenous molecule in the biological sample via non-covalent interaction, such as but not limited to hydrogen bonding, van der Waals interaction, and/or pi-stacking.

In some embodiments, the attachment agent is biotinylated. In some embodiments, the attachment moiety is a biotin moiety or a derivative thereof.

iii. Formulae

In one aspect, provided herein is a compound of Formula (I),

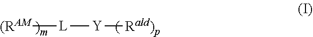

or a salt thereof,
wherein each R^{ald} is independently an aldehyde-reactive group;
Y is —CH₂CH₂— or —O—;
L is a bond or a linker moiety;
each R^{AM} is independently an attachment moiety;
m is an integer from 1 to 4; and
p is an integer from 1 to 4.

In some embodiments, the attachment agent as described herein is a compound of Formula (I). In some embodiments, the attachment agent (e.g., Formula (I)) comprises any of 1, 2, 3, or 4 aldehyde-reactive groups (e.g., R^{a}id). The aldehyde-reactive groups R^{ald} of Formula (I) are each independently selected and as defined in subsection (D) (i) above. In some embodiments, the attachment agent is a bifunctional molecule comprising one aldehyde-reactive group, and the aldehyde-reactive group is a first reactive group.

In some embodiments, R^{ald} comprises or is a nucleophilic group capable of reacting with at least one aldehyde of the 2′,3′-dialdehyde moiety of the ribonucleic acid. In some embodiments, R^{ald} comprises or is an amine moiety, an amide moiety, an alcohol moiety, a thiol moiety, a cyano moiety, an ylide moiety, a hydrazide, a hydroxylamine, a hydrazine, a thiosemicarbazone, a hydrazine carboxylate, or an arylhydrazide, or any combination thereof. In some embodiments, R^{ald} of the attachment agent is or comprises an amine moiety, an amide moiety, an alcohol moiety, a thiol moiety, a cyano moiety, or an ylide moiety.

In some embodiments of Formula (I), p is any of 1, 2, 3, or 4. In some embodiments, the attachment agent (e.g., Formula (I)) comprises more than one aldehyde-reactive groups R^{ald} (e.g., p is any of 2, 3, or 4), wherein the R^{ald} groups can be the same group, selected from the embodiments provided herein. In some embodiments, the attachment agent (e.g., Formula (I)) comprises more than one aldehyde-reactive groups R^{ald} (e.g., p is any of 2, 3, or 4), wherein each R^{ald} is independently selected from the embodiments provided herein, provided the more than one aldehyde reactive groups are chemically compatible and have chemically compatible ribonucleic-binding mechanisms or reactions.

In some embodiments, the attachment agent (e.g., Formula (I)) comprises any of 1, 2, 3, or 4 attachment moieties (e.g., R^{AM}). The attachment moieties R^{AM} of Formula (I) are each independently selected and as defined in subsection (D)(ii) above.

In some embodiments, R^{AM} is capable of reacting with an exogenous or endogenous molecule in the biological sample to form a covalent bond. In some embodiments, R^{AM} is capable of reacting with an exogenous molecule in the biological sample to form a covalent bond. In some embodiments, R^{AM} is capable of reacting with an endogenous molecule in the biological sample to form a covalent bond. In some embodiments, R^{AM} is a maleimide, haloacetamide, or NHS ester. In some embodiments, R^{AM} is a thiol, phenol, amino, hydrazide, hydroxylamine, hydrazine, thiosemicarbazone, hydrazine carboxylate, or arylhydrazide. In some embodiments, R^{AM} is a click functional group.

In some embodiments, R^{AM} is capable of reacting with a matrix-forming agent. In some embodiments, R^{AM} is In some embodiments, R^{AM} is an alkenyl, allyl or vinyl moiety, an amide moiety, an alcohol moiety, a polyol moiety, a furan moiety, a maleimide moiety, a norbornene moiety, a thiol moiety, a phenol moiety, a urethane moiety, a cyano moiety, an isocyanate moiety, an isothiocyanate moiety, an ether moiety, a dextran moiety, or an alginate moiety. In some embodiments, R^{AM} is a biotin moiety or a derivative thereof. In some embodiments, R is an acrylate moiety, methacrylate moiety, acrylamide moiety, methacrylamide moiety, biotinyl moiety, dextrin moiety, a click moiety, a thiol moiety, norbornenyl moiety, furanyl moiety, alkyl ester moiety, or maleimidyl moiety. In certain embodiments, R^{AM} is a biotinyl moiety, dextrin moiety, a click moiety, a thiol moiety, norbornenyl moiety, furanyl moiety, alkyl ester moiety, or maleimidyl moiety.

In some embodiments of Formula (I), m is any of 1, 2, 3, or 4. In some embodiments, the attachment agent (e.g., Formula (I)) comprises more than one attachment moieties R^{AM} (e.g., m is any of 2, 3, or 4), wherein the attachment moieties are the same group, selected from the embodiments provided herein. In some embodiments, the attachment agent (e.g., Formula (I)) comprises more than one attachment moieties R^{AM}(e.g., m is any of 2, 3, or 4), wherein each R^{AM} is independently selected from the embodiments provided herein, provided the more than one reactive groups are chemically compatible and their binding mechanism or reactions to the exogenous or endogenous molecule present in the biological sample are also chemically compatible.

In some embodiments, the compound of formula (I) is a compound of formula (I-a). In some embodiments, the attachment agent is a compound of Formula (I-a), R^{AM}-L-Y—R^{ald} (I-a) or a salt thereof, wherein R^{ald} is an aldehyde-reactive group; Y is —CH₂CH₂— or —O—; L is a bond or a linker moiety; and R^{AM} is independently an attachment moiety. In some embodiments, L is a linker moiety. Examples of linker moieties include but are not limited to a nucleic acid (e.g., a nucleic acid between about 4 and about 200 nucleotides in length, optionally wherein the nucleic acid is between about 4 and about 20 nucleotides in length), a small molecule linker, or a peptide linker. In some embodiments, the linker moiety comprises deoxyribonucleic acid (DNA) and/or locked nucleic acid (LNA). In some embodiments, Z is S (e.g., the linker moiety comprises a disulfide bond. In some embodiments, L is a linker moiety according to formula

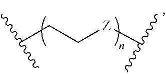

wherein each Z is independently CH₂, O, S, or NH; and n is an integer from 0 to 50. In some embodiments, L is a linker moiety according to formula

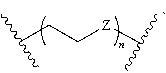

wherein Z is CH₂, O, S, or NH; and n is an integer from 0 to 50.

In some embodiments of Formula (I-a), wherein R^{AM} is capable of attaching covalently to an exogenous or endogenous molecule in the biological sample, R^{ald} is a first reactive group and R^{AM} is a second reactive group.

In some embodiments, the compound of formula (I) is a compound of formula (I-b). In some embodiments, the attachment agent is a compound of Formula (I-b),

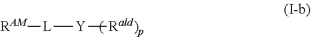

or a salt thereof,
wherein each R^{ald} is independently an aldehyde-reactive group;
Y is —CH₂CH₂— or —O—;
L is a bond or a linker moiety;
R^{AM} is independently an attachment moiety; and
p is an integer from 1 to 4.

In some embodiments, the compound of formula (I) is a compound of formula (I-c). In some embodiments, the attachment agent is a compound of Formula (I-c),

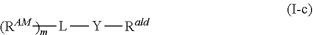

or a salt thereof,
wherein Raid is an aldehyde-reactive group;
Y is —CH₂CH₂— or —O—;
L is a bond or a linker moiety;
each R^{AM} is independently an attachment moiety; and
m is an integer from 1 to 4.

In some embodiments of Formula (I), L is a bond. In some embodiments of Formula (I), L is a linker moiety. In some embodiment, L is an unbranched or branched C₁-C₁₅₀ alkylene, which can be interrupted by 1 to 50 independently selected O, NH, N, S, C₆-C₁₂ arylene, or 5- to 12-membered heteroarylene. In some embodiments, L is an unbranched and uninterrupted C₁-C₁₅₀ alkylene. In some embodiments, L is a branched and uninterrupted C₁-C₁₅₀ alkylene. In some embodiments, L is an unbranched C₁-C₁₅₀ alkylene interrupted by 1 to 50 NH, 0, or S. In some embodiments, L is

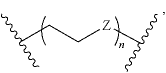

Z is CH₂, O, S; or NH; and n is an integer between 0 and 50. In some embodiments, L is

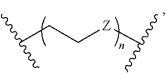

Z is CH₂, O, S; or NH; and n is an integer between 1 and 10. In some embodiment, L is

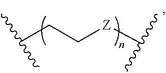

Z is CH₂, O, S; or NH; and n is 6. In some embodiments, L is an unbranched C₁-C₁₅₀ alkylene interrupted by 1 to 50 oxygen. In some embodiments, L comprises a polyethylene glycol portion or is a polyethylene glycol moiety. In some embodiments, L is

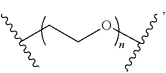

and n is an integer between 0 and 50. In some embodiments, L is

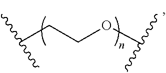

and n is an integer between 1 and 10. In some embodiment, L is

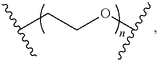

n and n is 6. In some embodiments, L comprises an oligoethylene glycol. In some embodiments, L is an oligoethylene glycol moiety. In some embodiments, L is a branched C₁-C₁₅₀ alkylene interrupted by 1 to 50 oxygen. In some embodiments, L is an unbranched C₁-C₁₅₀ alkylene interrupted by 1 to 50 sulfurs. In some embodiments, L comprises or is

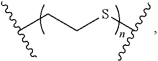

and n is an integer between 0 and 50. In some embodiment, L is

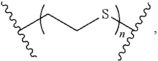

and n is 6. In some embodiments, L is a branched C₁-C₁₅₀ alkylene interrupted by 1 to 50 sulfurs. In some embodiments, L is a branched C₁-C₁₅₀alkylene interrupted by 1 to 50 —NH—. In some embodiments, L is an unbranched C₁-C₁₅₀ alkylene interrupted by 1 to 50 —NH—. In some embodiments, L comprises or is

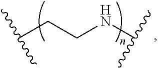

and n is an integer between 0 and 50. In some embodiment, L is

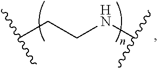

and n is 6. In some embodiments, L is a branched C₁-C₁₅₀ alkylene interrupted by 1 to 50 —NH—, wherein the —NH— is not at a branching point. In some embodiments, L is a branched C₁-C₁₅₀ alkylene interrupted by 1 to 50 —N—, wherein the —N— is at a branching point. In some embodiments, L is an unbranched or branched C₁-C₁₅₀ alkylene interrupted by 1 to 50 independently selected C₆-C₁₂ arylene, for example, any of phenyl or naphthalene. In some embodiments, L is an unbranched or branched C₁-C₁₅₀ alkylene interrupted by 1 to 50 independently selected 5- to 12-membered heteroarylene, for example, any of pyridine, furan, pyrrole, or thiophene.

In some embodiments, Y is —CH₂CH₂—. In some embodiments, Y is O.

In some embodiments, the attachment agent is a compound of Formula (I),

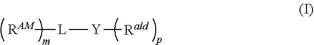

or a salt thereof,
wherein each R^{ald} is independently an aldehyde-reactive group capable of reacting with at least one aldehyde of the 2′,3′-dialdehyde moiety of the ribonucleic acid to form a covalent bond;
Y is —CH₂CH₂— or —O—;
L is a bond or an unbranched or branched C₁-C₁₅₀ alkylene optionally interrupted by 1 to 50 independently selected O, NH, N, S, C₆-C₁₂ arylene, or 5- to 12-membered heteroarylene;
each R^{AM} is independently an attachment moiety capable of attaching covalently or non-covalently to an exogenous or endogenous molecule in the biological sample;
m is an integer from 1 to 4; and
p is an integer from 1 to 4.

In some embodiments, the attachment agent is a compound of Formula (I),

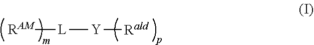

or a salt thereof,
wherein each R^{ald} is independently an aldehyde-reactive group that is a nucleophilic and capable of reacting with at least one aldehyde of the 2′,3′-dialdehyde moiety of the ribonucleic acid to form a covalent bond;
Y is —CH₂CH₂— or —O—;
L is an unbranched or branched C₁-C₁₅₀ alkylene optionally interrupted by 1 to 50 heteroatoms independently selected from the group consisting of 0, S and NH;
each R^{AM} is independently an attachment moiety that is capable of attaching covalently or non-covalently to an exogenous or endogenous molecule in the biological sample;
m is an integer from 1 to 4; and
p is an integer from 1 to 4.

In some embodiments, when Y is —O—, then L is a bond or an unbranched and uninterrupted C₁-C₁₅₀ alkylene.

In some embodiments, the attachment agent is a compound of Formula (I),

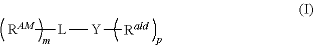

or a salt thereof,
wherein each R^{ald} is independently an aldehyde-reactive group selected from the group consisting of an amine moiety, an amide moiety, an alcohol moiety, a thiol moiety, a cyano moiety, and an ylide moiety;
Y is —CH₂CH₂— or —O—;
L is a bond, an unbranched and uninterrupted C₁-C₁₅₀ alkylene,

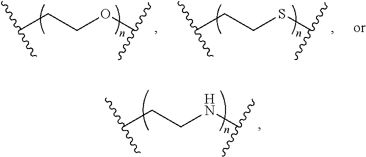

wherein n is an integer from 1 to 50;
each R^{AM} is independently an acrylate moiety, methacrylate moiety, acrylamide moiety, methacrylamide moiety, biotinyl moiety, dextrin moiety, a click moiety, a thiol moiety, norbornenyl moiety, furanyl moiety, alkyl ester moiety, or maleimidyl moiety;
m is an integer from 1 to 4; and
p is an integer from 1 to 4.

In some embodiments, when Y is —O—, then L is a bond or an unbranched and uninterrupted C₁-C₁₅₀ alkylene.

In some embodiments, the attachment agent is a compound of Formula (II-a),

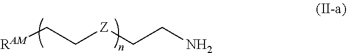

or a salt thereof, wherein
R^{AM} is an attachment moiety as defined herein;
Z is CH₂, O, S; or NH; and
n is an integer from 0 to 50.

In some embodiments, the attachment agent is a compound of Formula (II-b),

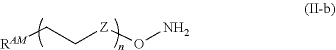

or a salt thereof, wherein
R^{AM} is an attachment moiety as defined herein;
Z is CH₂; and
n is an integer from 0 to 50.

In some embodiments, the attachment agent is a compound of Formula (III-a),

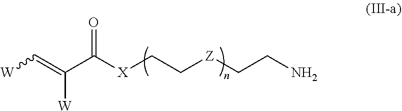

or a salt thereof, wherein
each W is independently H or CH₃;
X is NH or O;
Z is CH₂, O, S; or NH; and
n is an integer from 0 to 50.

In some embodiments, the attachment agent is a compound of Formula (III-2),

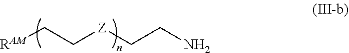

or a salt thereof, wherein
R^{AM} is a biotinyl moiety, dextrin moiety, a click moiety, a thiol moiety, norbornenyl moiety, furanyl moiety, alkyl ester moiety, or maleimidyl moiety;
Z is CH₂, O, S; or NH; and
n is an integer from 0 to 50.

In some embodiments of any of Formulae (I), (I-a), (I-b), (I-c), (II-a), (II-b), (III-a), and (III-b), L is

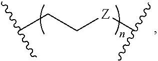

Z is O and n is 6. In some embodiments wherein L is

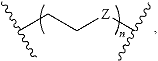

L is a hexaethylene glycol moiety.

In some embodiments, the attachment agent is (2-aminoethyl)methacrylamide, 2-aminoethyl methacrylate, 2-aminoethyl (E)-but-2-enoate, acrylamide, methyl acrylamide, or N-(2-aminoethyl)methacrylamide. In some embodiments, the attachment agent is (2-aminoethyl)methacrylamide. In some embodiments, the attachment agent is 2-aminoethyl methacrylate. In some embodiments, the attachment agent is 2-aminoethyl (E)-but-2-enoate. In some embodiments, the attachment agent is acrylamide. In some embodiments, the attachment agent is methyl acrylamide. In some embodiments, the attachment agent is N-(2-aminoethyl)methacrylamide.

### E. Exogenous or Endogenous Molecule(s)

In some embodiments, the methods of the present disclosure comprise immobilization of a ribonucleic acid modified with an attachment agent having an attachment moiety capable of forming a covalent or non-covalent bond to an exogenous or endogenous molecule.

In some embodiments, the attachment moiety is capable of attaching covalently to an exogenous or endogenous molecule in the biological sample. In some embodiments, the attachment moiety is capable of attaching covalently to an exogenous molecule in the biological sample. In some embodiments, the attachment moiety is capable of attaching covalently to an endogenous molecule in the biological sample. In some embodiments, the attachment moiety is capable of attaching non-covalently to an exogenous or endogenous molecule in the biological sample. In some embodiments, the attachment moiety is capable of attaching non-covalently to an exogenous molecule in the biological sample. In some embodiments, the attachment moiety is capable of attaching non-covalently to an endogenous molecule in the biological sample.

In some embodiments, the exogenous or endogenous molecule in the biological sample to which the attachment moiety is capable of attaching an exogenous molecule. In some embodiments, the exogenous or endogenous molecule in the biological sample to which the attachment moiety is capable of attaching an endogenous molecule.

It should be recognized that the attachment agent (and attachment moiety thereof) and the exogenous or endogenous molecule are selected with respect to one another, such that the exogenous or endogenous molecule has a functional group capable of ligating or bonding to the attachment moiety of the attachment agent. In some embodiments, the exogenous or endogenous molecule comprises a complementary functional group capable of bonding covalently or non-covalently to the attachment moiety of the attachment agent. For example, in some embodiments wherein the attachment agent is N-(2-aminoethyl)methacrylamide, 2-aminoethyl methacrylate, 2-aminoethyl (E)-but-2-enoate, or methacrylamide, the exogenous or endogenous molecule is an acrylamide monomer. In some embodiments wherein the attachment moiety is biotin or the attachment agent is biotinylated, the exogenous or endogenous molecule is streptavidin.

In some embodiments, the exogenous molecule is a matrix-forming agent (e.g., described in Section III), capable of forming a three-dimensional polymerized matrix under suitable reaction conditions. Suitable matrix-forming agents are described herein. For example, in some embodiments, the matrix-forming agent comprises polyacrylamide, cellulose, alginate, polyamide, cross-linked agarose, cross-linked dextran or cross-linked polyethylene glycol. In some embodiments wherein the exogenous molecule is a matrix-forming agent, the method further comprises contacting the biological sample with a matrix-forming agent; and forming a three-dimensional polymerized matrix from the matrix-forming agent, thereby embedding the biological sample in the three-dimensional polymerized matrix and anchoring the ribonucleic acid to the three-dimensional polymerized matrix. In some embodiments wherein the exogenous molecule is a matrix-forming agent and a three-dimensional polymerized matrix has been formed from the matrix-forming agent, the method may further comprise clearing the biological sample embedded in the three-dimensional polymerized matrix. In some embodiments, biological sample is cleared with a detergent, a lipase, and/or a protease.

As detailed herein, in some embodiments, the step of contacting the biological sample and attachment agent further comprises contacting the biological sample with one or more reagents or under suitable conditions to facilitate the formation of a covalent bond between the second reactive group of the attachment agent and the exogenous or endogenous molecule in the biological sample. For example, in some embodiments wherein the attachment moiety comprises an alkenyl, the method may further comprise reagents to activate the alkenyl or click functional group, such as a radical initiator for polymerization.

For example, in some embodiments, the three-dimensional polymerized matrix is formed by subjecting the matrix-forming agent to polymerization. In some embodiments, the polymerization is initiated by adding a polymerization-inducing catalyst, UV light or functional cross-linkers. In some embodiments, the method further comprises staining, permeabilizing, cross-linking, expanding, and/or de-cross-linking the biological sample embedded in the three-dimensional polymerized matrix. In some embodiments, the method further comprises staining, permeabilizing, cross-linking, expanding, and/or de-cross-linking the biological sample embedded in the three-dimensional polymerized matrix after the attachment moiety has been covalently or non-covalently bonded to the matrix-forming agent.

In some embodiments, the exogenous molecule to which the fragmented ribonucleic acid binds through the attachment moiety is a nucleic acid probe. In some embodiments, the nucleic acid probe and the ribonucleic acid hybridize to one another, e.g., they can comprise complementary sequences. In some embodiments, the nucleic acid probe and the ribonucleic acid do not hybridize to one another. In some embodiments, the exogenous molecule to which the fragmented ribonucleic acid binds through the attachment moiety is not a nucleic acid probe. The binding between the exogenous molecule and the fragmented ribonucleic acid through the attachment moiety can be covalent (e.g., covalently linked via a linker) or comprise a non-covalent binding pair (e.g., via a streptavidin/biotin binding pair).

### III. Samples, Analytes, and Detection

### A. Samples and Matrix Embedding

A sample disclosed herein can be or be derived from any biological sample. Methods and compositions disclosed herein may be used for analyzing a biological sample, which may be obtained from a subject using any of a variety of techniques including, but not limited to, biopsy, surgery, and laser capture microscopy (LCM), and generally includes cells and/or other biological material from the subject. In addition to the subjects described above, a biological sample can be obtained from a prokaryote such as a bacterium, an archaeon, a virus, or a viroid. A biological sample can also be obtained from non-mammalian organisms (e.g., a plant, an insect, an arachnid, a nematode, a fungus, or an amphibian). A biological sample can also be obtained from a eukaryote, such as a tissue sample, a patient derived organoid (PDO) or patient derived xenograft (PDX). A biological sample from an organism may comprise one or more other organisms or components therefrom. For example, a mammalian tissue section may comprise a prion, a viroid, a virus, a bacterium, a fungus, or components from other organisms, in addition to mammalian cells and non-cellular tissue components. Subjects from which biological samples can be obtained can be healthy or asymptomatic individuals, individuals that have or are suspected of having a disease (e.g., a patient with a disease such as cancer) or a pre-disposition to a disease, and/or individuals in need of therapy or suspected of needing therapy.

The biological sample can include any number of macromolecules, for example, cellular macromolecules and organelles (e.g., mitochondria and nuclei). The biological sample can be a nucleic acid sample and/or protein sample. The biological sample can be a carbohydrate sample or a lipid sample. The biological sample can be obtained as a tissue sample, such as a tissue section, a cell pellet, a cell block, biopsy, a core biopsy, needle aspirate, or fine needle aspirate. The sample can be a fluid sample, such as a blood sample, urine sample, or saliva sample. The sample can be a skin sample, a colon sample, a cheek swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions. In some embodiments, the biological sample may comprise cells which are deposited on a surface.

Cell-free biological samples can include extracellular polynucleotides. Extracellular polynucleotides can be isolated from a bodily sample, e.g., blood, plasma, serum, urine, saliva, mucosal excretions, sputum, stool, and tears.

Biological samples can be derived from a homogeneous culture or population of the subjects or organisms mentioned herein or alternatively from a collection of several different organisms, for example, in a community or ecosystem.

Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells. Biological samples can also include fetal cells and immune cells.

Biological samples can include analytes (e.g., protein, RNA, and/or DNA) embedded in a 3D matrix. In some embodiments, amplicons (e.g., rolling circle amplification products) derived from or associated with analytes (e.g., protein, RNA, and/or DNA) can be embedded in a 3D matrix. In some embodiments, a 3D matrix may comprise a network of natural molecules and/or synthetic molecules that are chemically and/or enzymatically linked, e.g., by crosslinking. In some embodiments, a 3D matrix may comprise a synthetic polymer. In some embodiments, a 3D matrix comprises a hydrogel.

In some embodiments, a substrate herein can be any support that is insoluble in aqueous liquid and which allows for positioning of biological samples, analytes, features, and/or reagents (e.g., probes) on the support. In some embodiments, a biological sample can be attached to a substrate. Attachment of the biological sample can be irreversible or reversible, depending upon the nature of the sample and subsequent steps in the analytical method. In certain embodiments, the sample can be attached to the substrate reversibly by applying a suitable polymer coating to the substrate and contacting the sample to the polymer coating. The sample can then be detached from the substrate, e.g., using an organic solvent that at least partially dissolves the polymer coating. Hydrogels are examples of polymers that are suitable for this purpose.

In some embodiments, the substrate can be coated or functionalized with one or more substances to facilitate attachment of the sample to the substrate. Suitable substances that can be used to coat or functionalize the substrate include, but are not limited to, lectins, poly-lysine, antibodies, and polysaccharides.

In some embodiments, the biological sample comprises an exogenous or endogenous molecule. In some embodiments, the endogenous molecule comprises a ribonucleic acid. In some embodiments, the biological sample comprises an analyte. In some embodiments, the analyte comprises a ribonucleic acid. In some embodiments, the biological sample comprises a ribonucleic acid.

In some embodiments, the biological sample comprises cells or cellular components. In some embodiments, the biological sample comprises a tissue.

A variety of steps can be performed to prepare or process a biological sample for and/or during an assay. Except where indicated otherwise, the preparative or processing steps described below can generally be combined in any manner and in any order to appropriately prepare or process a particular sample for and/or analysis.

### (i) Tissue Sectioning

A biological sample can be harvested from a subject (e.g., via surgical biopsy, whole subject sectioning) or grown in vitro on a growth substrate or culture dish as a population of cells and prepared for analysis as a tissue slice or tissue section. Grown samples may be sufficiently thin for analysis without further processing steps. Alternatively, grown samples, and samples obtained via biopsy or sectioning, can be prepared as thin tissue sections using a mechanical cutting apparatus such as a vibrating blade microtome. As another alternative, in some embodiments, a thin tissue section can be prepared by applying a touch imprint of a biological sample to a suitable substrate material.

The thickness of the tissue section can be a fraction of (e.g., less than 0.9, 0.8, 0.7, 0.6, 0.5, 0.4, 0.3, 0.2, or 0.1) the maximum cross-sectional dimension of a cell. However, tissue sections having a thickness that is larger than the maximum cross-section cell dimension can also be used. For example, cryostat sections can be used, which can be, e.g., 10-20 μm thick.

More generally, the thickness of a tissue section typically depends on the method used to prepare the section and the physical characteristics of the tissue, and therefore sections having a wide variety of different thicknesses can be prepared and used. For example, the thickness of the tissue section can be at least 0.1, 0.2, 0.3, 0.4, 0.5, 0.7, 1.0, 1.5, 2, 3, 4, 5, 6, 7, 8, 9, 10, 12, 13, 14, 15, 20, 30, 40, or 50 μm. Thicker sections can also be used if desired or convenient, e.g., at least 70, 80, 90, or 100 μm or more. Typically, the thickness of a tissue section is between 1-100 μm, 1-50 μm, 1-30 μm, 1-25 μm, 1-20 μm, 1-15 μm, 1-10 μm, 2-8 μm, 3-7 μm, or 4-6 μm, but as mentioned above, sections with thicknesses larger or smaller than these ranges can also be analysed. In some embodiments, the tissue slice is between about 1-50 μm in thickness.

Multiple sections can also be obtained from a single biological sample. For example, multiple tissue sections can be obtained from a surgical biopsy sample by performing serial sectioning of the biopsy sample using a sectioning blade. Spatial information among the serial sections can be preserved in this manner, and the sections can be analysed successively to obtain three-dimensional information about the biological sample.

### (ii) Freezing

In some embodiments, the biological sample (e.g., a tissue section as described above) can be prepared by deep freezing at a temperature suitable to maintain or preserve the integrity (e.g., the physical characteristics) of the tissue structure. The frozen tissue sample can be sectioned, e.g., thinly sliced, onto a substrate surface using any number of suitable methods. For example, a tissue sample can be prepared using a chilled microtome (e.g., a cryostat) set at a temperature suitable to maintain both the structural integrity of the tissue sample and the chemical properties of the nucleic acids in the sample. Such a temperature can be, e.g., less than −15° C., less than −20° C., or less than −25° C.

In some embodiments, the biological sample is a frozen tissue sample. In other embodiments, the biological sample is a fresh tissue sample.

(iii) Fixation and Postfixation

In some embodiments, the biological sample can be prepared using formalin-fixation and paraffin-embedding (FFPE), which are established methods. In some embodiments, FFPE can be performed prior to embedding the sample in a matrix. In some embodiments, cell suspensions and other non-tissue samples can be prepared using formalin-fixation and paraffin-embedding. Following fixation of the sample and embedding in a paraffin or resin block, the sample can be sectioned as described above. Prior to analysis (e.g., prior to matrix-embedding or introduction of a matrix forming material), the paraffin-embedding material can be removed from the tissue section (e.g., deparaffinization) by incubating the tissue section in an appropriate solvent (e.g., xylene) followed by a rinse (e.g., 99.5% ethanol for 2 minutes, 96% ethanol for 2 minutes, and 70% ethanol for 2 minutes).

As an alternative to formalin fixation described above, a biological sample can be fixed in any of a variety of other fixatives to preserve the biological structure of the sample prior to analysis. For example, a sample can be fixed via immersion in ethanol, methanol, acetone, paraformaldehyde (PFA)-Triton, and combinations thereof.

In some embodiments, acetone fixation is used with fresh frozen samples, which can include, but are not limited to, cortex tissue, mouse olfactory bulb, human brain tumor, human post-mortem brain, and breast cancer samples. When acetone fixation is performed, pre-permeabilization steps (described below) may not be performed. Alternatively, acetone fixation can be performed in conjunction with permeabilization steps.

In some embodiments, the methods provided herein comprise one or more post-fixing (also referred to as postfixation) steps. In some embodiments, one or more post-fixing step is performed after contacting a sample with a polynucleotide disclosed herein, e.g., a probe. In some embodiments, one or more post-fixing step is performed after a hybridization complex comprising the nucleic acid molecule and a target (and optionally, a capture agent) is formed in a sample.

In some embodiments, one or more post-fixing step is performed after contacting a sample with a binding or labelling agent (e.g., an antibody or antigen binding fragment thereof) for a non-nucleic acid analyte such as a protein analyte. The labelling agent can comprise a nucleic acid molecule (e.g., reporter oligonucleotide) comprising a sequence corresponding to the labelling agent and therefore corresponds to (e.g., uniquely identifies) the analyte. In some embodiments, the labelling agent can comprise a reporter oligonucleotide comprising one or more barcode sequences.

A post-fixing step may be performed using any suitable fixation reagent disclosed herein, for example, 3% (w/v) paraformaldehyde in DEPC-PBS.

In some embodiments, the biological sample is fixed. In some embodiments, the biological sample is a FFPE sample.

### (iv) Matrix Embedding

In some embodiments, the biological sample can be embedded in a matrix (e.g., a hydrogel matrix). Any methods and aspects of hydrogel embedding of biological samples are described for example in Chen et al., Science 347(6221):543-548, 2015, the content of which is herein incorporated by reference in its entirety.

In some embodiments, the biological sample comprises a ribonucleic acid. In some embodiments, embedding a biological sample comprising a ribonucleic acid anchors the ribonucleic acid to the matrix. Embedding the sample in this manner typically involves contacting the biological sample with a hydrogel such that the biological sample becomes surrounded by the hydrogel. For example, the sample can be embedded by contacting the sample with a suitable polymer material and activating the polymer material to form a hydrogel.

Examples of matrix-forming agents include acrylamide, bisacrylamide, cellulose, alginate, polyamide, agarose, dextran, or polyethylene glycol. The matrix-forming agents can form a matrix by three-dimensional polymerization and/or crosslinking of the matrix-forming agents using methods specific for the matrix-forming agents and methods, reagents and conditions. In some embodiments, the three-dimensional polymerized matrix is formed by subjecting the matrix-forming agent to polymerization (or to further polymerization, in the case of matrix-forming agents that are polymers such as polyethylene glycol). In some embodiments, the matrix comprises polyacrylamide, cellulose, alginate, polyamide, cross-linked agarose, cross-linked dextran or cross-linked polyethylene glycol.

According to one aspect, a biological sample comprising a ribonucleic acid can be contacted with a matrix-forming agent to form a three-dimensional polymerized matrix thereby embedding the biological sample and anchoring the ribonucleic acid. Forming a three-dimensional polymerized matrix can include adding a polymerization inducing catalyst, UV or functional cross-linker to allow the formation of the three-dimensional polymerized matrix.

In some embodiments, biological samples embedded in the three-dimensional polymerized matrix (e.g., hydrogel) can be cleared using any suitable method. For example, biological samples embedded in the three-dimensional polymerized matrix can be cleared with a detergent, a lipase and/or a protease. In some embodiments, the detergent and lipase remove fatty molecules. In some embodiments, the detergent comprises an ionic detergent or a nonionic detergent. In some embodiments, the detergent comprises a non-ionic surfactant or an anionic surfactant. In some embodiments, the detergent comprises SDS, tergitol, NP-40, saponin, p-(1,1,3,3-tetramethylbutyl)-phenyl ether (also known as Triton X-100™, octyl phenol ethoxylate, polyoxyethylene octyl phenyl ether, 4-octylphenol polyethoxylate, t-octylphenoxypolyethoxyethanol, and octoxynol-9), polysorbate 20 (also known as Tween-20™ polyoxyethylene (20) sorbitan monolaurate, or PEG(20)sorbitan monolaurate), or any combinations thereof. In some embodiments, the lipase comprises a pancreatic, hepatic and/or lysosomal lipase, or any combinations thereof. In some embodiments, the lipase comprises sphingomyelinase or esterase, or a combination thereof. In some embodiments, the protease targets extracellular matrix, fibronectin, collagen and/or elastin. In some embodiments, the protease comprises proteinase K, pepsin, collagenase, trypsin, dispase, thermolysin, or alpha-chymotrypsin, or any combinations thereof. In some embodiments, the protease comprises proteinase K, pepsin, collagenase, trypsin, dispase, thermolysin, or alpha-chymotrypsin, or any combinations thereof. In some embodiments, the protease comprises Liberase™, (Collagenase I, Collagenase II and thermolysin). In other embodiments, electrophoretic tissue clearing methods can be used to remove biological macromolecules from the hydrogel-embedded sample. In some embodiments, a hydrogel-embedded sample is stored before or after clearing of hydrogel, in a medium (e.g., a mounting medium, methylcellulose, or other semi-solid mediums).

According to one aspect, the matrix is sufficiently optically transparent or otherwise has optical properties suitable for deep three-dimensional imaging for high throughput information readout, such as for detection of probe or probe set (i.e., a detectable probe).

According to one aspect, the matrix is porous thereby allowing the introduction of reagents into the matrix at the site of a ribonucleic acid molecule comprising a free 3′ end. Porosity can result from polymerization and/or crosslinking of molecules used to make the matrix material. The diffusion property within the gel matrix is largely a function of the pore size. The molecular sieve size is chosen to allow for rapid diffusion of enzymes, oligonucleotides, formamide and other buffers used for amplification and sequencing (>50-nm). The molecular sieve size is also chosen so that large DNA or RNA amplicons do not readily diffuse within the matrix (<500-nm). The porosity is controlled by changing the cross-linking density, the chain lengths and the percentage of co-polymerized branching monomers. Additional control over the molecular sieve size and density of the matrix is achieved by adding additional cross-linkers such as functionalized polyethylene glycols. In some embodiments, the reagents introduced into the matrix include any of the reagents provided herein. In some embodiments, the reagents comprise a probe or probe set (e.g., detectable probe), amplification reagents (e.g., polymerase), and/or primers.

### (v) Staining and Immunohistochemistry (IHC)

To facilitate visualization, biological samples can be stained using a wide variety of stains and staining techniques. In some embodiments, for example, a sample can be stained using any number of stains and/or immunohistochemical reagents. One or more staining steps may be performed to prepare or process a biological sample for an assay described herein or may be performed during and/or after an assay. In some embodiments, the sample can be contacted with one or more nucleic acid stains, membrane stains (e.g., cellular or nuclear membrane), cytological stains, or combinations thereof. In some examples, the stain may be specific to proteins, phospholipids, DNA (e.g., dsDNA, ssDNA), RNA, an organelle or compartment of the cell. The sample may be contacted with one or more labeled antibodies (e.g., a primary antibody specific for the analyte of interest and a labeled secondary antibody specific for the primary antibody). In some embodiments, cells in the sample can be segmented using one or more images taken of the stained sample.

In some embodiments, the stain is performed using a lipophilic dye. In some examples, the staining is performed with a lipophilic carbocyanine or aminostyryl dye, or analogs thereof (e.g., DiI, DiO, DiR, DiD). Other cell membrane stains may include FM and RH dyes or immunohistochemical reagents specific for cell membrane proteins. In some examples, the stain may include but not limited to, acridine orange, Bismarck brown, carmine, coomassie blue, cresyl violet, DAPI, eosin, ethidium bromide, acid fuchsine, haematoxylin, Hoechst stains, iodine, methyl green, methylene blue, neutral red, Nile blue, Nile red, osmium tetroxide, ruthenium red, propidium iodide, rhodamine (e.g., rhodamine B), or safranine or derivatives thereof. In some embodiments, the sample may be stained with haematoxylin and eosin (H&E).

The sample can be stained using hematoxylin and eosin (H&E) staining techniques, using Papanicolaou staining techniques, Masson's trichrome staining techniques, silver staining techniques, Sudan staining techniques, and/or using Periodic Acid Schiff (PAS) staining techniques. PAS staining is typically performed after formalin or acetone fixation. In some embodiments, the sample can be stained using Romanowsky stain, including Wright's stain, Jenner's stain, Can-Grunwald stain, Leishman stain, and Giemsa stain.

In some embodiments, biological samples can be destained. Any suitable methods of destaining or discoloring a biological sample may be utilized, and generally depend on the nature of the stain(s) applied to the sample. For example, in some embodiments, one or more immunofluorescent stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Methods for multiplexed staining and destaining are described, for example, in Bolognesi et al., J. Histochem. Cytochem. 2017; 65(8): 431-444, Lin et al., Nat Commun. 2015; 6:8390, Pirici et al., J. Histochem. Cytochem. 2009; 57:567-75, and Glass et al., J. Histochem. Cytochem. 2009; 57:899-905, the entire contents of each of which are incorporated herein by reference.

In some embodiments, the embedded biological sample is stained.

### (vi) Isometric Expansion

In some embodiments, a biological sample embedded in a matrix (e.g., a hydrogel) can be isometrically expanded. Isometric expansion methods that can be used include hydration, a preparative step in expansion microscopy, as described in Chen et al., Science 347(6221):543-548, 2015, the content of which is herein incorporated by reference in its entirety.

Isometric expansion can be performed by anchoring one or more components of a biological sample to a gel, followed by gel formation, proteolysis, and swelling. In some embodiments, analytes in the sample, products of the analytes, and/or probes associated with analytes in the sample can be anchored to the matrix (e.g., hydrogel). Isometric expansion of the biological sample can occur prior to immobilization of the biological sample on a substrate, or after the biological sample is immobilized to a substrate. In some embodiments, the isometrically expanded biological sample can be removed from the substrate prior to contacting the substrate with probes disclosed herein.

In general, the steps used to perform isometric expansion of the biological sample can depend on the characteristics of the sample (e.g., thickness of tissue section, fixation, cross-linking), and/or the analyte of interest (e.g., different conditions to anchor RNA, DNA, and protein to a gel).

In some embodiments, proteins in the biological sample are anchored to a swellable gel such as a polyelectrolyte gel. An antibody can be directed to the protein before, after, or in conjunction with being anchored to the swellable gel. DNA and/or RNA in a biological sample can also be anchored to the swellable gel via a suitable linker. Examples of such linkers include, but are not limited to, 6-((Acryloyl)amino) hexanoic acid (Acryloyl-X SE) (available from ThermoFisher, Waltham, MA), Label-IT Amine (available from MirusBio, Madison, WI) and Label X (described for example in Chen et al., Nat. Methods 13:679-684, 2016, the content of which is herein incorporated by reference in its entirety).

Isometric expansion of the sample can increase the spatial resolution of the subsequent analysis of the sample. The increased resolution in spatial profiling can be determined by comparison of an isometrically expanded sample with a sample that has not been isometrically expanded.

In some embodiments, a biological sample is isometrically expanded to a size at least 2×, 2.1×, 2.2×, 2.3×, 2.4×, 2.5×, 2.6×, 2.7×, 2.8×, 2.9×, 3×, 3.1×, 3.2×, 3.3×, 3.4×, 3.5×, 3.6×, 3.7×, 3.8×, 3.9×, 4×, 4.1×, 4.2×, 4.3×, 4.4×, 4.5×, 4.6×, 4.7×, 4.8×, or 4.9× its non-expanded size. In some embodiments, the sample is isometrically expanded to at least 2× and less than 20× of its non-expanded size.

In some embodiments, the embedded biological sample is isometrically expanded.

(vii) Crosslinking and De-crosslinking

In some embodiments, the biological sample is reversibly cross-linked prior to or during an in situ assay round.

In some embodiments, the biological sample is immobilized in a hydrogel via cross-linking of the polymer material that forms the hydrogel. Cross-linking can be performed chemically and/or photochemically, or alternatively by any other hydrogel-formation method. A hydrogel may include a macromolecular polymer gel including a network. Within the network, some polymer chains can optionally be cross-linked, although cross-linking does not always occur.

In some embodiments, a hydrogel can include hydrogel subunits, such as, but not limited to, acrylamide, bis-acrylamide, polyacrylamide and derivatives thereof, poly(ethylene glycol) and derivatives thereof (e.g. PEG-acrylate (PEG-DA), PEG-RGD), gelatin-methacryloyl (GelMA), methacrylated hyaluronic acid (MeHA), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly(hydroxyethyl acrylate), and poly(hydroxyethyl methacrylate), collagen, hyaluronic acid, chitosan, dextran, agarose, gelatin, alginate, protein polymers, methylcellulose, and the like, and combinations thereof.

In some embodiments, a hydrogel includes a hybrid material, e.g., the hydrogel material includes elements of both synthetic and natural polymers. Examples of suitable hydrogels are described, for example, in U.S. Pat. Nos. 6,391,937, 9,512,422, and 9,889,422, and in U.S. Patent Application Publication Nos. 2017/0253918, 2018/0052081, 2010/0055733, and 2020/0071751 the entire contents of each of which are incorporated herein by reference.

In some embodiments, the hydrogel can form the substrate. In some embodiments, the substrate includes a hydrogel and one or more second materials. In some embodiments, the hydrogel is placed on top of one or more second materials. For example, the hydrogel can be pre-formed and then placed on top of, underneath, or in any other configuration with one or more second materials. In some embodiments, hydrogel formation occurs after contacting one or more second materials during formation of the substrate. Hydrogel formation can also occur within a structure (e.g., wells, ridges, projections, and/or markings) located on a substrate.

In some embodiments, hydrogel formation on a substrate occurs before, contemporaneously with, or after capture agents, labelling agents, and/or probes are provided to the sample. For example, hydrogel formation can be performed on the substrate already containing the probes.

In some embodiments, hydrogel formation occurs within a biological sample. In some embodiments, a biological sample (e.g., tissue section) is embedded in a hydrogel. In some embodiments, hydrogel subunits are infused into the biological sample, and polymerization of the hydrogel is initiated by an external or internal stimulus.

In embodiments in which a hydrogel is formed within a biological sample, functionalization chemistry can be used. In some embodiments, functionalization chemistry includes hydrogel-tissue chemistry (HTC). Any hydrogel-tissue backbone (e.g., synthetic or native) suitable for HTC can be used for anchoring biological macromolecules and modulating functionalization. Non-limiting examples of methods using HTC backbone variants include CLARITY, PACT, ExM, SWITCH and ePACT. In some embodiments, hydrogel formation within a biological sample is permanent. For example, biological macromolecules can permanently adhere to the hydrogel allowing multiple rounds of interrogation. In some embodiments, hydrogel formation within a biological sample is reversible.

In some embodiments, additional reagents are added to the hydrogel subunits before, contemporaneously with, and/or after polymerization. For example, additional reagents can include but are not limited to oligonucleotides (e.g., probes), endonucleases to fragment DNA, fragmentation buffer for DNA, DNA polymerase enzymes (e.g., phi29 DNA polymerases, Bst DNA polymerases, and Bsu DNA polymerase, large fragment), and dNTPs used to extend the nucleic acid molecule and generate an elongated product in the sample. Other enzymes can be used, including without limitation, RNA polymerase, transposase, ligase, proteinase K, and DNAse. Additional reagents can also include reverse transcriptase enzymes, including enzymes with terminal transferase activity, primers, and switch oligonucleotides. In some embodiments, optical labels are added to the hydrogel subunits before, contemporaneously with, and/or after polymerization.

In some embodiments, HTC reagents are added to the hydrogel before, contemporaneously with, and/or after polymerization. In some embodiments, a cell labelling agent is added to the hydrogel before, contemporaneously with, and/or after polymerization. In some embodiments, a cell-penetrating agent is added to the hydrogel before, contemporaneously with, and/or after polymerization.

Hydrogels embedded within biological samples can be cleared using any suitable method. For example, electrophoretic tissue clearing methods can be used to remove biological macromolecules from the hydrogel-embedded sample. In some embodiments, a hydrogel-embedded sample is stored before or after clearing of hydrogel, in a medium (e.g., a mounting medium, methylcellulose, or other semi-solid mediums).

In some embodiments, a method disclosed herein comprises de-crosslinking the reversibly cross-linked biological sample. The de-crosslinking does not need to be complete. In some embodiments, only a portion of crosslinked molecules in the reversibly cross-linked biological sample are de-crosslinked and allowed to migrate.

In some embodiments, the embedded biological sample is cross-linked. In some embodiments, the embedded biological sample is cross-linked and de-cross-linked.

(viii) Tissue Permeabilization and Treatment

In some embodiments, a biological sample can be permeabilized to facilitate transfer of analytes out of the sample, and/or to facilitate transfer of species (such as a probe or probe set, labelling agents, and/or capture agents) into the sample. If a sample is not permeabilized sufficiently, the amount of analyte captured from the sample may be too low to enable adequate analysis. Conversely, if the tissue sample is too permeable, the relative spatial relationship of the analytes within the tissue sample can be lost. Hence, a balance between permeabilizing the tissue sample enough to obtain good signal intensity while still maintaining the spatial resolution of the analyte distribution in the sample is desirable.

In general, a biological sample can be permeabilized by exposing the sample to one or more permeabilizing agents. Suitable agents for this purpose include, but are not limited to, organic solvents (e.g., acetone, ethanol, and methanol), cross-linking agents (e.g., paraformaldehyde), detergents such as SDS, tergitol, NP-40, saponin, p-(1,1,3,3-tetramethylbutyl)-phenyl ether (also known as Triton X-100™, octyl phenol ethoxylate, polyoxyethylene octyl phenyl ether, 4-octylphenol polyethoxylate, t-octylphenoxypolyethoxyethanol, octoxynol-9), polysorbate 20 (also known as Tween-20™, also known as polyoxyethylene (20) sorbitan monolaurate, or PEG(20)sorbitan monolaurate), and enzymes (e.g., proteases such as proteinase K, pepsin, collagenase, trypsin, Liberase™, dispase, thermolysin, or alpha-chymotrypsin). In some embodiments, the biological sample can be incubated with a cellular permeabilizing agent to facilitate permeabilization of the sample. Additional methods for sample permeabilization are described, for example, in Jamur et al., Method Mol. Biol. 588:63-66, 2010, the content of which is herein incorporated by reference in its entirety. Any suitable method for sample permeabilization can generally be used in connection with the samples described herein.

In some embodiments, the biological sample can be permeabilized by adding one or more lysis reagents to the sample. Examples of suitable lysis agents include, but are not limited to, bioactive reagents such as lysis enzymes that are used for lysis of different cell types, e.g., gram positive or negative bacteria, plants, yeast, mammalian, such as lysozymes, achromopeptidase, lysostaphin, labiase, kitalase, lyticase, and a variety of other commercially available lysis enzymes.

Other lysis agents can additionally or alternatively be added to the biological sample to facilitate permeabilization. For example, surfactant-based lysis solutions can be used to lyse sample cells. Lysis solutions can include ionic surfactants such as, for example, sarcosyl and sodium dodecyl sulfate (SDS). More generally, chemical lysis agents can include, without limitation, organic solvents, chelating agents, detergents, surfactants, and chaotropic agents.

In some embodiments, the biological sample can be permeabilized by non-chemical permeabilization methods. For example, non-chemical permeabilization methods that can be used include, but are not limited to, physical lysis techniques such as electroporation, mechanical permeabilization methods (e.g., bead beating using a homogenizer and grinding balls to mechanically disrupt sample tissue structures), acoustic permeabilization (e.g., sonication), and thermal lysis techniques such as heating to induce thermal permeabilization of the sample.

Additional reagents can be added to a biological sample to perform various functions prior to analysis of the sample. In some embodiments, RNase inactivating agents or inhibitors such as proteinase K, and/or chelating agents such as EDTA, can be added to the sample.

In some embodiments, the embedded biological sample is permeabilized.

### (ix) Selective Enrichment of RNA Species

In some embodiments, where RNA is the analyte, one or more RNA analyte species of interest can be selectively enriched. For example, one or more species of RNA of interest can be selected by addition of one or more oligonucleotides to the sample. In some embodiments, the additional oligonucleotide is a sequence used for priming a reaction by an enzyme (e.g., a polymerase). For example, one or more primer sequences with sequence complementarity to one or more RNAs of interest can be used to amplify the one or more RNAs of interest, thereby selectively enriching these RNAs.

In some embodiments, an oligonucleotide with sequence complementarity to the complementary strand of captured RNA (e.g., cDNA) can bind to the cDNA. For example, biotinylated oligonucleotides with sequence complementary to one or more cDNA of interest binds to the cDNA and can be selected using biotinylation-streptavidin affinity using any of a variety of methods (e.g., streptavidin beads).

A biological sample may comprise one or a plurality of analytes of interest. Methods for performing multiplexed assays to analyze two or more different analytes in a single biological sample are provided.

### B. Analytes

The methods disclosed herein can be used to detect and analyze a wide variety of different analytes (e.g., including RNA as described in Section II and in combination with other analytes). In some aspects, an analyte can include any biological substance, structure, moiety, or component to be analyzed. In some aspects, a target disclosed herein may similarly include any analyte of interest. In some examples, a target or analyte can be directly or indirectly detected.

Analytes can be derived from a specific type of cell and/or a specific sub-cellular region. For example, analytes can be derived from cytosol, from cell nuclei, from mitochondria, from microsomes, and more generally, from any other compartment, organelle, or portion of a cell. Permeabilizing agents that specifically target certain cell compartments and organelles can be used to selectively release analytes from cells for analysis, and/or allow access of one or more reagents (e.g., probes for analyte detection) to the analytes in the cell or cell compartment or organelle.

The analyte may include any biomolecule or chemical compound, including a macromolecule such as a protein or peptide, a lipid or a nucleic acid molecule, or a small molecule, including organic or inorganic molecules. The analyte may be a cell or a microorganism, including a virus, or a fragment or product thereof. An analyte can be any substance or entity for which a specific binding partner (e.g., an affinity binding partner) can be developed. Such a specific binding partner may be a nucleic acid probe (for a nucleic acid analyte).

Analytes of particular interest may include nucleic acid molecules, such as RNA (e.g. mRNA, microRNA, rRNA, snRNA, viral RNA, etc.), and synthetic and/or modified nucleic acid molecules, (e.g. including nucleic acid domains comprising or consisting of synthetic or modified nucleotides such as LNA, PNA, morpholino, etc.), proteinaceous molecules such as peptides, polypeptides, proteins or prions or any molecule which includes a protein or polypeptide component, etc., or fragments thereof, or a lipid or carbohydrate molecule, or any molecule which comprise a lipid or carbohydrate component. The analyte may be a single molecule or a complex that contains two or more molecular subunits, e.g., including but not limited to protein-RNA complexes, which may or may not be covalently bound to one another, and which may be the same or different. Thus, in addition to cells or microorganisms, such a complex analyte may also be a protein complex or protein interaction. Such a complex or interaction may thus be a homo- or hetero-multimer. Aggregates of molecules, e.g., proteins may also be target analytes, for example aggregates of the same protein or different proteins. The analyte may also be a complex between proteins or peptides and nucleic acid molecules such as RNA, e.g., interactions between proteins and nucleic acids, e.g., regulatory factors, such as transcription factors, and RNA.

### (i) Endogenous Analytes

In some embodiments, an analyte herein is endogenous to a biological sample and can include nucleic acid analytes and non-nucleic acid analytes. Methods and compositions disclosed herein can be used to analyze nucleic acid analytes (e.g., by immobilizing or tethering any fragmented ribonucleic acids to an endogenous molecule in the biological sample or an exogenous molecule delivered to the biological sample as described in Section II and using a nucleic acid probe or probe set that directly or indirectly hybridizes to the immobilized or tethered nucleic acid analyte).

Examples of nucleic acid analytes include RNA analytes such as various types of coding and non-coding RNA. Examples of the different types of RNA analytes include messenger RNA (mRNA), including a nascent RNA, a pre-mRNA, a primary-transcript RNA, and a processed RNA, such as a capped mRNA (e.g., with a 5′ 7-methyl guanosine cap), a polyadenylated mRNA (poly-A tail at the 3′ end), and a spliced mRNA in which one or more introns have been removed. Also included in the analytes disclosed herein are non-capped mRNA, a non-polyadenylated mRNA, and a non-spliced mRNA. The RNA analyte can be a transcript of another nucleic acid molecule (e.g., DNA or RNA such as viral RNA) present in a tissue sample. Examples of a non-coding RNAs (ncRNA) that is not translated into a protein include transfer RNAs (tRNAs) and ribosomal RNAs (rRNAs), as well as small non-coding RNAs such as microRNA (miRNA), small interfering RNA (siRNA), Piwi-interacting RNA (piRNA), small nucleolar RNA (snoRNA), small nuclear RNA (snRNA), extracellular RNA (exRNA), small Cajal body-specific RNAs (scaRNAs), and the long ncRNAs such as Xist and HOTAIR. The RNA can be small (e.g., less than 200 nucleic acid bases in length) or large (e.g., RNA greater than 200 nucleic acid bases in length). Examples of small RNAs include 5.8S ribosomal RNA (rRNA), 5S rRNA, tRNA, miRNA, siRNA, snoRNAs, piRNA, tRNA-derived small RNA (tsRNA), and small rDNA-derived RNA (srRNA). The RNA can be double-stranded RNA or single-stranded RNA. The RNA can be circular RNA. The RNA can be a bacterial rRNA (e.g., 16s rRNA or 23s rRNA). In some embodiments described herein, an analyte may be a fragmented RNA.

In certain embodiments, an analyte can be extracted from a live cell. Processing conditions can be adjusted to ensure that a biological sample remains live during analysis, and analytes are extracted from (or released from) live cells of the sample. Live cell-derived analytes can be obtained only once from the sample or can be obtained at intervals from a sample that continues to remain in viable condition.

Methods and compositions disclosed herein can be used to analyze any number of analytes. For example, the number of analytes that are analyzed can be at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 40, at least about 50, at least about 100, at least about 1,000, at least about 10,000, at least about 100,000 or more different analytes present in a region of the sample or within an individual feature of the substrate.

In any embodiment described herein, the analyte comprises a target sequence. In some embodiments, the target sequence may be endogenous to the sample, generated in the sample, added to the sample, or associated with an analyte in the sample. In some embodiments, the target sequence is a single-stranded target sequence (e.g., a sequence in a rolling circle amplification product). In some embodiments, the analytes comprise one or more single-stranded target sequences. In one aspect, a first single-stranded target sequence is not identical to a second single-stranded target sequence. In another aspect, a first single-stranded target sequence is identical to one or more second single-stranded target sequence. In some embodiments, the one or more second single-stranded target sequence is comprised in the same analyte (e.g., nucleic acid) as the first single-stranded target sequence. Alternatively, the one or more second single-stranded target sequence is comprised in a different analyte (e.g., nucleic acid) from the first single-stranded target sequence.

### (ii) Labelling Agents

In some embodiments, provided herein are methods and compositions for analyzing endogenous analytes (e.g., RNA) in a sample and other analytes using one or more labelling agents. In some aspects, the methods for immobilizing or tethering fragmented ribonucleic acids to an endogenous molecule in the biological sample or an exogenous molecule delivered to the biological sample as described in Section II is compatible with protein analysis (e.g., using a labelling agent). In some embodiments, an analyte labelling agent may include an agent that interacts with an analyte (e.g., an endogenous analyte in a sample). In some embodiments, the labelling agents can comprise a reporter oligonucleotide that is indicative of the analyte or portion thereof interacting with the labelling agent. For example, the reporter oligonucleotide may comprise a barcode sequence that permits identification of the labelling agent. In some cases, the sample contacted by the labelling agent can be further contacted with a probe (e.g., a single-stranded probe sequence), that hybridizes to a reporter oligonucleotide of the labelling agent, in order to identify the analyte associated with the labelling agent. In some embodiments, the analyte labelling agent comprises an analyte binding moiety and a labelling agent barcode domain comprising one or more barcode sequences, e.g., a barcode sequence that corresponds to the analyte binding moiety and/or the analyte. An analyte binding moiety barcode includes to a barcode that is associated with or otherwise identifies the analyte binding moiety. In some embodiments, by identifying an analyte binding moiety by identifying its associated analyte binding moiety barcode, the analyte to which the analyte binding moiety binds can also be identified. An analyte binding moiety barcode can be a nucleic acid sequence of a given length and/or sequence that is associated with the analyte binding moiety. An analyte binding moiety barcode can generally include any of the variety of aspects of barcodes described herein.

In some embodiments, the method comprises one or more post-fixing (also referred to as post-fixation) steps after contacting the sample with one or more labelling agents.

In some embodiments, an analyte binding moiety may include any molecule or moiety capable of binding to an analyte (e.g., a biological analyte, e.g., a macromolecular constituent). A labelling agent may include, but is not limited to, a protein, a peptide, an antibody (or an epitope binding fragment thereof), a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a darpin, and a protein scaffold, or any combination thereof. The labelling agents can include (e.g., are attached to) a reporter oligonucleotide that is indicative of the cell surface feature to which the binding group binds. For example, the reporter oligonucleotide may comprise a barcode sequence that permits identification of the labelling agent. For example, a labelling agent that is specific to one type of cell feature (e.g., a first cell surface feature) may have coupled thereto a first reporter oligonucleotide, while a labelling agent that is specific to a different cell feature (e.g., a second cell surface feature) may have a different reporter oligonucleotide coupled thereto. For a description of exemplary labelling agents, reporter oligonucleotides, and methods of use, see, e.g., U.S. Pat. No. 10,550,429; U.S. Pat. Pub. 20190177800; and U.S. Pat. Pub. 20190367969, which are each incorporated by reference herein in their entirety.

In some embodiments, an analyte binding moiety includes one or more antibodies or antigen binding fragments thereof. The antibodies or antigen binding fragments including the analyte binding moiety can specifically bind to a target analyte. In some embodiments, the analyte is a protein (e.g., a protein on a surface of the biological sample (e.g., a cell) or an intracellular protein). In some embodiments, a plurality of analyte labelling agents comprising a plurality of analyte binding moieties bind a plurality of analytes present in a biological sample. In some embodiments, the plurality of analytes includes a single species of analyte (e.g., a single species of polypeptide). In some embodiments in which the plurality of analytes includes a single species of analyte, the analyte binding moieties of the plurality of analyte labelling agents are the same. In some embodiments in which the plurality of analytes includes a single species of analyte, the analyte binding moieties of the plurality of analyte labelling agents are the different (e.g., members of the plurality of analyte labelling agents can have two or more species of analyte binding moieties, wherein each of the two or more species of analyte binding moieties binds a single species of analyte, e.g., at different binding sites). In some embodiments, the plurality of analytes includes multiple different species of analyte (e.g., multiple different species of polypeptides).

In other instances, e.g., to facilitate sample multiplexing, a labelling agent that is specific to a particular cell feature may have a first plurality of the labelling agent (e.g., an antibody or lipophilic moiety) coupled to a first reporter oligonucleotide and a second plurality of the labelling agent coupled to a second reporter oligonucleotide.

In some aspects, these reporter oligonucleotides may comprise nucleic acid barcode sequences that permit identification of the labelling agent which the reporter oligonucleotide is coupled to. The selection of oligonucleotides as the reporter may provide advantages of being able to generate significant diversity in terms of sequence, while also being readily attachable to most biomolecules, e.g., antibodies, etc., as well as being readily detected, e.g., using sequencing or array technologies.

Attachment (coupling) of the reporter oligonucleotides to the labelling agents may be achieved through any of a variety of direct or indirect, covalent or non-covalent associations or attachments. In some embodiments, the oligonucleotide attached to a labelling agent comprises a sequence that can serve as a primer and can be used as a reporter (e.g., a barcode). In some embodiments, the oligonucleotide attached to a labelling agent comprises both a reporter sequence (e.g., a barcode) and a sequence serving as a primer. For example, oligonucleotides may be covalently attached to a portion of a labelling agent (such a protein, e.g., an antibody or antibody fragment) using chemical conjugation techniques (e.g., Lightning-Link® antibody labelling kits available from Innova Biosciences), as well as other non-covalent attachment mechanisms, e.g., using biotinylated antibodies and oligonucleotides (or beads that include one or more biotinylated linker, coupled to oligonucleotides) with an avidin or streptavidin linker. Antibody and oligonucleotide biotinylation techniques are available. See, e.g., Fang, et al., “Fluoride-Cleavable Biotinylation Phosphoramidite for 5′-end-Labelling and Affinity Purification of Synthetic Oligonucleotides,” Nucleic Acids Res. Jan. 15, 2003; 31(2):708-715, the content of which is herein incorporated by reference in its entirety. Likewise, protein and peptide biotinylation techniques have been developed and are readily available. See, e.g., U.S. Pat. No. 6,265,552, which is entirely incorporated herein by reference for all purposes. Furthermore, click reaction chemistry may be used to couple reporter oligonucleotides to labelling agents. Commercially available kits, such as those from Thunderlink and Abcam, and techniques common in the art may be used to couple reporter oligonucleotides to labelling agents as appropriate. In another example, a labelling agent is indirectly (e.g., via hybridization) coupled to a reporter oligonucleotide comprising a barcode sequence that identifies the labelling agent. For instance, the labelling agent may be directly coupled (e.g., covalently bound) to a hybridization oligonucleotide that comprises a sequence that hybridizes with a sequence of the reporter oligonucleotide. Hybridization of the hybridization oligonucleotide to the reporter oligonucleotide couples the labelling agent to the reporter oligonucleotide. In some embodiments, the reporter oligonucleotides are releasable from the labelling agent, such as upon application of a stimulus. For example, the reporter oligonucleotide or primer may be attached to the labeling agent through a labile bond (e.g., chemically labile, photolabile, thermally labile, etc.) as generally described for releasing molecules from supports elsewhere herein. In some instances, the reporter oligonucleotides described herein may include one or more functional sequences that can be used in subsequent processing, such as an adapter sequence or a unique molecular identifier (UMI) sequence.

In some cases, the labelling agent can comprise a reporter oligonucleotide and a label. A label can be fluorophore, a radioisotope, a molecule capable of a colorimetric reaction, a magnetic particle, or any other suitable molecule or compound capable of detection. The label can be conjugated to a labelling agent (or reporter oligonucleotide) either directly or indirectly (e.g., the label can be conjugated to a molecule that can bind to the labelling agent or reporter oligonucleotide). In some cases, a label is conjugated to a first oligonucleotide that is complementary (e.g., hybridizes) to a sequence of the reporter oligonucleotide.

### C. Detection and Analysis

In some aspects, the provided methods involve analyzing the tethered or immobilized RNA as described in Section II and optionally other analytes, e.g., by detecting or determining, one or more sequences present in probes or probe sets or products thereof (e.g., rolling circle amplification products (RCPs) thereof). In some embodiments, the detecting is performed at one or more locations in a biological sample. In some embodiments, the locations are the locations of tethered or immobilized RNA transcripts in the biological sample. In some embodiments, the locations are the locations at which probes or probe sets hybridize to the RNA transcripts in the biological sample and are optionally ligated and amplified by rolling circle amplification. In some embodiments, the number of detected RCPs, size of RCPs and/or signal to background ratio is improved in tethered samples compared to samples where RNA is not tethered. In some embodiments, the number of detected RCPs, size of RCPs and/or signal to background is improved in tethered samples treated with a clearing agent (e.g., a detergent, a lipase, and/or a protease) compared to sample where RNA is not tethered.

In some embodiments, the method provided herein comprises contacting the biological sample with a probe or probe set that binds directly or indirectly to the ribonucleic acid. In some embodiments, the probe or probe set is a detectable probe. In some embodiments, the probe or probe set comprises a barcode sequence. In some embodiments, the probe or probe set comprises an intermediate probe and a detection oligonucleotide. In some embodiments, the detecting comprises a plurality of repeated cycles of hybridization and removal of probes (e.g., detectably labeled probes, or intermediate probes that bind to detectably labeled probes) to the primary probe or probe set hybridized to the target nucleic acid, or to a rolling circle amplification product generated from the probe or probe set hybridized to the target nucleic acid. In some embodiments, a single probe or probe set is hybridized to each target nucleic acid. In some aspects, number of detected RCPs, size of RCPs and/or signal to background ratio is improved in tethered samples (e.g., as described in Section II) compared to samples where RNA is not tethered and only one probe or probe set is needed to bind each target nucleic acid (e.g., RNA) to generate sufficient signal for detection.

In some embodiments, the method comprises contacting the biological sample with a circular or circularizable probe, wherein the circular or circularizable probe binds the fragmented ribonucleic acid and generates a rolling circle amplification (RCA) product.

In some embodiments, the method comprises imaging the biological sample to detect the RCA product. In some embodiments, imaging comprises detecting a signal associated the probe, optionally with a fluorescently labeled probe that directly or indirectly binds to the rolling circle amplification product.

### (a) Hybridization, Ligation, and Amplification

In some embodiments, the method comprises contacting the biological sample with a probe or probe set that hybridizes directly or indirectly to the ribonucleic acid. For purposes of hybridization, two nucleic acid sequences are “substantially complementary” if at least 60% (e.g., at least 70%, at least 80%, or at least 90%) of their individual bases are complementary to one another. Various probes and probe sets can be hybridized to an endogenous analyte (e.g., ribonucleic acid) and/or a labelling agent.

In some embodiments, the method comprises generating a ligation product with a probe or probe set that hybridizes directly or indirectly to the ribonucleic acid.

In some embodiments, provided herein is a probe or probe set capable of DNA-templated ligation, such as from a cDNA molecule. See, e.g., U.S. Pat. No. 8,551,710, which is hereby incorporated by reference in its entirety. In some embodiments, provided herein is a probe or probe set capable of RNA-templated ligation. See, e.g., U.S. Pat. Pub. 2020/0224244 which is hereby incorporated by reference in its entirety. In some embodiments, the probe set is a SNAIL probe set. See, e.g., U.S. Pat. Pub. 20190055594, which is hereby incorporated by reference in its entirety. In some embodiments, provided herein is a multiplexed proximity ligation assay. See, e.g., U.S. Pat. Pub. 20140194311 which is hereby incorporated by reference in its entirety. In some embodiments, provided herein is a probe or probe set capable of proximity ligation, for instance a proximity ligation assay for RNA (e.g., PLAYR) probe set. See, e.g., U.S. Pat. Pub. 20160108458, which is hereby incorporated by reference in its entirety. In some embodiments, a circular probe can be indirectly hybridized to the target nucleic acid. In some embodiments, the circular construct is formed from a probe set capable of proximity ligation, for instance a proximity ligation in situ hybridization (PLISH) probe set. See, e.g., U.S. Pat. Pub. 2020/0224243 which is hereby incorporated by reference in its entirety.

In some embodiments, the ligation involves chemical ligation. In some embodiments, the ligation involves template dependent ligation. In some embodiments, the ligation involves template independent ligation. In some embodiments, the ligation involves enzymatic ligation.

In some embodiments, the enzymatic ligation involves use of a ligase. In some aspects, the ligase used herein comprises an enzyme that is commonly used to join polynucleotides together or to join the ends of a single polynucleotide. An RNA ligase, a DNA ligase, or another variety of ligase can be used to ligate two nucleotide sequences together. Ligases comprise ATP-dependent double-strand polynucleotide ligases, NAD-i-dependent double-strand DNA or RNA ligases and single-strand polynucleotide ligases, for example any of the ligases described in EC 6.5.1.1 (ATP-dependent ligases), EC 6.5.1.2 (NAD+−dependent ligases), EC 6.5.1.3 (RNA ligases). Specific examples of ligases comprise bacterial ligases such as E. coli DNA ligase, Tth DNA ligase, Thermococcus sp. (strain 9° N) DNA ligase (9° N™ DNA ligase, New England Biolabs), Taq DNA ligase, Ampligase™ (Epicentre Biotechnologies) and phage ligases such as T3 DNA ligase, T4 DNA ligase and T7 DNA ligase and mutants thereof. In some embodiments, the ligase is a T4 RNA ligase. In some embodiments, the ligase is a splintR ligase. In some embodiments, the ligase is a single stranded DNA ligase. In some embodiments, the ligase is a T4 DNA ligase. In some embodiments, the ligase is a ligase that has an DNA-splinted DNA ligase activity. In some embodiments, the ligase is a ligase that has an RNA-splinted DNA ligase activity.

In some embodiments, the ligation herein is a direct ligation. In some embodiments, the ligation herein is an indirect ligation. “Direct ligation” means that the ends of the polynucleotides hybridize immediately adjacently to one another to form a substrate for a ligase enzyme resulting in their ligation to each other (intramolecular ligation). Alternatively, “indirect” means that the ends of the polynucleotides hybridize non-adjacently to one another, e.g., separated by one or more intervening nucleotides or “gaps”. In some embodiments, said ends are not ligated directly to each other, but instead occurs either via the intermediacy of one or more intervening (so-called “gap” or “gap-filling” (oligo)nucleotides) or by the extension of the 3′ end of a probe to “fill” the “gap” corresponding to said intervening nucleotides (intermolecular ligation). In some cases, the gap of one or more nucleotides between the hybridized ends of the polynucleotides may be “filled” by one or more “gap” (oligo)nucleotide(s) which are complementary to a splint, circularizable probe (e.g., padlock probe), or target nucleic acid. The gap may be a gap of 1 to 60 nucleotides or a gap of 1 to 40 nucleotides or a gap of 3 to 40 nucleotides. In specific embodiments, the gap may be a gap of about 1, 2, 3, 4, 5, 6, 7, 8, 9 or 10 or more nucleotides of any integer (or range of integers) of nucleotides in between the indicated values. In some embodiments, the gap between said terminal regions may be filled by a gap oligonucleotide or by extending the 3′ end of a polynucleotide. In some cases, ligation involves ligating the ends of the probe to at least one gap (oligo)nucleotide, such that the gap (oligo)nucleotide becomes incorporated into the resulting polynucleotide. In some embodiments, the ligation herein is preceded by gap filling. In other embodiments, the ligation herein does not require gap filling.

In some embodiments, the method comprises detecting a product that includes a molecule or a complex generated in a series of reactions, e.g., hybridization, ligation, extension, replication, transcription/reverse transcription, and/or amplification (e.g., rolling circle amplification), in any suitable combination.

In some aspects, the polynucleotides and/or amplification product (e.g., amplicon) can be anchored to a polymer matrix. For example, the polymer matrix can be a hydrogel. In some embodiments, one or more of the polynucleotide probes can be modified to contain functional groups that can be used as an anchoring site to attach the polynucleotide probes and/or amplification product to a polymer matrix. Exemplary modification and polymer matrix that can be employed in accordance with the provided embodiments comprise those described in, for example, WO 2014/163886, WO 2017/079406, US 2016/0024555, US 2018/0251833 and US 2017/0219465. In some examples, the scaffold also contains modifications or functional groups that can react with or incorporate the modifications or functional groups of the probe set or amplification product. In some examples, the scaffold can comprise oligonucleotides, polymers or chemical groups, to provide a matrix and/or support structures.

The amplification products may be immobilized within the matrix generally at the location of the nucleic acid being amplified, thereby creating a localized colony of amplicons. The amplification products may be immobilized within the matrix by steric factors. The amplification products may also be immobilized within the matrix by covalent or noncovalent bonding. In this manner, the amplification products may be considered to be attached to the matrix. By being immobilized to the matrix, such as by covalent bonding or cross-linking, the size and spatial relationship of the original amplicons is maintained. By being immobilized to the matrix, such as by covalent bonding or cross-linking, the amplification products are resistant to movement or unraveling under mechanical stress.

In some aspects, the amplification products are copolymerized and/or covalently attached to the surrounding matrix thereby preserving their spatial relationship and any information inherent thereto. For example, if the amplification products are those generated from DNA or RNA within a cell embedded in the matrix, the amplification products can also be functionalized to form covalent attachment to the matrix preserving their spatial information within the cell thereby providing a subcellular localization distribution pattern. In some embodiments, the provided methods involve embedding the one or more polynucleotide probe sets and/or the amplification products in the presence of hydrogel subunits to form one or more hydrogel-embedded amplification products. In some embodiments, the hydrogel-tissue chemistry described comprises covalently attaching nucleic acids to in situ synthesized hydrogel for tissue clearing, enzyme diffusion, and multiple-cycle sequencing while an existing hydrogel-tissue chemistry method cannot. In some embodiments, to enable amplification product embedding in the tissue-hydrogel setting, amine-modified nucleotides are comprised in the amplification step (e.g., RCA), functionalized with an acrylamide moiety using acrylic acid N-hydroxysuccinimide esters, and copolymerized with acrylamide monomers to form a hydrogel.

In some embodiments, a method disclosed herein may also comprise one or more signal amplification components. In some embodiments, the present disclosure relates to the detection of nucleic acids sequences in situ using probe hybridization and generation of amplified signals associated with the probes, wherein background signal is reduced and sensitivity is increased. In some embodiments, tethering of fragmented RNA (e.g., as described in Section II) can increase sensitivity and reduce the need for using multiple probes per analyte. In some embodiments, an analyte disclosed herein can be detected in with a method that comprises signal amplification. Exemplary signal amplification methods include targeted deposition of detectable reactive molecules around the site of probe hybridization, targeted assembly of branched structures (e.g., bDNA or branched assay using locked nucleic acid (LNA)), programmed in situ growth of concatemers by enzymatic rolling circle amplification (RCA) (e.g., as described in US 2019/0055594 incorporated herein by reference), hybridization chain reaction, assembly of topologically catenated DNA structures using serial rounds of chemical ligation (clampFISH), signal amplification via hairpin-mediated concatemerization (e.g., as described in US 2020/0362398 incorporated herein by reference), e.g., primer exchange reactions such as signal amplification by exchange reaction (SABER) or SABER with DNA-Exchange (Exchange-SABER). In some embodiments, a non-enzymatic signal amplification method may be used.

In some embodiments, the method provided herein comprises contacting the biological sample with a probe or probe set that binds directly or indirectly to the ribonucleic acid and generating an amplification product using the probe or probe set. For example, an RCA product can be generated using a circular or circularizable probe as a template. In some instances, a primer oligonucleotide is added for amplification. In some instances, the primer oligonucleotide is added with a circularizable probe or probe set (e.g., for RCA). In some instances, the primer oligonucleotide is added before or after the circularizable probe or probe set is contacted with the sample. In some instances, the primer oligonucleotide for amplification of the circularized probe may comprise a sequence complementary to a target nucleic acid, as well as a sequence complementary to the circularizable probe that hybridizes to the target nucleic acid. In some embodiments, a washing step is performed to remove any unbound probes, primers, etc. In some embodiments, the wash is a stringency wash. Washing steps can be performed at any point during the process to remove non-specifically bound probes, probes that have ligated, etc.

A primer oligonucleotide for amplification of the circularized probe can comprise a single-stranded nucleic acid sequence having a 3′ end that can be used as a substrate for a nucleic acid polymerase in a nucleic acid extension reaction. The primer oligonucleotide can comprise both RNA nucleotides and DNA nucleotides (e.g., in a random or designed pattern). The primer oligonucleotide can also comprise other natural or synthetic nucleotides described herein that can have additional functionality. The primer oligonucleotide can be about 6 bases to about 100 bases, such as about 25 bases.

In some instances, upon addition of a DNA polymerase in the presence of appropriate dNTP precursors and other cofactors, the amplification primer is elongated by replication of multiple copies of the template. The amplification step can utilize isothermal amplification or non-isothermal amplification. In some embodiments, after the formation of the hybridization complex and any subsequent circularization (such as ligation of, e.g., a circularizable probe), the circularized probe is rolling-circle amplified to generate a RCA product (e.g., amplicon) containing multiple copies of the sequence of the circularized probe.

In some embodiments, RCPs are generated using a polymerase selected from the group consisting of Phi29 DNA polymerase, Phi29-like DNA polymerase, M2 DNA polymerase, B103 DNA polymerase, GA-1 DNA polymerase, phi-PRD1 polymerase, Vent DNA polymerase, Deep Vent DNA polymerase, Vent (exo-) DNA polymerase, KlenTaq DNA polymerase, DNA polymerase I, Klenow fragment of DNA polymerase I, DNA polymerase III, T3 DNA polymerase, T4 DNA polymerase, T5 DNA polymerase, T7 DNA polymerase, Bst polymerase, rBST DNA polymerase, N29 DNA polymerase, TopoTaq DNA polymerase, T7 RNA polymerase, SP6 RNA polymerase, T3 RNA polymerase, and a variant or derivative thereof. In some embodiments, the polymerase is Phi29 DNA polymerase.

The detectable reactive molecules may comprise tyramide, such as used in tyramide signal amplification (TSA) or multiplexed catalyzed reporter deposition (CARD)-FISH. In some embodiments, the detectable reactive molecule may be releasable and/or cleavable from a detectable label such as a fluorophore. In some embodiments, a method disclosed herein comprises multiplexed analysis of a biological sample comprising consecutive cycles of probe hybridization, fluorescence imaging, and signal removal, where the signal removal comprises removing the fluorophore from a fluorophore-labeled reactive molecule (e.g., tyramide). Exemplary detectable reactive reagents and methods are described in U.S. Pat. No. 6,828,109, US 2019/0376956, US 2019/0376956, WO 2020/102094, WO 2020/163397, and WO 2021/067475, all of which are incorporated herein by reference in their entireties.

In some embodiments, hybridization chain reaction (HCR) can be used for in situ signal amplification and detection. HCR is an enzyme-free nucleic acid amplification based on a triggered chain of hybridization of nucleic acid molecules starting from HCR monomers, which hybridize to one another to form a nicked nucleic acid polymer. This polymer is the product of the HCR reaction which is ultimately detected in order to indicate the presence of the target reporter oligonucleotide. HCR is described in detail in Dirks and Pierce, 2004, PNAS, 101(43), 15275-15278 and in U.S. Pat. Nos. 7,632,641 and 7,721,721 (see also US 2006/00234261; Chemeris et al, 2008 Doklady Biochemistry and Biophysics, 419, 53-55; Niu et al, 2010, 46, 3089-3091; Choi et al, 2010, Nat. Biotechnol. 28(11), 1208-1212; and Song et al, 2012, Analyst, 137, 1396-1401). HCR monomers typically comprise a hairpin, or other metastable nucleic acid structure. In the simplest form of HCR, two different types of stable hairpin monomer, referred to here as first and second HCR monomers, undergo a chain reaction of hybridization events to form a long nicked double-stranded DNA molecule when an “initiator” nucleic acid molecule is introduced. The HCR monomers have a hairpin structure comprising a double stranded stem region, a loop region connecting the two strands of the stem region, and a single stranded region at one end of the double stranded stem region. The single stranded region which is exposed (and which is thus available for hybridization to another molecule, e.g. initiator or other HCR monomer) when the monomers are in the hairpin structure may be known as the “toehold region” (or “input domain”). The first HCR monomers each further comprise a sequence which is complementary to a sequence in the exposed toehold region of the second HCR monomers. This sequence of complementarity in the first HCR monomers may be known as the “interacting region” (or “output domain”). Similarly, the second HCR monomers each comprise an interacting region (output domain), e.g. a sequence which is complementary to the exposed toehold region (input domain) of the first HCR monomers. In the absence of the HCR initiator, these interacting regions are protected by the secondary structure (e.g. they are not exposed), and thus the hairpin monomers are stable or kinetically trapped (also referred to as “metastable”) and remain as monomers (e.g. preventing the system from rapidly equilibrating), because the first and second sets of HCR monomers cannot hybridize to each other. However, once the initiator is introduced, it is able to hybridize to the exposed toehold region of a first HCR monomer, and invade it, causing it to open up. This exposes the interacting region of the first HCR monomer (e.g. the sequence of complementarity to the toehold region of the second HCR monomers), allowing it to hybridize to and invade a second HCR monomer at the toehold region. This hybridization and invasion in turn opens up the second HCR monomer, exposing its interacting region (which is complementary to the toehold region of the first HCR monomers), and allowing it to hybridize to and invade another first HCR monomer. The reaction continues in this manner until all of the HCR monomers are exhausted (e.g. all of the HCR monomers are incorporated into a polymeric chain). Ultimately, this chain reaction leads to the formation of a nicked chain of alternating units of the first and second monomer species. The presence of the HCR initiator is thus required in order to trigger the HCR reaction by hybridization to and invasion of a first HCR monomer. The first and second HCR monomers are designed to hybridize to one another are thus may be defined as cognate to one another. They are also cognate to a given HCR initiator sequence. HCR monomers which interact with one another (hybridize) may be described as a set of HCR monomers or an HCR monomer, or hairpin, system.

An HCR reaction could be carried out with more than two species or types of HCR monomers. For example, a system involving three HCR monomers could be used. In such a system, each first HCR monomer may comprise an interacting region which binds to the toehold region of a second HCR monomer; each second HCR may comprise an interacting region which binds to the toehold region of a third HCR monomer; and each third HCR monomer may comprise an interacting region which binds to the toehold region of a first HCR monomer. The HCR polymerization reaction would then proceed as described above, except that the resulting product would be a polymer having a repeating unit of first, second and third monomers consecutively. Corresponding systems with larger numbers of sets of HCR monomers could readily be conceived.

### (b) Analysis

A target sequence for a probe or probe set disclosed herein may be comprised in any analyte disclose herein, including an endogenous analyte (e.g., a probe or probe set that hybridizes directly or indirectly to the ribonucleic acid), a labelling agent, or a product or derivative of an endogenous analyte and/or a labelling agent.

In some aspects, one or more of the target sequences includes one or more barcode(s), e.g., at least two, three, four, five, six, seven, eight, nine, ten, or more barcodes. Barcodes can spatially-resolve molecular components found in biological samples, for example, within a cell or a tissue sample. A barcode can be attached to an analyte or to another moiety or structure in a reversible or irreversible manner. A barcode can be added to, for example, a fragment of a deoxyribonucleic acid (DNA) or ribonucleic acid (RNA) sample before or during sequencing of the sample. Barcodes can allow for identification and/or quantification of individual sequencing-reads (e.g., a barcode can be or can include a unique molecular identifier or “UMI”). In some aspects, a barcode comprises about 4, 5, 6, 7, 8, 9, 10, 11, 12, 13, 14, 15, 16, 17, 18, 19, 20, 21, 22, 23, 24, 25, 26, 27, 28, 29, 30, or more than 30 nucleotides.

In some embodiments, a barcode includes two or more sub-barcodes that together function as a single barcode. For example, a polynucleotide barcode can include two or more polynucleotide sequences (e.g., sub-barcodes) that are separated by one or more non-barcode sequences. In some embodiments, the teow or more sub-barcodes can be overlapping sequences. In some embodiments, the one or more barcode(s) can also provide a platform for targeting functionalities, such as oligonucleotides, oligonucleotide-antibody conjugates, oligonucleotide-streptavidin conjugates, modified oligonucleotides, affinity purification, detectable moieties, enzymes, enzymes for detection assays or other functionalities, and/or for detection and identification of the polynucleotide.

In any of the preceding embodiments, barcodes (e.g., primary and/or secondary barcode sequences) can be analyzed (e.g., detected or sequenced) using any suitable methods or techniques, including those described herein, such as RNA sequential probing of targets (RNA SPOTs), sequential fluorescent in situ hybridization (seqFISH), single-molecule fluorescent in situ hybridization (smFISH), multiplexed error-robust fluorescence in situ hybridization (MERFISH), in situ sequencing, hybridization-based in situ sequencing (HybISS), targeted in situ sequencing, fluorescent in situ sequencing (FISSEQ), sequencing by synthesis (SBS), sequencing by ligation (SBL), sequencing by hybridization (SBH), or spatially-resolved transcript amplicon readout mapping (STARmap). In any of the preceding embodiments, the methods provided herein can include analyzing the barcodes by sequential hybridization and detection with a plurality of labelled probes (e.g., detection oligos).

In some embodiments, in a barcode sequencing method, barcode sequences are detected for identification of other molecules including nucleic acid molecules (DNA or RNA) longer than the barcode sequences themselves, as opposed to direct sequencing of the longer nucleic acid molecules. In some embodiments, a N-mer barcode sequence comprises 4^{N} complexity given a sequencing read of N bases, and a much shorter sequencing read may be required for molecular identification compared to non-barcode sequencing methods such as direct sequencing. For example, 1024 molecular species may be identified using a 5-nucleotide barcode sequence (45=1024), whereas 8 nucleotide barcodes can be used to identify up to 65,536 molecular species, a number greater than the total number of distinct genes in the human genome. In some embodiments, the barcode sequences rather than endogenous sequences, which can be an efficient read-out in terms of information per cycle of sequencing. Because the barcode sequences are pre-determined, they can also be designed to feature error detection and correction mechanisms, see, e.g., U.S. Pat. Pub. 20190055594 and US 2021/0164039, which are hereby incorporated by reference in their entirety.

In some embodiments, all or a portion of the RCP (e.g., a barcode sequence in the RCP) is detected using a base-by-base sequencing method, e.g., SBS or SBB. In some embodiments, the biological sample is contacted with a sequencing primer and base-by-base sequencing using a cyclic series of nucleotide incorporation or binding, respectively, thereby generating extension products of the sequencing primer is performed followed by removing, cleaving, or blocking the extension products of the sequencing primer.

In some embodiments, the base-by-base sequencing comprises using a polymerase that is fluorescently labeled. In some embodiments, the base-by-base sequencing comprises using a polymerase-nucleotide conjugate comprising a fluorescently labeled polymerase linked to a nucleotide moiety that is not fluorescently labeled. In some embodiments, the base-by-base sequencing comprises using a multivalent polymer-nucleotide conjugate comprising a polymer core, multiple nucleotide moieties, and one or more fluorescent labels.

In some embodiments, sequencing can be performed by sequencing-by-synthesis (SBS). In some embodiments, a sequencing primer is complementary to sequences at or near the one or more barcode(s). In such embodiments, sequencing-by-synthesis can comprise reverse transcription and/or amplification in order to generate a template sequence from which a primer sequence can bind. Example SBS methods comprise those described for example, but not limited to, US 2007/0166705, US 2006/0188901, U.S. Pat. No. 7,057,026, US 2006/0240439, US 2006/0281109, US 2011/0059865, US 2005/0100900, U.S. Pat. No. 9,217,178, US 2009/0118128, US 2012/0270305, US 2013/0260372, and US 2013/0079232, all of which are herein incorporated by reference in their entireties.

In some embodiments, sequencing can be performed by sequencing-by-binding (SBB). Various aspects of SBB are described in U.S. Pat. No. 10,655,176 B2, the content of which is herein incorporated by reference in its entirety. In some embodiments, SBB comprises performing repetitive cycles of detecting a stabilized complex that forms at each position along the template nucleic acid to be sequenced (e.g. a ternary complex that includes the primed template nucleic acid, a polymerase, and a cognate nucleotide for the position), under conditions that prevent covalent incorporation of the cognate nucleotide into the primer, and then extending the primer to allow detection of the next position along the template nucleic acid. In the sequencing-by-binding approach, detection of the nucleotide at each position of the template occurs prior to extension of the primer to the next position. Generally, the methodology is used to distinguish the four different nucleotide types that can be present at positions along a nucleic acid template by uniquely labelling each type of ternary complex (i.e. different types of ternary complexes differing in the type of nucleotide it contains) or by separately delivering the reagents needed to form each type of ternary complex. In some instances, the labeling may comprise fluorescence labeling of, e.g., the cognate nucleotide or the polymerase that participate in the ternary complex.

In some embodiments, sequencing can be performed by sequencing-by-avidity (SBA). Some aspects of SBA approaches are described in U.S. Pat. No. 10,768,173 B2, the content of which is herein incorporated by reference in its entirety. In some embodiments, SBA comprises detecting a multivalent binding complex formed between a fluorescently-labeled polymer-nucleotide conjugate, and a one or more primed target nucleic acid sequences (e.g., barcode sequences). Fluorescence imaging is used to detect the bound complex and thereby determine the identity of the N+1 nucleotide in the target nucleic acid sequence (where the primer extension strand is N nucleotides in length). Following the imaging step, the multivalent binding complex is disrupted and washed away, the correct blocked nucleotide is incorporated into the primer extension strand, and the sequencing cycle is repeated.

In some embodiments, detection of the barcode sequences is performed by sequential hybridization of probes to the barcode sequences or complements thereof and detecting complexes formed by the probes and barcode sequences or complements thereof. In some cases, each barcode sequence or complement thereof is assigned a sequence of signal codes that identifies the barcode sequence or complement thereof (e.g., a temporal signal signature or code that identifies the analyte), and detecting the barcode sequences or complements thereof can comprise decoding the barcode sequences of complements thereof by detecting the corresponding sequences of signal codes detected from sequential hybridization, detection, and removal of sequential pools of intermediate probes and the universal pool of detectably labeled probes. In some cases, the sequences of signal codes can be fluorophore sequences assigned to the corresponding barcode sequences or complements thereof. In some embodiments, the detectably labeled probes are fluorescently labeled. In some embodiments, the barcode sequence or complement thereof is performed by sequential probe hybridization as described in US 2021/0340618, the content of which is herein incorporated by reference in its entirety.

In any of the embodiments herein, the detecting step can comprise contacting the biological sample with one or more detectably labeled probes that directly or indirectly hybridize to the barcode sequences or complements thereof (e.g., in amplification products generated using the probes or probe sets), and dehybridizing the one or more detectably labeled probes. In any of the embodiments herein, the contacting and dehybridizing steps can be repeated with the one or more detectably labeled probes and/or one or more other detectably labeled probes that directly or indirectly hybridize to the barcode sequences or complements thereof. In some aspects, the method comprises sequential hybridization of detectably labeled probes to create a spatiotemporal signal signature or code that identifies the analyte.

In any of the embodiments herein, the detecting step can comprise contacting the biological sample with one or more first detectably labeled probes that directly hybridize to the plurality of probes or probe sets. In some instances, the detecting step can comprise contacting the biological sample with one or more first detectably labeled probes that indirectly hybridize to the plurality of probes or probe sets. In any of the embodiments herein, the detecting step can comprise contacting the biological sample with one or more first detectably labeled probes that directly or indirectly hybridize to the plurality of probes or probe sets.

In any of the embodiments herein, the detecting step can comprise contacting the biological sample with one or more intermediate probes that directly or indirectly hybridize to the barcode sequences or complements thereof (e.g., of the plurality of probes or probe sets or rolling circle amplification product generated using the plurality of probes or probe sets), wherein the one or more intermediate probes are detectable using one or more detectably labeled probes. In any of the embodiments herein, the detecting step can further comprise dehybridizing the one or more intermediate probes and/or the one or more detectably labeled probes from the barcode sequences or complements thereof (e.g., of the plurality of probes or probe sets or rolling circle amplification product generated using the plurality of probes or probe sets). In any of the embodiments herein, the contacting and dehybridizing steps can be repeated with the one or more intermediate probes, the one or more detectably labeled probes, one or more other intermediate probes, and/or one or more other detectably labeled probes.

In some embodiments, sequence detection can be performed using single molecule sequencing by ligation. Such techniques utilize DNA ligase to incorporate oligonucleotides and identify the incorporation of such oligonucleotides. The oligonucleotides typically have different labels that are correlated with the identity of a particular nucleotide in a sequence to which the oligonucleotides hybridize. Aspects and features involved in sequencing by ligation are described, for example, in Shendure et al. Science (2005), 309: 1728-1732, and in U.S. Pat. Nos. 5,599,675; 5,750,341; 6,969,488; 6,172,218; and 6,306,597, all of which are herein incorporated by reference in their entireties.

In some embodiments, nucleic acid hybridization can be used for detecting the analytes. These methods utilize labeled nucleic acid probes that are complementary to at least a portion of a barcode sequence. Multiplex decoding can be performed with pools of many different probes with distinguishable labels. Non-limiting examples of nucleic acid hybridization sequencing are described for example in U.S. Pat. No. 8,460,865, and in Gunderson et al., Genome Research 14:870-877 (2004), all of which are herein incorporated by reference in their entireties.

In some embodiments, a probe or probe set can be a probe comprising a 3′ or 5′ overhang upon hybridization to the target nucleic acid (e.g., an L-shaped intermediate probe). In some embodiments, the overhang comprises one or more barcode sequences corresponding to the target nucleic acid (e.g., the target RNA transcript). In some embodiments, a plurality of probes are designed to hybridize to the target nucleic acid (e.g., at least 20, 30, or 40 probes can hybridize to the target nucleic acid). In some embodiments, the probe or probe set is a probe comprising a 3′ overhang and a 5′ overhang upon hybridization to the target nucleic acid (a U-shaped probe). In some embodiments, the 3′ overhang and the 5′ overhang each independently comprises one or more detectable labels and/or barcode sequences. In some embodiments, the 3′ and/or 5′ overhang comprises one or more detectable labels and/or barcode sequences.

In some embodiments, analysis comprises using a codebook comprising signal code sequence that are sequences of color codes, arranged in the order of the corresponding signal color detected in sequential cycles of probe hybridization and imaging. In some aspects, the provided methods which immobilize and tether RNA in the biological sample can be advantageous when using detection methods that comprise a plurality of repeated cycles of hybridization and removal of probes (e.g., detectably labeled probes, or intermediate probes that bind to detectably labeled probes) to the primary probe or probe set hybridized to the RNA, or to a rolling circle amplification product generated from the probe or probe set hybridized to the RNA.

### IV. Kits

In some aspects, provided herein are kits for analyzing fragmented nucleic acids in a biological sample. In some embodiments, provided herein are kits for analyzing a ribonucleic acid in a biological sample embedded in a three-dimensional polymerized matrix according to any of the methods described herein. In some embodiments, provided herein is a kit comprising (a) a 3′ phosphatase, optionally wherein the 3′ phosphatase is T4 polynucleotide kinase; (b) a formylation reagent, optionally wherein the reagent is an oxidant, optionally wherein the oxidant is sodium metaperiodate; (c) an attachment agent comprising a reactive group capable of reacting with an aldehyde moiety and an attachment moiety capable of attaching covalently or non-covalently to an exogenous or endogenous molecule in the biological sample, optionally wherein the kit further comprises one or more reagents for reacting the attachment agent with the aldehyde; (d) an exogenous molecule capable of attaching covalently or non-covalently to the attachment moiety, optionally wherein the exogenous molecule is a matrix-forming agent for embedding the biological sample in a three-dimensional polymerized matrix; (e) a clearing agent, optionally wherein the clearing agent is a detergent, a lipase, and/or a protease; and (f) instructions for use.

In some embodiments, the kit further comprises a probe or probe set designed to hybridize to a fragmented nucleic acid. In some aspects, the probe or probe set that binds directly or indirectly to the ribonucleic acid is provided in a separate kit. In some cases, further provided is a kit comprising reagents for detecting the probe or probe set.

The various components of the kit may be present in separate containers or certain compatible components may be pre-combined into a single container. In some embodiments, the kits further contain instructions for using the components of the kit to practice the provided methods.

In some embodiments, the kits can contain reagents and/or consumables required for performing one or more steps of the provided methods. In some embodiments, the kits contain reagents for fixing, embedding, and/or permeabilizing the biological sample. In some embodiments, the kits contain reagents, such as enzymes and buffers for ligation and/or amplification, such as ligases and/or polymerases. In some aspects, provided is a kit that comprises any of the reagents described herein, e.g., wash buffer and ligation buffer. In some embodiments, provided is a kit that contain reagents for detection and/or sequencing, such as barcode detection probes or detectable labels. In some embodiments, the kits optionally contain other components, for example nucleic acid primers, enzymes and reagents, buffers, nucleotides, modified nucleotides, reagents for additional assays.

### V. Terminology

Specific terminology is used throughout this disclosure to explain various aspects of the apparatus, systems, methods, and compositions that are described.

Having described some illustrative embodiments of the present disclosure, it should be apparent to those skilled in the art that the foregoing is merely illustrative and not limiting, having been presented by way of example only. Numerous modifications and other illustrative embodiments are within the scope of one of ordinary skill in the art and are contemplated as falling within the scope of the present disclosure. In particular, although many of the examples presented herein involve specific combinations of method acts or system elements, it should be understood that those acts and those elements may be combined in other ways to accomplish the same objectives.

As used herein, the singular forms “a,” “an,” and “the” include plural referents unless the context clearly dictates otherwise. For example, “a” or “an” means “at least one” or “one or more.”

The term “about” as used herein refers to the usual error range for the respective value in this technical field. Reference to “about” a value or parameter herein includes (and describes) embodiments that are directed to that value or parameter per se.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

### A. Barcode

A “barcode” is a label, or identifier, which conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes.

Barcodes can have a variety of different formats. For example, barcodes can include polynucleotide barcodes, random nucleic acid and/or amino acid sequences, and synthetic nucleic acid and/or amino acid sequences. A barcode can be attached to an analyte or to another moiety or structure in a reversible or irreversible manner. A barcode can be added to, for example, a fragment of a deoxyribonucleic acid (DNA) or ribonucleic acid (RNA) sample before or during sequencing of the sample. Barcodes can allow for identification and/or quantification of individual sequencing-reads (e.g., a barcode can be or can include a unique molecular identifier or “UMI”).

Barcodes can spatially-resolve molecular components found in biological samples, for example, at single-cell resolution (e.g., a barcode can be or can include a “spatial barcode”). In some embodiments, a barcode includes both a UMI and a spatial barcode. In some embodiments, a barcode includes two or more sub-barcodes that together function as a single barcode. For example, a polynucleotide barcode can include two or more polynucleotide sequences (e.g., sub-barcodes) that are separated by one or more non-barcode sequences.

### B. Nucleic Acid and Nucleotide

The terms “nucleic acid” and “nucleotide” are intended to be consistent with their use in the art and to include naturally-occurring species or functional analogs thereof. Particularly useful functional analogs of nucleic acids are capable of hybridizing to a nucleic acid in a sequence-specific fashion (e.g., capable of hybridizing to two nucleic acids such that ligation can occur between the two hybridized nucleic acids) or are capable of being used as a template for replication of a particular nucleotide sequence. Naturally-occurring nucleic acids generally have a backbone containing phosphodiester bonds. An analog structure can have an alternate backbone linkage. Naturally-occurring nucleic acids generally have a deoxyribose sugar (e.g., found in deoxyribonucleic acid (DNA)) or a ribose sugar (e.g. found in ribonucleic acid (RNA)).

A nucleic acid can contain nucleotides having any of a variety of analogs of these sugar moieties. A nucleic acid can include native or non-native nucleotides. In this regard, a native deoxyribonucleic acid can have one or more bases selected from the group consisting of adenine (A), thymine (T), cytosine (C), or guanine (G), and a ribonucleic acid can have one or more bases selected from the group consisting of uracil (U), adenine (A), cytosine (C), or guanine (G).

### C. Probe and Target

A “probe” or a “target,” when used in reference to a nucleic acid or sequence of a nucleic acids, is intended as a semantic identifier for the nucleic acid or sequence in the context of a method or composition and does not limit the structure or function of the nucleic acid or sequence beyond what is expressly indicated.

### D. Oligonucleotide and Polynucleotide

The terms “oligonucleotide” and “polynucleotide” are used interchangeably to refer to a single-stranded multimer of nucleotides from about 2 to about 500 nucleotides in length. Oligonucleotides can be synthetic, made enzymatically (e.g., via polymerization), or using a “split-pool” method. Oligonucleotides can include ribonucleotide monomers (e.g., can be oligoribonucleotides) and/or deoxyribonucleotide monomers (e.g., oligodeoxyribonucleotides). In some examples, oligonucleotides can include a combination of both deoxyribonucleotide monomers and ribonucleotide monomers in the oligonucleotide (e.g., random or ordered combination of deoxyribonucleotide monomers and ribonucleotide monomers). An oligonucleotide can be 4 to 10, 10 to 20, 21 to 30, 31 to 40, 41 to 50, 51 to 60, 61 to 70, 71 to 80, 80 to 100, 100 to 150, 150 to 200, 200 to 250, 250 to 300, 300 to 350, 350 to 400, or 400-500 nucleotides in length, for example. Oligonucleotides can include one or more functional moieties that are attached (e.g., covalently or non-covalently) to the multimer structure. For example, an oligonucleotide can include one or more detectable labels (e.g., a radioisotope or fluorophore).

### E. Hybridizing, Hybridize, Annealing, and Anneal

The terms “hybridizing,” “hybridize,” “annealing,” and “anneal” are used interchangeably in this disclosure and refer to the pairing of substantially complementary or complementary nucleic acid sequences within two different molecules. Pairing can be achieved by any process in which a nucleic acid sequence joins with a substantially or fully complementary sequence through base pairing to form a hybridization complex. For purposes of hybridization, two nucleic acid sequences are “substantially complementary” if at least 60% (e.g., at least 70%, at least 80%, or at least 90%) of their individual bases are complementary to one another.

### F. Primer

A “primer” is a single-stranded nucleic acid sequence having a 3′ end that can be used as a substrate for a nucleic acid polymerase in a nucleic acid extension reaction. RNA primers are formed of RNA nucleotides, and are used in RNA synthesis, while DNA primers are formed of DNA nucleotides and used in DNA synthesis. Primers can also include both RNA nucleotides and DNA nucleotides (e.g., in a random or designed pattern). Primers can also include other natural or synthetic nucleotides described herein that can have additional functionality. In some examples, DNA primers can be used to prime RNA synthesis and vice versa (e.g., RNA primers can be used to prime DNA synthesis). Primers can vary in length. For example, primers can be about 6 bases to about 120 bases. For example, primers can include up to about 25 bases. A primer, may in some cases, refer to a primer binding sequence.

### G. Primer Extension

A “primer extension” refers to any method where two nucleic acid sequences (e.g., a constant region from each of two distinct capture probes) become linked (e.g., hybridized) by an overlap of their respective terminal complementary nucleic acid sequences (e.g., 3′ termini). Such linking can be followed by nucleic acid extension (e.g., an enzymatic extension) of one, or both termini using the other nucleic acid sequence as a template for extension. Enzymatic extension can be performed by an enzyme including, but not limited to, a polymerase and/or a reverse transcriptase.

### H. Nucleic Acid Extension

A “nucleic acid extension” generally involves incorporation of one or more nucleic acids (e.g., A, G, C, T, U, nucleotide analogs, or derivatives thereof) into a molecule (such as, but not limited to, a nucleic acid sequence) in a template-dependent manner, such that consecutive nucleic acids are incorporated by an enzyme (such as a polymerase or reverse transcriptase), thereby generating a newly synthesized nucleic acid molecule. For example, a primer that hybridizes to a complementary nucleic acid sequence can be used to synthesize a new nucleic acid molecule by using the complementary nucleic acid sequence as a template for nucleic acid synthesis. Similarly, a 3′ polyadenylated tail of an mRNA transcript that hybridizes to a poly (dT) sequence (e.g., capture domain) can be used as a template for single-strand synthesis of a corresponding cDNA molecule.

### I. Antibody

An “antibody” is a polypeptide molecule that recognizes and binds to a complementary target antigen. Antibodies typically have a molecular structure shape that resembles a Y shape. Naturally-occurring antibodies, referred to as immunoglobulins, belong to one of the immunoglobulin classes IgG, IgM, IgA, IgD, and IgE. Antibodies can also be produced synthetically. For example, recombinant antibodies, which are monoclonal antibodies, can be synthesized using synthetic genes by recovering the antibody genes from source cells, amplifying into an appropriate vector, and introducing the vector into a host to cause the host to express the recombinant antibody. In general, recombinant antibodies can be cloned from any species of antibody-producing animal using suitable oligonucleotide primers and/or hybridization probes. Recombinant techniques can be used to generate antibodies and antibody fragments, including non-endogenous species.

Synthetic antibodies can be derived from non-immunoglobulin sources. For example, antibodies can be generated from nucleic acids (e.g., aptamers), and from non-immunoglobulin protein scaffolds (such as peptide aptamers) into which hypervariable loops are inserted to form antigen binding sites. Synthetic antibodies based on nucleic acids or peptide structures can be smaller than immunoglobulin-derived antibodies, leading to greater tissue penetration.

Antibodies can also include affimer proteins, which are affinity reagents that typically have a molecular weight of about 12-14 kDa. Affimer proteins generally bind to a target (e.g., a target protein) with both high affinity and specificity. Examples of such targets include, but are not limited to, ubiquitin chains, immunoglobulins, and C-reactive protein. In some embodiments, affimer proteins are derived from cysteine protease inhibitors, and include peptide loops and a variable N-terminal sequence that provides the binding site.

Antibodies can also refer to an “epitope binding fragment” or “antibody fragment,” which as used herein, generally refers to a portion of a complete antibody capable of binding the same epitope as the complete antibody, albeit not necessarily to the same extent. Although multiple types of epitope binding fragments are possible, an epitope binding fragment typically comprises at least one pair of heavy and light chain variable regions (VH and VL, respectively) held together (e.g., by disulfide bonds) to preserve the antigen binding site and does not contain all or a portion of the Fc region. Epitope binding fragments of an antibody can be obtained from a given antibody by any suitable technique (e.g., recombinant DNA technology or enzymatic or chemical cleavage of a complete antibody), and typically can be screened for specificity in the same manner in which complete antibodies are screened. In some embodiments, an epitope binding fragment comprises an F(ab′)₂ fragment, Fab′ fragment, Fab fragment, Fd fragment, or Fv fragment. In some embodiments, the term “antibody” includes antibody-derived polypeptides, such as single chain variable fragments (scFv), diabodies or other multimeric scFvs, heavy chain antibodies, single domain antibodies, or other polypeptides comprising a sufficient portion of an antibody (e.g., one or more complementarity determining regions (CDRs)) to confer specific antigen binding ability to the polypeptide.

### J. Label, Detectable Label, and Optical Label

The terms “detectable label,” “detectable moiety”, “optical label,” and “label” are used interchangeably herein to refer to a directly or indirectly detectable moiety that is associated with (e.g., conjugated to) a molecule to be detected, e.g., a probe for in situ assay, a capture probe or analyte. The detectable label can be directly detectable by itself (e.g., radioisotope labels or fluorescent labels) or, in the case of an enzymatic label, can be indirectly detectable, e.g., by catalyzing chemical alterations of a substrate compound or composition, which substrate compound or composition is directly detectable. Detectable labels can be suitable for small scale detection and/or suitable for high-throughput screening. As such, suitable detectable labels include, but are not limited to, radioisotopes, fluorophores, chemiluminescent compounds, bioluminescent compounds, and dyes.

The detectable label can be qualitatively detected (e.g., optically or spectrally), or it can be quantified. Qualitative detection generally includes a detection method in which the existence or presence of the detectable label is confirmed, whereas quantifiable detection generally includes a detection method having a quantifiable (e.g., numerically reportable) value such as an intensity, duration, polarization, and/or other properties. In some embodiments, the detectable label is bound to a feature or to a capture probe associated with a feature. For example, detectably labelled features can include a fluorescent, a colorimetric, or a chemiluminescent label attached to a bead (see, for example, Rajeswari et al., J. MicrobiolMethods 139:22-28, 2017, and Forcucci et al., J. Biomed Opt. 10:105010, 2015, the entire contents of each of which are incorporated herein by reference).

In some embodiments, a plurality of detectable labels can be attached to a feature, capture probe, or composition to be detected. For example, detectable labels can be incorporated during nucleic acid polymerization or amplification (e.g., Cy5®-labelled nucleotides, such as Cy5®-dCTP). Any suitable detectable label can be used. In some embodiments, the detectable label is a fluorophore. For example, the fluorophore can be from a group that includes: 7-AAD (7-Aminoactinomycin D), Acridine Orange (+DNA), Acridine Orange (+RNA), Alexa Fluor® 350, Alexa Fluor® 430, Alexa Fluor® 488, Alexa Fluor® 532, Alexa Fluor® 546, Alexa Fluor® 555, Alexa Fluor® 568, Alexa Fluor® 594, Alexa Fluor® 633, Alexa Fluor® 647, Alexa Fluor® 660, Alexa Fluor® 680, Alexa Fluor® 700, Alexa Fluor® 750, Allophycocyanin (APC), AMCA/AMCA-X, 7-Aminoactinomycin D (7-AAD), 7-Amino-4-methylcoumarin, 6-Aminoquinoline, Aniline Blue, ANS, APC-Cy7, ATTO-TAG™ CBQCA, ATTO-TAG™ FQ, Auramine O-Feulgen, BCECF (high pH), BFP (Blue Fluorescent Protein), BFP/GFP FRET, BOBO™-1/BO-PRO™-1, BOBO™-3/BO-PRO™-3, BODIPY® FL, BODIPY® TMR, BODIPY® TR-X, BODIPY® 530/550, BODIPY® 558/568, BODIPY® 564/570, BODIPY® 581/591, BODIPY® 630/650-X, BODIPY® 650-665-X, BTC, Calcein, Calcein Blue, Calcium Crimson™, Calcium Green-1™ Calcium Orange™, Calcofluor® White, 5-Carboxyfluoroscein (5-FAM), 5-Carboxynaphthofluoroscein, 6-Carboxyrhodamine 6G, 5-Carboxytetramethylrhodamine (5-TAMRA), Carboxy-X-rhodamine (5-ROX), Cascade Blue®, Cascade Yellow™, CCF2 (GeneBLAzer™), CFP (Cyan Fluorescent Protein), CFP/YFP FRET, Chromomycin A3, Cl-NERF (low pH), CPM, 6-CR 6G, CTC Formazan, Cy2®, Cy3®, Cy3.5@, Cy5®, Cy5.5@, Cy7®, Cychrome (PE-Cy5), Dansylamine, Dansyl cadaverine, Dansylchloride, DAPI, Dapoxyl, DCFH, DHR, DiA (4-Di-16-ASP), DiD (DilC18(5)), DIDS, Dil (DilC18(3)), DiO (DiOC18(3)), DiR (DilC18(7)), Di-4 ANEPPS, Di-8 ANEPPS, DM-NERF (4.5-6.5 pH), DsRed (Red Fluorescent Protein), EBFP, ECFP, EGFP, ELF®-97 alcohol, Eosin, Erythrosin, Ethidium bromide, Ethidium homodimer-1 (EthD-1), Europium (III) Chloride, 5-FAM (5-Carboxyfluorescein), Fast Blue, Fluorescein-dT phosphoramidite, FITC, Fluo-3, Fluo-4, FluorX@, Fluoro-Gold™ (high pH), Fluoro-Gold™ (low pH), Fluoro-Jade, FM® 1-43, Fura-2 (high calcium), Fura-2/BCECF, Fura Red™ (high calcium), Fura Red™/Fluo-3, GeneBLAzer™ (CCF2), GFP Red Shifted (rsGFP), GFP Wild Type, GFP/BFP FRET, GFP/DsRed FRET, Hoechst 33342 & 33258, 7-Hydroxy-4-methylcoumarin (pH 9), 1,5 IAEDANS, Indo-1 (high calcium), Indo-1 (low calcium), Indodicarbocyanine, Indotricarbocyanine, JC-1, 6-JOE, JOJO™-1/JO-PRO™-1, LDS 751 (+DNA), LDS 751 (+RNA), LOLO™-1/LO-PRO™-1, Lucifer Yellow, LysoSensor™ Blue (pH 5), LysoSensor™ Green (pH 5), LysoSensor™ Yellow/Blue (pH 4.2), LysoTracker® Green, LysoTracker® Red, LysoTracker® Yellow, Mag-Fura-2, Mag-Indo-1, Magnesium Green™, Marina Blue®, 4-Methylumbelliferone, Mithramycin, MitoTracker® Green, MitoTracker® Orange, MitoTracker® Red, NBD (amine), Nile Red, Oregon Green® 488, Oregon Green® 500, Oregon Green® 514, Pacific Blue, PBF1, PE (R-phycoerythrin), PE-Cy5, PE-Cy7, PE-Texas Red, PerCP (Peridinin chlorphyll protein), PerCP-Cy5.5 (TruRed), PharRed (APC-Cy7), C-phycocyanin, R-phycocyanin, R-phycoerythrin (PE), PI (Propidium Iodide), PKH26, PKH67, POPO™-1/PO-PRO™-1, POPO™-3/PO-PRO™-3, Propidium Iodide (PI), PyMPO, Pyrene, Pyronin Y, Quantam Red (PE-Cy5), Quinacrine Mustard, R670 (PE-Cy5), Red 613 (PE-Texas Red), Red Fluorescent Protein (DsRed), Resorufin, RH 414, Rhod-2, Rhodamine B, Rhodamine Green™, Rhodamine Red™, Rhodamine Phalloidin, Rhodamine 110, Rhodamine 123, 5-ROX (carboxy-X-rhodamine), S65A, S65C, S65L, S65T, SBFI, SITS, SNAFL®-1 (high pH), SNAFL®-2, SNARF®-1 (high pH), SNARF®-1 (low pH), Sodium Green™, SpectrumAqua®, SpectrumGreen® #1, SpectrumGreen® #2, SpectrumOrange®, SpectrumRed®, SYTO® 11, SYTO® 13, SYTO® 17, SYTO® 45, SYTOX® Blue, SYTOX® Green, SYTOX® Orange, 5-TAMRA (5-Carboxytetramethylrhodamine), Tetramethylrhodamine (TRITC), Texas Red®/Texas Red®-X, Texas Red®-X (NHS Ester), Thiadicarbocyanine, Thiazole Orange, TOTO®-1/TO-PRO®-1, TOTO@-3/TO-PRO®-3, TO-PRO®-5, Tri-color (PE-Cy5), TRITC (Tetramethylrhodamine), TruRed (PerCP-Cy5.5), WW 781, X-Rhodamine (XRITC), Y66F, Y66H, Y66W, YFP (Yellow Fluorescent Protein), YOYO®-1/YO-PRO®-1, YOYO®-3/YO-PRO®-3, 6-FAM (Fluorescein), 6-FAM (NHS Ester), 6-FAM (Azide), HEX, TAMRA (NHS Ester), Yakima Yellow, MAX, TET, TEX615, ATTO 488, ATTO 532, ATTO 550, ATTO 565, ATTO Rho101, ATTO 590, ATTO 633, ATTO 647N, TYE 563, TYE 665, TYE 705, 5′ IRDye® 700, 5′ IRDye® 800, 5′ IRDye® 800CW (NHS Ester), WellRED D4 Dye, WellRED D3 Dye, WellRED D2 Dye, Lightcycler® 640 (NHS Ester), and Dy 750 (NHS Ester).

As mentioned above, in some embodiments, a detectable label is or includes a luminescent or chemiluminescent moiety. Common luminescent/chemiluminescent moieties include, but are not limited to, peroxidases such as horseradish peroxidase (HRP), soybean peroxidase (SP), alkaline phosphatase, and luciferase. These protein moieties can catalyze chemiluminescent reactions given the appropriate substrates (e.g., an oxidizing reagent plus a chemiluminescent compound. A number of compound families provide chemiluminescence under a variety of conditions. Non-limiting examples of chemiluminescent compound families include 2,3-dihydro-1,4-phthalazinedione luminol, 5-amino-6,7,8-trimethoxy- and the dimethylamino[ca]benz analog. These compounds can luminesce in the presence of alkaline hydrogen peroxide or calcium hypochlorite and base. Other examples of chemiluminescent compound families include, e.g., 2,4,5-triphenylimidazoles, para-dimethylamino and -methoxy substituents, oxalates such as oxalyl active esters, p-nitrophenyl, N-alkyl acridinum esters, luciferins, lucigenins, or acridinium esters. In some embodiments, a detectable label is or includes a metal-based or mass-based label. For example, small cluster metal ions, metals, or semiconductors may act as a mass code. In some examples, the metals can be selected from Groups 3-15 of the periodic table, e.g., Y, La, Ag, Au, Pt, Ni, Pd, Rh, Ir, Co, Cu, Bi, or a combination thereof.

### EXAMPLES

The following examples are included for illustrative purposes only and are not intended to limit the scope of the present disclosure.

### Example 1: Immobilizing Fragmented RNA

This example describes the immobilization of the RNA contained in formalin-fixed paraffin-embedded (FFPE) mouse brain (mBrain) tissue samples and subsequent embedding of the FFPE mBrain samples in a hydrogel matrix for imaging. FFPE tissue samples are known to contain degraded RNA due to the extensive processing, including baking, deparaffinization, decrosslinking and permeabilization that these samples undergo in order to facilitate analysis and imaging. In some cases, a significant amount of fragmented RNA can be lost during sample processing such as during decrosslinking of the sample, thus limiting the amount of RNA material present for imaging.

Sample Preparation: First, FFPE mBrain samples were baked at 60° C. for 2 hr and deparaffinized via (a) xylene treatment-incubation in 100% xylene twice for (2×) 10 min each, followed by (b) serial ethanol treatment-incubation with 100% ethanol (2×) 3 min each, then 96% ethanol 2×3 min each, then 70% ethanol once (1×) 3 min, followed by brief immersion in nuclease-free water (1×20 sec). The FFPE mBrain samples were not de-crosslinked or permeabilized. Next, the samples were treated with T4 polynucleotide kinase (T4 PNK) for 1 hr at 37° C. followed by a 30 min incubation at 4° C. with 5 mM sodium meta-periodate (NaIO₄).

Sample Embedding: FFPE mBrain samples were then embedded by incubating with 200 mM aniline, 150 mM triethylamine (TEA), and 100 mM 2-aminoethyl methacrylate (AEM) for 30 min at RT, followed by a 10 min incubation at RT with 100 mM sodium borohydride (NaBH₄) resuspended in water. Finally, embedding was performed using Acrylamide 4%, Bisacrylamide 0.2%, and SSC 2×. A control sample was incubated similarly with Acrylamide 4%, Bisacrylamide 0.2%, and SSC 2× without the treatment with aniline, TEA, and AEM. Samples were incubated with acrylamide, bisacrylamide and SSC 2× for 15 min followed by addition of a polymerization mixture comprising 0.2% ammonium persulphate and 0.2% tetramethylethylenediamine. The monomer buffer was allowed to polymerize for 30 min at RT. Following sample embedding, mBrain FFPE samples were cleared for 1 hr at 37° C. with 0.2 mg/mL proteinase K (PK) resuspended in PBS.

The embedded samples were contacted with a panel of probes targeting 50 mouse brain target genes, and reagents for ligation and amplification. Eight cycles of detection were performed to detect the barcode sequences associated with the probes in the amplification products. For a cycle of detection, a pool of intermediate probes targeting RCPs of the different genes were added and incubated, and fluorescently detected oligonucleotides were added to detect the intermediate probes. Different pools of intermediate probes were cycled and a universal pool of fluorescently labeled oligonucleotides were used to detect the intermediate probes in each cycle.

Through the sequential hybridization and detection cycles, fluorescent signals from the RCPs were detected and recorded at locations in the biological sample. The order of signals (or absence thereof) at a given location through the multiple cycles provided a signal code sequence for the RCP at the location, and the signal code sequence was compared to those in the codebook to identify a corresponding barcode sequence in the RCP and the gene associated therewith.

Results: Images of the samples showed numerous identifiable RCP puncta in all fluorescent channels, thereby confirming that RNA were successfully immobilized into the FFPE mBrain samples prior to embedding of the tissue samples in a hydrogel matrix and subsequent analytical work-up.

### Example 2: Immobilizing Fragmented RNA in a Human Tissue Microarray (TMA)

This example describes immobilization of RNA contained in formalin-fixed paraffin-embedded (FFPE) human tissue microarray (TMA) and subsequent embedding of the FFPE TMA samples in a hydrogel matrix for transcriptomic analysis and imaging.

Sample Preparation: First, the FFPE human TMA samples (with sectioned brain, lung, skin, colon, pancreas, liver, and kidney tissue samples) were baked at 60° C. for 2 hr and deparaffinized via (a) xylene treatment-incubation in 100% xylene twice for (2×) 10 min each, followed by (b) serial ethanol treatment-incubation with 100% ethanol (2×) 3 min each, then 96% ethanol 2×3 min each, then 70% ethanol once (1×) 3 min, followed by brief immersion in nuclease-free water (1×20 sec). The FFPE human TMA samples were not de-crosslinked or permeabilized.

For tethering the RNAs, the samples were treated with T4 polynucleotide kinase (T4 PNK) for 1 hr at 37° C. followed by a 30 min incubation at 4° C. with 5 mM sodium meta-periodate (NaIO₄). To prepare for embedding, samples were incubated with 200 mM aniline, 150 mM triethylamine (TEA), and 100 mM 2-aminoethyl methacrylate (AEM) for 30 min at RT, followed by a 10 min incubation at RT with 100 mM sodium borohydride (NaBH₄) resuspended in water. FFPE human TMA samples were then embedded by incubating with 4% Acrylamide, 0.2% Bisacrylamide, 0.2% ammonium persulfate (APS), 0.2% tetramethylethylenediamine TEMED) for 5 min. The monomer buffer was then allowed to polymerize for 30 min at RT with 60 μL gel.

The RNA tethered and hydrogel embedded (H) samples and non-embedded control (NH) samples that did not undergo RNA tethering treatment were contacted with a panel of probes targeting 377 human genes and ligation was performed by incubating with a ligase to form circularized probes. For RCA, the samples were washed and then incubated in an RCA reaction mixture (containing Phi29 reaction buffer, dNTPs, Phi29 polymerase) to generate RCA products (RCPs) using the circularized probes as template. Fifteen cycles of detection were performed to the barcode sequences associated with the probes. Detection was performed using intermediate probes that hybridized to the RCPs and detectably labeled detection oligonucleotides (DOs) that hybridized to the intermediate probes.

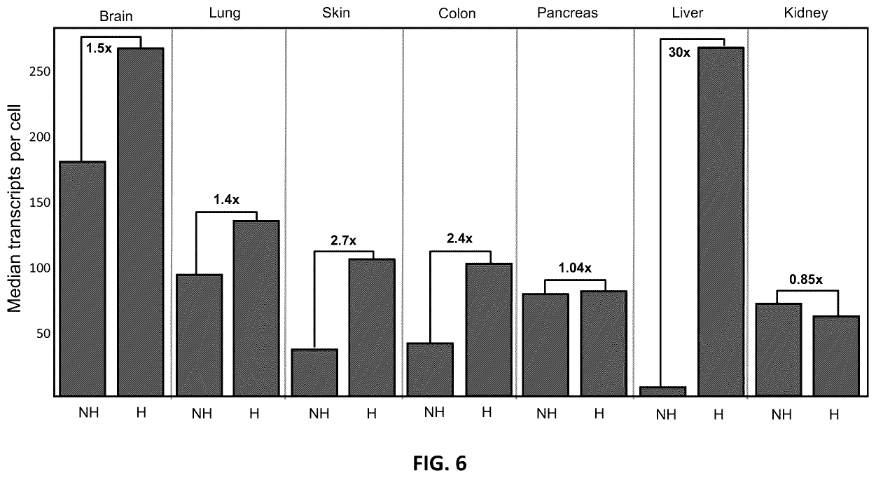

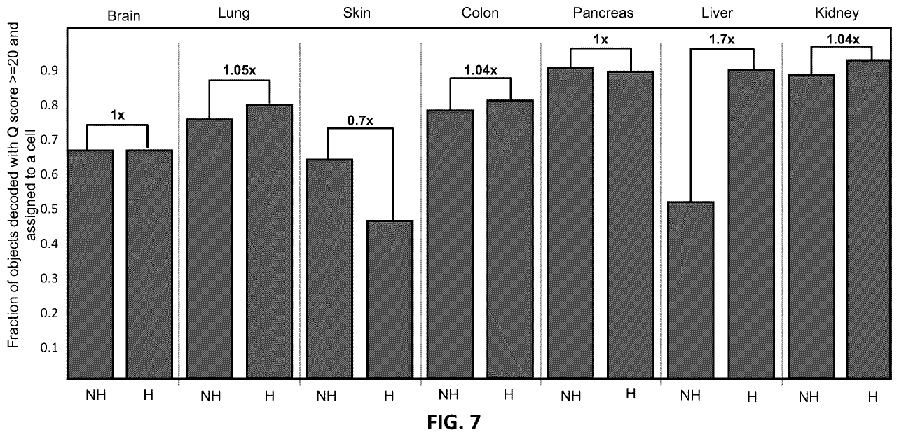

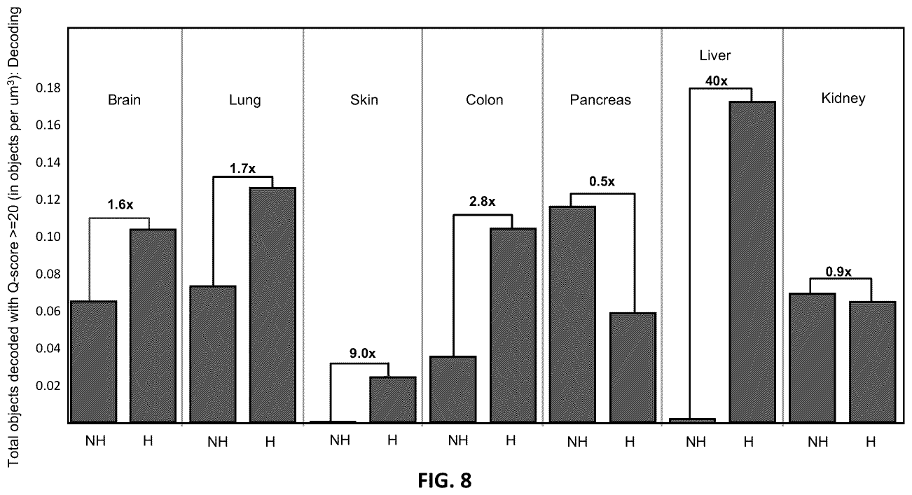

Results: RNA decoding results are shown in FIGS. 6-8. Tethering RNAs and embedding FFPE human TMA samples in hydrogel improved RNA transcript detection. Specifically, median RCPs per cell increased in hydrogel-embedded samples (FIG. 6), the fraction of barcoded, confidently mapped RCP reads with cell-associated barcodes increased in hydrogel-embedded samples (FIG. 7), and the total number of RCPs increased in hydrogel-embedded samples (FIG. 8).

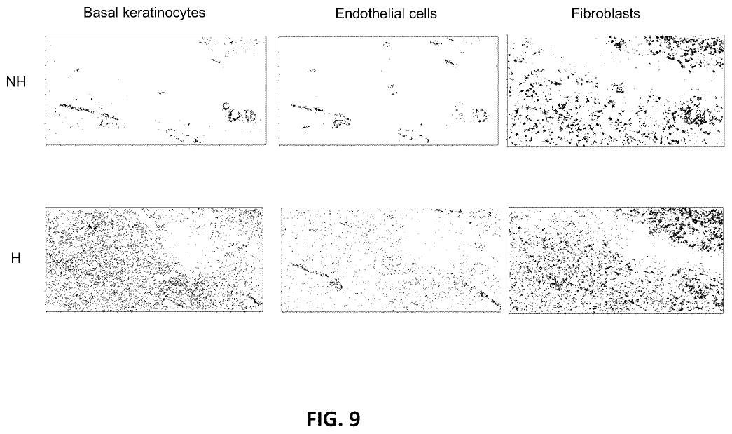

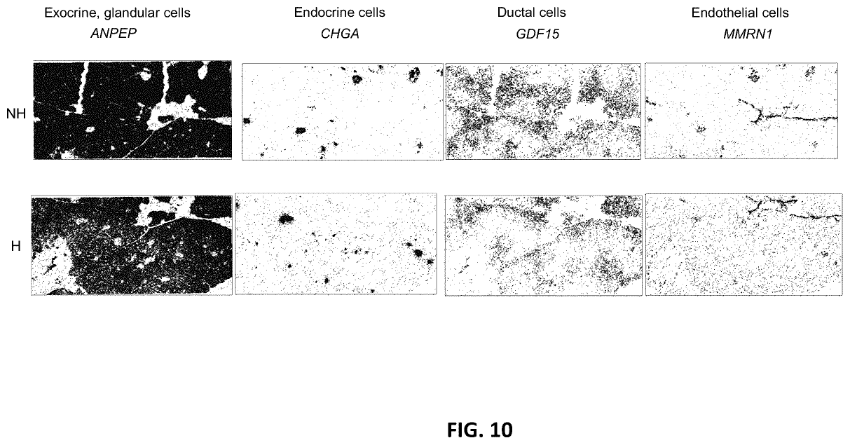

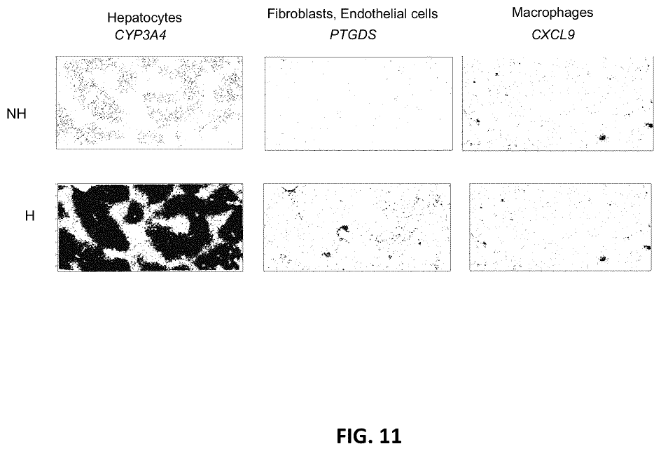

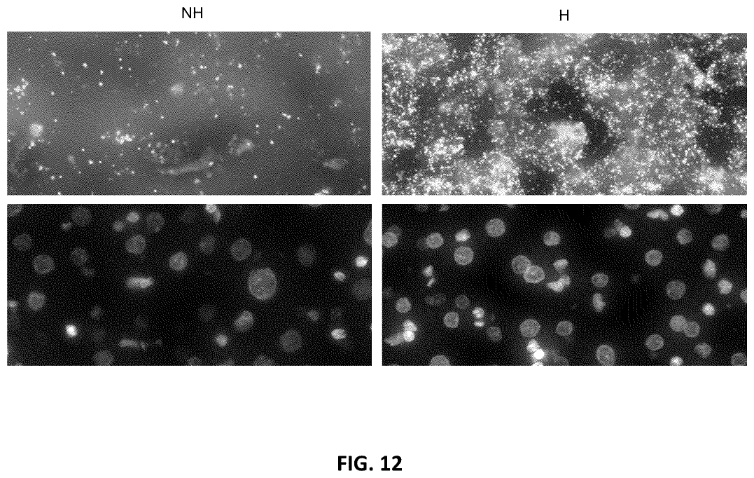

Clustering was performed to analyze gene localization in the various tissue samples. Visualization of RCPs in areas of the same size in skin (FIG. 9), pancreas (FIG. 10), and liver samples (FIGS. 11 and 12) demonstrated that across different cell types, fragmented RNA in FFPE human TMA samples can be retained during transcriptomic analysis by conversion of fragmented 3′ ends of RNA to functional groups and embedding the samples in hydrogel. These data demonstrate that standard processing of tissue samples may result in loss of RNA and that tethering RNA using the RNA immobilization methods described throughout the present disclosure can retain fragmented RNA in the sample for detection.

### Example 3: Treatment of Fragmented RNA in Mouse Brain (mBrain) Samples and Tissue

Clearing with Sodium Dodecyl Sulfate (SDS) and Proteinase K (PK).

This example describes immobilization of RNA contained in formalin-fixed paraffin-embedded (FFPE) mouse brain (mBrain) tissue samples and subsequent treatment for tissue clearing with SDS and PK for imaging and transcriptomic analysis.

Sample Preparation: Four separate FFPE mBrain samples were baked at 60° C. for 2 hr and deparaffinized via (a) xylene treatment-incubation in 100% xylene twice for (2×) 10 min each, followed by (b) serial ethanol treatment-incubation with 100% ethanol (2×) 3 min each, then 96% ethanol 2×3 min each, then 70% ethanol once (1×) 3 min, followed by brief immersion in nuclease-free water (1×20 sec). The FFPE mBrain samples were not de-crosslinked or permeabilized.

After sample preparation, the four separate FFPE mBrain samples were designated as follows: a non-embedded control sample that did not undergo RNA tethering treatment (“SOP”); a sample processed by the standard procedures described above and treated to tether RNA and embedded in a hydrogel as described in Example 2 (“Tethered”); a sample treated with 200 μg/ml Proteinase K and 1% SDS for 3 min at 50° C. in PBS 8.5 pH after NAIO₄ treatment and hydrogel embedding (“Tethered SDS/PK 3 min”); and a sample treated with 200 μg/ml Proteinase K and 1% SDS for 6 min at 50° C. in PBS 8.5 pH after NAIO₄ treatment and hydrogel embedding (“Tethered SDS/PK 6 min”).

The embedded samples were contacted with a panel of mouse brain genes using one circularizable probe per gene and ligation was performed by incubating with a ligase to form circularized probes. For RCA, the samples were washed and then incubated in an RCA reaction mixture (containing Phi29 reaction buffer, dNTPs, Phi29 polymerase) to generate RCA products (RCPs) using the circularized probes as template. Detection was performed to the barcode sequences associated with the probes, using intermediate probes that hybridized to the RCPs and detectably labeled detection oligonucleotides (DOs) that hybridized to the intermediate probes and imaged after one cycle.

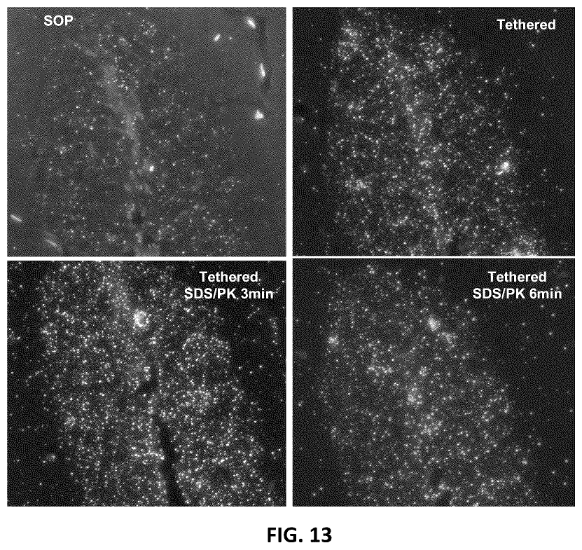

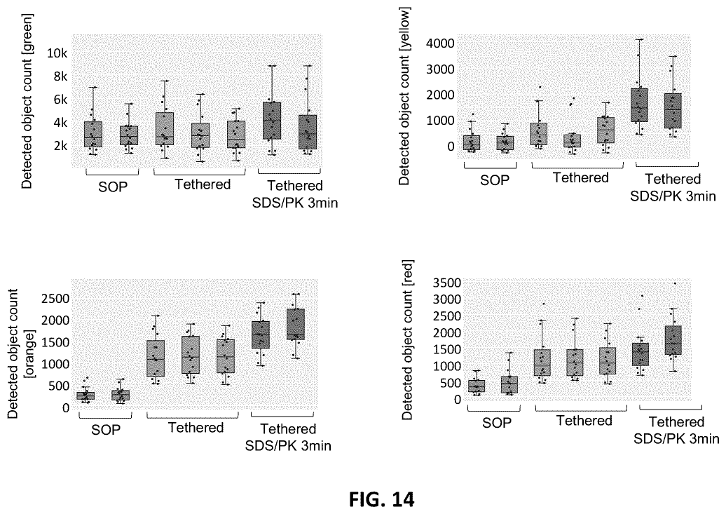

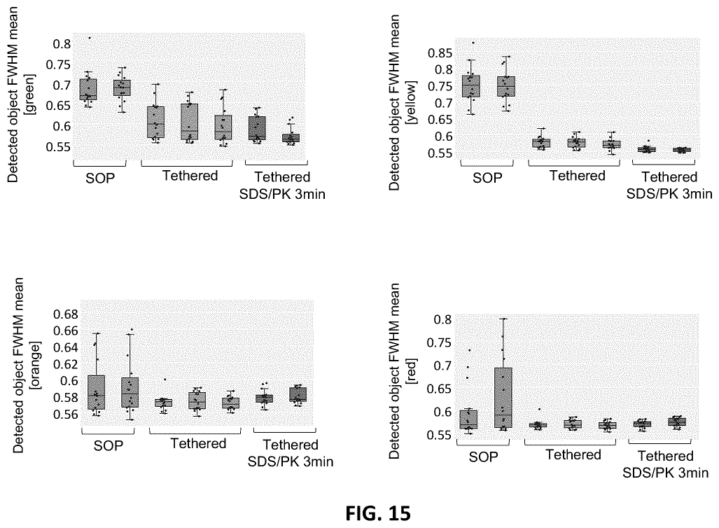

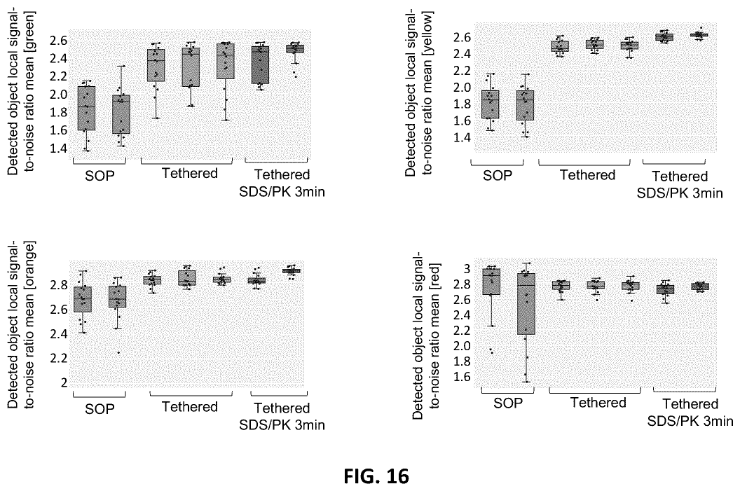

Results: Treating RNA-tethered and hydrogel embedded mBrain samples for 3 min with PK and SDS significantly improved RCP resolution by imaging (FIG. 13). Further, the total number of detected RCPs increased when the samples were treated with PK and SDS for 3 min (FIG. 14). Additionally, the size of RCPs in hydrogel embedded mBrain samples were significantly smaller than the RCPs detected in SOP samples (FIG. 15), and reductions in mean local signal-to-background ratio were observed (FIG. 16).

These data demonstrate that RCP detection and imaging is further improved when hydrogel embedded samples with tethered RNA were treated with SDS and PK. In addition, detection of 3′ tethered RNA as described herein may be performed using only 1 probe per gene as sensitivity can be increased with the tethering, clearing and embedding procedures.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

### ENUMERATED EMBODIMENTS

Embodiment 1. A method, comprising:
(a) contacting a biological sample comprising a ribonucleic acid with a formylation reagent, wherein the ribonucleic acid comprises a 2′,3′-vicinal diol and the formylation reagent converts the 2′,3′-vicinal diol moiety into a 2′3′-dialdehyde moiety; and
(b) contacting the biological sample with an attachment agent comprising a first reactive group capable of reacting with at least one aldehyde of the 2′,3′-dialdehyde moiety of the ribonucleic acid to form a covalent bond and an attachment moiety capable of attaching covalently or non-covalently to an exogenous or endogenous molecule in the biological sample, thereby immobilizing the ribonucleic acid in the biological sample.

Embodiment 2. The method of embodiment 1, wherein the ribonucleic acid is a fragmented ribonucleic acid.

Embodiment 3. The method of embodiment 1 or 2, wherein the 2′,3′-vicinal diol is a fragmented 3′ end of the ribonucleic acid.

Embodiment 4. The method of any one of embodiments 1 to 3, wherein the 2′,3′-vicinal diol is generated from a fragmented ribonucleic acid having a 2′,3′cyclo-phosphate fragmentation at the 3′-terminal end or a 2′ hydroxyl and a 3′ phosphate fragmentation at the 3′-terminal end.

Embodiment 5. The method of any one of embodiments 1 to 4, wherein the 2′,3′-vicinal diol is provided by contacting the fragmented ribonucleic acid with a 3′ phosphatase.

Embodiment 6. The method of embodiment 5, wherein the 3′ phosphatase is T4 polynucleotide kinase.

Embodiment 7. The method of any one of embodiments 1 to 6, wherein the biological sample is treated with degradation agent to induce fragmentation of ribonucleic acids, optionally wherein the degradation agent is an RNase or restriction enzyme.

Embodiment 8. The method of any of embodiments 1 to 7, wherein the ribonucleic acid comprises a 5′ cap and the 2′,3′-vicinal diol is in the 5′ cap.

Embodiment 9. The method of embodiment 8, wherein the 5′ cap is a 7-methylguanosine cap. Embodiment 10. The method of any one of embodiments 1 to 9, wherein the formylation reagent is an oxidant, optionally wherein the oxidant is sodium (meta)periodate.

Embodiment 11. The method of any one of embodiments 1 to 10, wherein the attachment moiety comprises a second reactive group capable of reacting with the exogenous or endogenous molecule in the biological sample to form a covalent bond.

Embodiment 12. The method of embodiment 11, wherein the attachment agent is 2-aminoethyl methacrylate.

Embodiment 13. The method of embodiment 12, wherein the step of contacting the biological sample and attachment agent further comprises contacting the biological sample with a reducing agent, optionally wherein the reducing agent is sodium borohydride.

Embodiment 14. The method of any one of embodiments 1 to 10, wherein the attachment moiety is capable of attaching non-covalently to the exogenous or endogenous molecule in the biological sample.

Embodiment 15. The method of embodiment 14, wherein the attachment agent is biotinylated.

Embodiment 16. The method of any of embodiments 1 to 15, wherein the exogenous molecule is a matrix-forming agent and the method further comprises:
(c) contacting the biological sample with a matrix-forming agent; and
(d) forming a three-dimensional polymerized matrix from the matrix-forming agent, thereby embedding the biological sample in the three-dimensional polymerized matrix and anchoring the ribonucleic acid to the three-dimensional polymerized matrix.

Embodiment 17. The method of embodiment 16, further comprising clearing the biological sample embedded in the three-dimensional polymerized matrix.

Embodiment 18. The method of embodiment 17, wherein the biological sample is cleared with a detergent, a lipase, and/or a protease.

Embodiment 19. The method of embodiment 18, wherein the detergent comprises a non-ionic surfactant or anionic surfactant, optionally wherein the detergent comprises SDS, tergitol, NP-40, saponin, polyethylene glycol p-(1,1,3,3-tetramethylbutyl)-phenyl ether, or polysorbate 20, or any combinations thereof.

Embodiment 20. The method of embodiment 18, wherein the protease comprises proteinase K, pepsin, or collagenase, trypsin, dispase, thermolysin, or alpha-chymotrypsin, or any combinations thereof, optionally wherein the protease comprises Liberase™.

Embodiment 21. The method of embodiment 18, wherein the lipase comprises a pancreatic, hepatic and/or lysosomal lipase, or any combinations thereof, optionally wherein the lipase comprises sphingomyelinase or esterase, or a combination thereof.

Embodiment 22. The method of any one of embodiments 17 to 21, further comprising contacting the biological sample with a probe or probe set that binds directly or indirectly to the ribonucleic acid, optionally wherein the probe or probe set is a detectable probe.

Embodiment 23. The method of embodiment 22, wherein the probe or probe set is a circular or circularizable probe or probe set, optionally wherein the method comprises circularizing the circularizable probe or probe set using the ribonucleic acid or a product thereof as a template, optionally wherein the method comprises generating an RCA product using the circular or circularizable probe as a template.

Embodiment 24. The method of embodiment 22 or 23, comprising imaging the biological sample to detect the probe or probe set or the RCA product.

Embodiment 25. The method of embodiment 24, wherein imaging comprises detecting a signal associated with the probe or probe set or the RCA product, optionally wherein the signal is from a fluorescently labeled probe that directly or indirectly binds to the probe or probe set or the RCA product.

Embodiment 26. The method of any of embodiments 22-25, wherein the probe or probe set comprises a barcode sequence.

Embodiment 27. The method of embodiment 26, wherein the method comprises detecting the barcode sequence or a complement thereof in the probe or probe set or in a product of the probe or probe set.

Embodiment 28. A method of analyzing a fragmented ribonucleic acid in a biological sample, the method comprising:
(a) contacting the biological sample comprising a fragmented ribonucleic acid with a 3′ phosphatase to provide a fragmented ribonucleic acid comprising a 2′,3′-vicinal diol;
(b) contacting the biological sample with formylation reagent, wherein the formylation reagent converts the 2′,3′-vicinal diol moiety into 2′3′-dialdehyde moiety;
(c) contacting the biological sample with an attachment agent comprising a first reactive group capable of reacting with at least one aldehyde of the 2′,3′-dialdehyde moiety of the fragmented ribonucleic acid to form a covalent bond and a second reactive group capable of reacting with a matrix-forming agent to form a covalent bond;
(d) contacting the biological sample with a matrix-forming agent;
(e) forming a three-dimensional polymerized matrix from the matrix-forming agent, thereby embedding the biological sample in the three-dimensional polymerized matrix and anchoring the fragmented ribonucleic acid to the three-dimensional polymerized matrix;
(f) clearing the biological sample;
(g) contacting the biological sample with a probe or probe set that binds directly or indirectly to the ribonucleic acid; and
(h) detecting the probe or a product thereof at a location in the matrix.

Embodiment 29. The method of embodiment 28, wherein the fragmented ribonucleic acid in step (a) has a 2′,3′cyclo-phosphate fragmentation at the 3′-terminal end or a 2′ hydroxyl and a 3′ phosphate fragmentation at the 3′-terminal end, and wherein the 3′ phosphatase catalyzes the formation of the 2′,3′-vicinal diol.

Embodiment 30. The method of embodiment 28 or embodiment 29, wherein the 3′ phosphatase is T4 polynucleotide kinase.

Embodiment 31. The method of any one of embodiments 28 to 30, wherein the fragmented ribonucleic acid of step (a) is generated by treating the biological sample with a degradation agent to induce fragmentation of ribonucleic acids, optionally wherein the degradation agent is an RNase or restriction enzyme.

Embodiment 32. The method of any one of embodiments 28 to 31, wherein the fragmented ribonucleic acid further comprises an additional vicinal diol moiety provided by a 5′ cap, optionally wherein the 5′ cap is a 7-methylguanosine cap.

Embodiment 33. The method of any one of embodiments 28 to 32, wherein the formylation reagent is sodium (meta)periodate.

Embodiment 34. The method of any one of embodiments 28 to 33, wherein the attachment agent is 2-aminoethyl methacrylate.

Embodiment 35. The method of embodiment 34, wherein the step of contacting the biological sample and attachment agent further comprises contacting the biological sample with a reducing agent, optionally wherein the reducing agent is sodium borohydride.

Embodiment 36. The method of any one of embodiments 28 to 35, wherein the biological sample is cleared with a detergent, a lipase, and/or a protease.

Embodiment 37. The method of embodiment 36, wherein the detergent comprises a non-ionic surfactant or anionic surfactant, optionally wherein the detergent comprises SDS, tergitol, NP-40, saponin, polyethylene glycol p-(1,1,3,3-tetramethylbutyl)-phenyl ether, or polysorbate 20, or any combinations thereof.

Embodiment 38. The method of embodiment 36, wherein the protease comprises proteinase K, pepsin, or collagenase, trypsin, dispase, thermolysin, or alpha-chymotrypsin, or any combinations thereof, optionally wherein the protease comprises Liberase™.

Embodiment 39. The method of embodiment 36, wherein the lipase comprises a pancreatic, hepatic and/or lysosomal lipase, or any combinations thereof, optionally wherein the lipase comprises sphingomyelinase or esterase, or a combination thereof.

Embodiment 40. The method of any one of embodiments 28 to 39, wherein the probe or probe set is a circular or circularizable probe or probe set capable of binding the fragmented ribonucleic acid.

Embodiment 41. The method of embodiment 40, further comprising: generating a rolling circle amplification (RCA) product of the circular or circularizable probe or probe set; and detecting a signal associated with a fluorescently labeled probe that directly or indirectly binds to the rolling circle amplification product.

Embodiment 42. A method of analyzing a biological sample, the method comprising:
(a) contacting the biological sample comprising fragmented ribonucleic acid with T4 polynucleotide kinase, wherein the T4 polynucleotide kinase catalyzes formation of a 2′,3′-vicinal diol moiety on the fragmented ribonucleic acid;
(b) contacting the biological sample with sodium (meta)periodate, and wherein the sodium (meta)periodate converts the 2′,3′-vicinal diol to a 2′3′-dialdehyde moiety;
(c) contacting the biological sample with 2-aminoethyl methacrylate and sodium borohydride,
wherein the 2-aminoethyl methacrylate reacts with at least one aldehyde of the 2′3′-dialdehyde moiety of the ribonucleic acid to form 3′-aminoethylene-methacrylate;
(d) contacting the biological sample with a matrix-forming agent;
(e) forming a polymerized matrix from the matrix-forming agent, thereby embedding the biological sample in the three-dimensional polymerized matrix and anchoring the fragmented ribonucleic acid to the three-dimensional polymerized matrix;
(f) clearing the biological sample;
(g) contacting the biological sample with a circular or circularizable probe or probe set, wherein the circular or circularizable probe binds the ribonucleic acid;
(h) generating a rolling circle amplification (RCA) product of the circular or circularizable probe or probe set; and
(i) detecting a signal associated with a fluorescently labeled probe that directly or indirectly binds to the rolling circle amplification product.

Embodiment 43. The method of any one of embodiments 1 to 42, wherein the biological sample comprises cells or cellular components.

Embodiment 44. The method of any one of embodiments 1 to 43, wherein the biological sample is a tissue sample.

Embodiment 45. The method of embodiment 44, wherein the tissue sample is a tissue slice between about 1 μm and about 50 μm in thickness.

Embodiment 46. The method of any one of embodiments 1 to 45, wherein the biological sample is fixed.

Embodiment 47. The method of embodiment 46, wherein the biological sample is a formalin-fixed, paraffin-embedded (FFPE) sample, a fresh tissue sample, or a frozen tissue sample.

Embodiment 48. The method of any one of embodiments 1 to 47, further comprising staining, permeabilizing, cross-linking, expanding, and/or de-cross-linking the biological sample embedded in the three-dimensional polymerized matrix.

Embodiment 49. The method of any one of embodiments 1 to 48, wherein the biological sample and fragmented ribonucleic acid are treated with a ribonuclease inhibitor.

Embodiment 50. The method of any one of embodiments 1 to 49, wherein the step of contacting the biological sample with the 3′ phosphatase is not performed in the presence of ammonium ions, phosphate ions, or metal chelators.

Embodiment 51. The method of any one of embodiments 1 to 50, wherein the step of contacting the biological sample with the 3′ phosphatase is not performed in the presence of sodium chloride or potassium chloride buffer having a concentration of greater than 50 mM.

Embodiment 52. The method of any one of embodiments 1 to 51, wherein the matrix-forming agent comprises polyacrylamide, cellulose, alginate, polyamide, cross-linked agarose, cross-linked dextran or cross-linked polyethylene glycol.

Embodiment 53. The method of any one of embodiments 1 to 52, wherein the three-dimensional polymerized matrix is formed by subjecting the matrix-forming agent to polymerization.

Embodiment 54. The method of embodiment 53, wherein the polymerization is initiated by adding a polymerization-inducing catalyst, UV light or functional cross-linkers.

Embodiment 55. The method of any one of embodiments 1 to 54, wherein the fragmented ribonucleic acid is fragmented mRNA.

Embodiment 56. A kit for analyzing fragmented nucleic acids in a biological sample, comprising:
(a) a 3′ phosphatase, optionally wherein the 3′ phosphatase is T4 polynucleotide kinase;
(b) a formylation reagent, optionally wherein the reagent is an oxidant, optionally wherein the oxidant is sodium metaperiodate;
(c) an attachment agent comprising a reactive group capable of reacting with an aldehyde moiety and an attachment moiety capable of attaching covalently or non-covalently to an exogenous or endogenous molecule in the biological sample, optionally wherein the kit further comprises one or more reagents for reacting the attachment agent with the aldehyde;
(d) an exogenous molecule capable of attaching covalently or non-covalently to the attachment moiety, optionally wherein the exogenous molecule is a matrix-forming agent for embedding the biological sample in a three-dimensional polymerized matrix;
(e) a clearing agent, optionally wherein the clearing agent is a detergent, a lipase, and/or a protease; and
(f) instructions for use.

1. A method, comprising:
contacting a biological sample comprising a ribonucleic acid with a formylation reagent, wherein the ribonucleic acid comprises a 2′,3′-vicinal diol and the formylation reagent converts the 2′,3′-vicinal diol moiety into a 2′3′-dialdehyde moiety; and
contacting the biological sample with an attachment agent comprising at least one aldehyde-reactive group capable of reacting with at least one aldehyde of the 2′,3′-dialdehyde moiety of the ribonucleic acid to form a covalent bond and at least one attachment moiety capable of attaching covalently or non-covalently to an exogenous or endogenous molecule in the biological sample,
thereby immobilizing the ribonucleic acid in the biological sample.

2. (canceled)

3. (canceled)

4. The method of claim 1, wherein the 2′,3′-vicinal diol is generated from a fragmented ribonucleic acid having a 2′,3′cyclo-phosphate fragmentation at the 3′-terminal end or a 2′ hydroxyl and a 3′ phosphate fragmentation at the 3′-terminal end.

5. The method of claim 1, wherein the 2′,3′-vicinal diol is provided by contacting the fragmented ribonucleic acid with a 3′ phosphatase.

6. The method of claim 5, wherein the 3′ phosphatase is T4 polynucleotide kinase.

7. (canceled)

8. The method of claim 1, wherein the ribonucleic acid comprises a 5′ cap and the 2′,3′-vicinal diol is in the 5′ cap.

9. (canceled)

10. The method of claim 1, wherein the formylation reagent is sodium (meta)periodate.

11. The method of claim 1, wherein the attachment moiety is capable of attaching covalently to an exogenous or endogenous molecule in the biological sample.

12-16. (canceled)

17. The method of claim 1, wherein the attachment agent is a compound of formula (I) 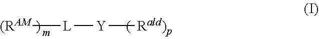
or a salt thereof,
wherein each R^{ald} is independently an aldehyde-reactive group capable of reacting with at least one aldehyde of the 2′,3′-dialdehyde moiety of the ribonucleic acid to form a covalent bond;
Y is —CH₂CH₂— or —O—;
L is a bond or a linker moiety;
each R^{AM} is independently an attachment moiety capable of attaching covalently or non-covalently to an exogenous or endogenous molecule in the biological sample;
m is an integer from 1 to 4; and
p is an integer from 1 to 4.

18-23. (canceled)

24. The method of claim 1, wherein the attachment agent is a compound of formula (I-a) 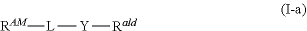
or a salt thereof,
wherein R^{ald} is an aldehyde-reactive group;
Y is —CH₂CH₂— or —O—;
L is a bond or a linker moiety; and
R^{AM} is an attachment moiety.

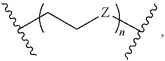

25. The method of claim 24, wherein L is wherein
Z is CH₂, O, S, or NH; and
n is an integer from 0 to 50.

26. The method of claim 24 wherein the compound of formula (I-a) is a compound of formula (III-a) 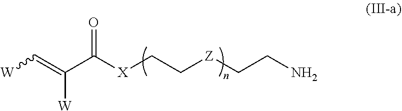
or a salt thereof, wherein
each W is independently H or CH₃;
X is NH or 0;
Z is CH₂, O, S, or NH; and
n is an integer from 0 to 50.

27. The method of claim 24, wherein the attachment agent is N-(2-aminoethyl)methacrylamide, 2-aminoethyl methacrylate, or 2-aminoethyl (E)-but-2-enoate.

28. The method of claim 27, wherein the attachment agent is N-(2-aminoethyl)methacrylamide.

29. (canceled)

30. (canceled)

31. The method of claim 24, wherein the compound of formula (I-a) is a compound of formula (III-b) 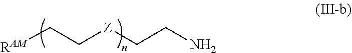
or a salt thereof, wherein
R^{AM} is a biotinyl moiety, a dextrin moiety, a click moiety, a thiol moiety, a norbornenyl moiety, a furanyl moiety, an alkyl ester moiety, or a maleimidyl moiety;
Z is CH₂, O, S, or NH; and
n is an integer from 0 to 50.

32. The method of claim 24, wherein the step of contacting the biological sample and attachment agent further comprises contacting the biological sample with a reducing agent, optionally wherein the reducing agent is sodium borohydride.

33. The method of claim 24, wherein the exogenous molecule is a matrix-forming agent and the method further comprises:
contacting the biological sample with a matrix-forming agent; and
forming a three-dimensional polymerized matrix from the matrix-forming agent, thereby embedding the biological sample in the three-dimensional polymerized matrix and anchoring the ribonucleic acid to the three-dimensional polymerized matrix.

34. The method of claim 33, further comprising clearing the biological sample embedded in the three-dimensional polymerized matrix.

35-44. (canceled)

45. The method of claim 24, further comprising contacting the biological sample with a probe or probe set that binds directly or indirectly to the ribonucleic acid, optionally wherein the probe or probe set is a detectable probe.

46. The method of claim 24, wherein the probe or probe set is a circular or circularizable probe or probe set, optionally wherein the method comprises circularizing the circularizable probe or probe set using the ribonucleic acid or a product thereof as a template, optionally wherein the method comprises generating an RCA product using the circular or circularizable probe as a template.

47-67. (canceled)

68. A method of analyzing a biological sample, the method comprising:
contacting the biological sample comprising fragmented ribonucleic acid with T4 polynucleotide kinase, wherein the T4 polynucleotide kinase catalyzes formation of a 2′,3′-vicinal diol moiety on the fragmented ribonucleic acid;
contacting the biological sample with sodium (meta)periodate, and wherein the sodium (meta)periodate converts the 2′,3′-vicinal diol to a 2′3′-dialdehyde moiety;
contacting the biological sample with sodium borohydride and an attachment agent selected from N-(2-aminoethyl)methacrylamide, 2-aminoethyl methacrylate, and 2-aminoethyl (E)-but-2-enoate, wherein the N-(2-aminoethyl)methacrylamide, 2-aminoethyl methacrylate, or 2-aminoethyl (E)-but-2-enoate reacts with at least one aldehyde of the 2′3′-dialdehyde moiety of the ribonucleic acid to form 3′-aminoethylene-methacrylamide, 3′-aminoethylene-methacrylate, or 3′-aminoethyl (E)-but-2-enoate;
contacting the biological sample with a matrix-forming agent;
forming a polymerized matrix from the matrix-forming agent, thereby embedding the biological sample in the three-dimensional polymerized matrix and anchoring the fragmented ribonucleic acid to the three-dimensional polymerized matrix;
clearing the biological sample;
contacting the biological sample with a circular or circularizable probe or probe set, wherein the circular or circularizable probe binds the ribonucleic acid;
generating a rolling circle amplification (RCA) product of the circular or circularizable probe or probe set; and
detecting a signal associated with a fluorescently labeled probe that directly or indirectly binds to the rolling circle amplification product.

69-85. (canceled)
